(19) United States
(12) Patent Application Publication
(10) Pub. No.: US 2024/0131029 A1
(43) Pub. Date: Apr. 25, 2024

(54) SELECTIVE ROCK2 INHIBITION FOR TREATMENT OF EDEMA AND ASSOCIATED CONDITIONS

(71) Applicants: President and Fellows of Harvard College, Cambridge, MA (US); Trustees of Boston University, Boston, MA (US)

(72) Inventors: Esak Lee, Cambridge, MA (US); Christopher S. Chen, Boston, MA (US)

(73) Assignees: President and Fellows of Harvard College, Cambridge, MA (US); Trustees of Boston University, Boston, MA (US)

(21) Appl. No.: 18/275,731

(22) PCT Filed: Feb. 3, 2022
(86) PCT No.: PCT/US2022/015072

§ 371 Date: Aug. 3, 2023

Related U.S. Application Data
(60) Provisional application No. 63/228,404, filed on Aug. 2, 2021.
(60) Provisional application No. 63/209,324, filed on Jun. 10, 2021.
(60) Provisional application No. 63/145,526, filed on Feb. 4, 2021.

(51) Int. Cl. A61K 31/517; A61P 7/10

(52) U.S. Cl. CPC A61K 31/517; A61P 7/10

(57) ABSTRACT

The present disclosure provides methods and compositions for treating a condition associated with impaired lymphatic drainage.

### RELATED APPLICATIONS

This application claims the benefit under 35 U.S.C. § 119(e) of U.S. provisional application No. 63/145,526, filed Feb. 4, 2021, U.S. provisional application No. 63/209,324, filed Jun. 10, 2021, and U.S. provisional application No. 63/228,404, filed Aug. 2, 2021, each of which is incorporated by reference herein in its entirety.

### FEDERALLY SPONSORED RESEARCH

This invention was made with government support under EB025765, EB008396, EB000262, and HL129733 awarded by National Institutes of Health. The government has certain rights in the invention.

### BACKGROUND

Conditions associated with impaired lymphatic drainage, such as lymphedema, are major morbidities with obscure mechanisms. Lymphedema, the excess accumulation of interstitial fluid resulting from impaired fluid drainage into the lymphatic vasculature (1, 2), affects more than 150 million individuals worldwide, and yet, with no effective treatments, the standard of care is largely palliative (3). A lack of experimental models of normal and impaired lymphatic drainage has been a major obstacle to better understanding and treatment of lymphedema.

### SUMMARY

Provided herein, in some aspects, are methods and compositions for treating a subject having or at risk of a condition associated with impaired lymphatic drainage (e.g., lymphedema) using a selective Rho-associated kinase 2 (ROCK2) inhibitor. Also provided herein is a biomimetic model of lymphatic vessels, which was used to identify a ROCK mechanism in regulating lymphatic drainage. This microfluidic culture model of lymphatic vessels draining interstitial fluid was engineered and used to demonstrate that inflammatory cytokines that disrupt blood vessel junctions instead tightened lymphatic cell-cell junctions and impeded lymphatic drainage. The data herein shows that inhibition of ROCK normalized fluid drainage under cytokine challenge by loosening lymphatic junctions and tightening blood vessel junctions in culture, and completely reversed lymphedema in a murine model. Further a ROCK2/JAM-A/ZO-1 complex was identified, which relates to the distinct cytokine response of lymphatic junctions.

Thus, some aspects of the present disclosure provide a method of administering a selective ROCK2 inhibitor to a subject (e.g., a human subject) having or at risk of a condition associated with impaired lymphatic drainage. In some embodiments, the subject has a condition associated with impaired lymphatic drainage. In some embodiments, the condition is edema. In some embodiments, the condition is lymphedema. The lymphedema may be primary lymphedema or secondary lymphedema.

Some aspects of the present disclosure provide a method comprising administering to a subject a selective ROCK2 inhibitor in an amount effective to rescue vascular barrier function. In some embodiments, the amount of the selective ROCK2 inhibitor is effective in reducing fluid leakage from blood vessels in the subject, relative to a control. In some embodiments, the amount of the selective ROCK2 inhibitor is effective in increasing lymphatic drainage in the subject, relative to a control.

Other aspects of the present disclosure provide a method comprising administering to a subject a selective ROCK2 inhibitor in an amount effective for improving lymphatic drainage at a lymphatic junction in the subject, relative to a control.

Yet aspects of the present disclosure provide a method comprising administering to a subject a selective ROCK2 inhibitor in an amount effective in an amount effective for preventing formation of tight lymphatic junctions in the subject.

In some embodiments, the selective ROCK2 inhibitor is administered in an amount effective for alleviating a symptom associated with impaired lymphatic drainage. In some embodiments, the symptom is swelling in a body part of the subject.

In some embodiments, the selective ROCK2 inhibitor is administered orally. In some embodiments, the selective ROCK2 inhibitor is formulated as a tablet. In some embodiments, the selective ROCK2 inhibitor is administered as a dose of 200-400 mg.

Other aspects of the present disclosure provide a method comprising contacting dermal lymphatic endothelial cells with a selective ROCK2 inhibitor in an amount effective for preventing formation of tight lymphatic junctions.

Still other aspects of the present disclosure provide a method of contacting a tight lymphatic junction with a selective ROCK2 inhibitor in an amount effective for improving lymphatic drainage at the lymphatic junction, relative to a control.

In some embodiments, the selective ROCK2 inhibitor binds to ROCK2 and inhibits ROCK2 serine/threonine kinase activity.

In some embodiments, the selective ROCK2 inhibitor is selected from polypeptide inhibitors, polynucleotide inhibitors, and small molecule inhibitors. In some embodiments, the selective ROCK2 inhibitor is a small molecule inhibitor.

In some embodiments, the small molecule inhibitor binds ROCK2 with an IC₅₀ value of 50 nm-150 nM. In some embodiments, the small molecule inhibitor binds ROCK2 with an IC₅₀ value of 50 nm-150 nM, without binding PKA, PKG, PKC, or MRCK. In some embodiments, the small molecule inhibitor binds ROCK1 with an IC₅₀ value of 20,000-25,0000 nM. Other ROCK2 inhibitors may be used, including, in some embodiments, SR-3677 (TOCHRIS®) and Chroman 1 (MCE®).

In some embodiments, the selective ROCK2 small molecule inhibitor is 2-[3-[4-(1H-indazol-5-ylamino)-2-quinazolinyl]phenoxy]-N-(1-methylethyl)-acetamide (KD025).

In some embodiments, the selective ROCK2 small molecule inhibitor is selected from Compounds 1-559, or a pharmaceutically acceptable salt, stereoisomer, tautomer, isotopically labeled derivative, solvate, hydrate, polymorph, co-crystal, or prodrug thereof.

In some embodiments, the selective ROCK2 small molecule inhibitor is selected from a Compound of Table 1, or a pharmaceutically acceptable salt, stereoisomer, tautomer, isotopically labeled derivative, solvate, hydrate, polymorph, co-crystal, or prodrug thereof.

In some embodiments, the selective ROCK2 small molecule inhibitor is selected from a Compound of Table 2, or a pharmaceutically acceptable salt, stereoisomer, tautomer, isotopically labeled derivative, solvate, hydrate, polymorph, co-crystal, or prodrug thereof.

In some embodiments, the selective ROCK2 small molecule inhibitor is selected from a Compound of Table 3, or a pharmaceutically acceptable salt, stereoisomer, tautomer, isotopically labeled derivative, solvate, hydrate, polymorph, co-crystal, or prodrug thereof.

In some embodiments, the selective ROCK2 small molecule inhibitor is selected from a Compound of Table 4, or a pharmaceutically acceptable salt, stereoisomer, tautomer, isotopically labeled derivative, solvate, hydrate, polymorph, co-crystal, or prodrug thereof.

Yet other aspects of the present disclosure provide a biomimetic model of lymphatic vessels, comprising a polymer-based housing that comprises at least two parallel cylindrical channels within a three-dimensional gel, wherein human dermal cells are seeded in one of the cylindrical channels, optionally wherein the model further comprises a lymphangiogenic growth factor.

Still other aspects of the present disclosure provide methods, comprising providing a biomimetic device that comprises a polymer-based housing that comprises at least two parallel cylindrical channels within a three-dimensional gel, wherein human dermal cells are seeded in one of the channels to generate an engineered lymphatic vessel that expresses lymphatic markers, and stimulating the human dermal cells with a lymphangiogenic growth factor to trigger lymphatic sprouting.

In some embodiments, the methods further comprise pressurizing the other of the channels with a surrogate lymph fluid composed of fluorescently labeled lymph molecules, including fatty acids, phospholipids, albumin, and insoluble particles, and assessing lymph fluid drained by the engineered lymphatic vessel.

In some embodiments, the polymer-based housing is selected from polydimethylsiloxane (PDMS), polypropylene, polystyrene, polyurethanes, polycaprolactone, polymethylmethacrylate, and polyacetate.

In some embodiments, the gel is selected from collagen gel, fibrin, materiel, synthetic hydrogel, and hydrogel made from other extracellular matrix (ECM) components.

In some embodiments, the human dermal cells are human dermal lymphatic endothelial cells (LECs).

In some embodiments, the LECs exhibit appropriate apical-basal orientation.

In some embodiments, the lymphangiogenic growth factor is vascular endothelial growth factor C (VEGFC).

In some embodiments, the engineered lymphatic vessel expresses lymphatic endothelial markers.

### BRIEF DESCRIPTION OF THE DRAWINGS

FIGS. 1A-1J. Lymphatics-on-chip recapitulates lymphatic structure, drainage, and dysfunction. (FIG. 1A) A schematic of the lymphatics-on-chip. (FIG. 1B) Apical Podoplanin (PDPN) expression on the luminal side of the vessel. (FIG. 1C) Lymphatic sprouting in response to VEGFC stimulation. (FIG. 1D) Immunostaining of LEC-generated lymphatic vessels and BEC-generated blood vessels with tight junction markers (JAM-A, ZO-1). (FIG. 1E) Adherens junction staining. Red stars represent portal-like structures. (FIG. 1F) F-actin staining confirming the portal-like structure in lymphatics. (FIG. 1G) Percent drainage of lymph molecules through LEC and BEC-line channels as compared to acellular channels (No cell). (FIG. 1H) Representative images of BODIPY-C16 fatty acid drainage by LEC-generated lymphatic vessels and BEC-generated blood vessels. (FIG. 1I) Percent lymphatic drainage after cytokine exposure. (FIG. 1J) VE-cadherin staining of engineered lymphatics after cytokine exposure. Scale bars (FIGS. 3A, 3B, 3C, 3D, 3E, 3H)=100 μm; scale bars (FIGS. 3F, 3J)=50 μm; * (p<0.05) and ** (p<0.01) indicate statistical significance. FIGS. 2A-2J. ROCK inhibition normalizes LEC and BEC junctions, and reverses lymphedema in vivo. (FIG. 2A) Percent lymphatic drainage with Y27632. (FIG. 2B) VE-cadherin staining of LEC exposed to Y27632. (FIG. 2C) Percent lymphatic drainage with Y27632, Fasudil, and Ripasudil. (FIG. 2D) Dye-leakage based permeability assays and VE-cadherin images of engineered blood vessels. (FIG. 2E) Permeability coefficient of the engineered blood vessels in inflammation with or without Y27632. (FIG. 2F) Effects of ROCK inhibition on LEC and BEC junctions. (FIG. 2G) Tail volume change with Y27632 in lymphedema model. (FIG. 2H) Drainage to the iliac lymph nodes 1 h after intradermal injection of Alexa647-conjugated albumin into tails of mice treated with Y27632 or vehicle. (FIG. 2I) Tail volume change with delayed Y27632 exposure. (FIG. 2J) Representative images of tails before and after Y27632 treatment. Scale bars (FIG. 2B and FIG. 2D, lower panel)=50 μm; scale bars (FIG. 2D, upper panel)=200 μm; scale bars (J)=5 mm; * (p<0.05), ** (p<0.01), and *** (p<0.001) indicate statistical significance.

FIGS. 3A-3L. ROCK2 forms a unique tight junction complex in inflamed LECs, but not in BECs. (FIG. 3A) Western blot showing basal levels of ROCK1/2 expression in LECs and BECs. (FIG. 3B) Western blot showing ROCK1 downregulation in inflamed LECs. (FIG. 3C) siRNA mediated knock-down of ROCK1 and ROCK2 in LECs. (FIG. 3D) Percent lymphatic drainage by LEC knocked-down ROCK1 or ROCK2 in normal or IL-2 condition. (FIG. 3E) VE-cadherin images of the engineered LVs with or without ROCK1/2 knock-down in normal or IL-2 condition. (FIG. 3F) Permeability coefficient of the engineered BVs with or without ROCK1 knock-down in normal or IL-2 condition. (FIG. 3G) VE cadherin images of the engineered BVs with or without ROCK1 knock-down in normal or IL-2 condition. (FIG. 3H) Immunoprecipitation data showing ROCK2 interactions with junctional adhesion molecule-A (JAM-A) and zonula occludens-1 (ZO-1) in inflamed LECs or in ROCK1 kd LECs. (FIG. 3I) Immunofluorescent data showing ROCK2 localization to the junctional areas and enhanced tight junctions (JAM-A) in inflamed LECs or in ROCK1 kd LECs. (FIG. 3J) VE-cadherin images of the engineered LVs with JAM-A knock-down in normal or IL-2 condition. (FIG. 3K) Immunoprecipitation data showing that ROCK2-downregulating BEC does not form ROCK2-JAM-A complex in normal or IL-2 condition. (FIG. 3L) Percent lymphatic drainage with KD025 (20 μM), a ROCK2-specific inhibitor, in normal or IL-2 conditions. Scale bars 50 μm. *(p<0.05) indicates statistical significance.

FIG. 4A is a photograph showing that tail vein edema following cautery injury is visible by swelling of the tail (left) that is reduced by treatment with KD025 (right). FIG. 4B is a graph of quantified tail volume changes over time. FIG. 4C is a graph of data showing drainage to the iliac lymph nodes 1 hour after intradermal injection of Alexa647-conjugated albumin into tails of mice treated with KD025 or vehicle.

### DETAILED DESCRIPTION

Provided herein, in some aspects, are methods of administering a selective Rho-associated kinase 2 (ROCK2) inhibitor to a subject having or at risk of a condition associated with impaired lymphatic drainage. Also provided herein are methods of treating a condition associated with impaired lymphatic drainage. It should be understood that “treating a condition” in a subject includes administering to the subject an agent that alleviates a (one or more) symptom associated with the condition. For example, treating lymphedema in a limb (e.g., leg or arm) of a subject may include administering a selective ROCK2 inhibitor to the subject, and reducing or eliminating swelling of the limb.

Other aspects of the present disclosure provide methods that comprise contacting dermal lymphatic endothelial cells with a selective Rho-associated kinase 2 (ROCK2) inhibitor in an amount effective for preventing formation of tight lymphatic junctions. FIG. 2G, for example, shows the preventive effect of ROCK2 inhibition in lymphedema. Formation of tight junctions can be analyzed, in some embodiments, by staining VE-cadherin or JAM-A (junction markers) with LYVE-1 or Podoplanin (lymphatic vessel markers).

Still other aspects of the present disclosure provide methods that comprise contacting a tight lymphatic junction with a selective ROCK2 inhibitor in an amount effective for improving lymphatic drainage at the lymphatic junction, relative to a control.

### The Lymphatic System

The lymphatic system is an essential part of the immune system. It helps maintain tissue homeostasis, including interstitial protein transport, tissue fluid balance, and development of cellular immunity. Lymph nodes, which are located throughout the lymphatic system, contain large numbers of lymphocytes, macrophages and antigen presenting cells that together initiate the primary immune response. Specialized lymphatic endothelial cells (LECs) assist in the primary immune response by recruiting intravascular lymphocytes as they circulate. In addition, LECs are responsible for facilitating the transmigration of intravascular lymphocytes into the reticular meshwork where the lymphocytes can interact with antigen presenting cells. LECs have also been shown to be associated with chronic inflammation and cancers.

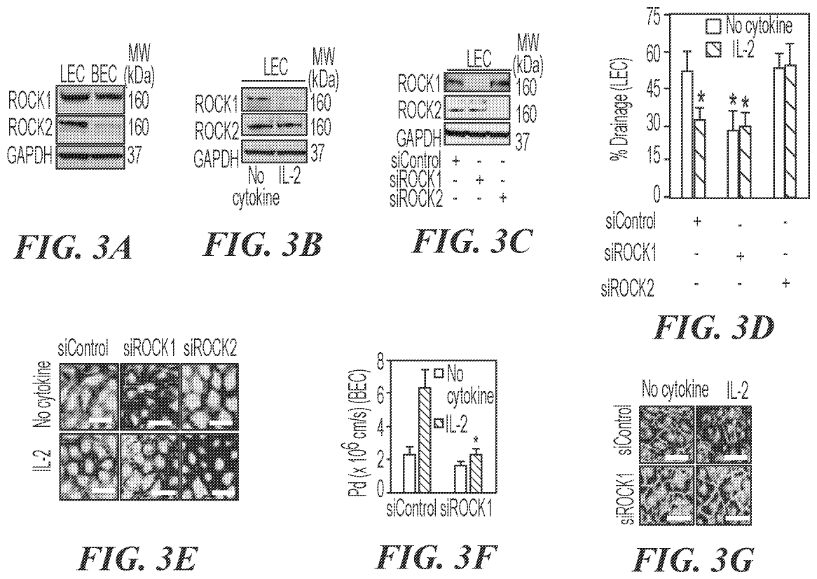

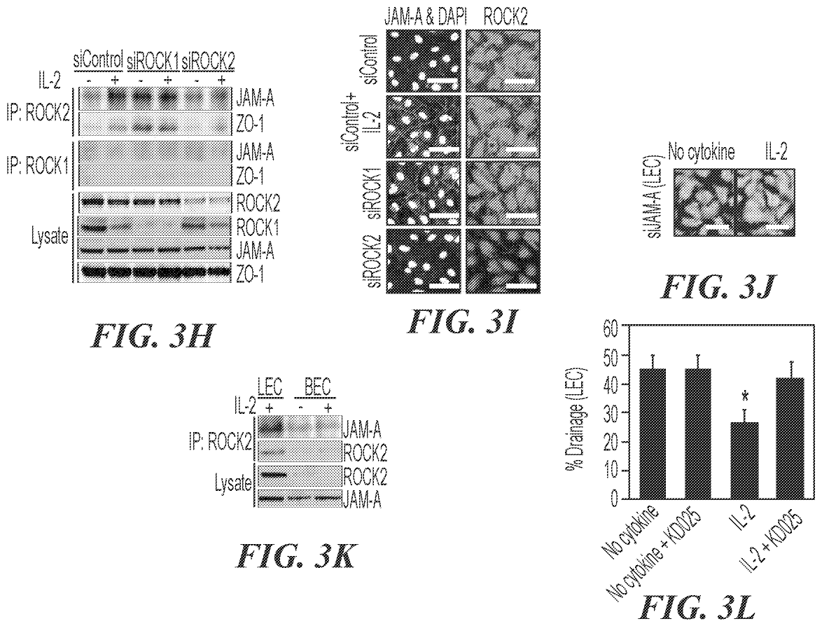

The data provided herein shows that ROCK2 is selectively expressed in LECs (FIGS. 3A and 3B) and that loss of ROCK2 expression ROCK2 loosens adherens junctions (FIG. 3E). Together, these data suggest that inhibition of ROCK2 can normalize tight adherens junctions to support lymphatic drainage. Thus, some aspects of the present disclosure provide methods that comprise contacting dermal lymphatic endothelial cells with a selective ROCK2 inhibitor in an amount effective for preventing formation of tight lymphatic junctions.

Additional studies described herein show that a ROCK2-specific small molecular inhibitor, KD025, reversed tightening of the LEC junctions (data not shown) and impaired drainage (FIG. 3L) against IL-2 challenge. Taken together, the data show that ROCK2 tightens lymphatic junctions when inflammatory signals are present. Thus, some aspects of the present disclosure provide methods that comprise contacting a tight lymphatic junction with a selective ROCK2 inhibitor in an amount effective for improving lymphatic drainage at the lymphatic junction, relative to a control. In some embodiments, the control is a measure of lymphatic drainage at the lymphatic junction before contact with the selective ROCK2 inhibitor.

### Impaired Lymphatic Drainage and Edema

Edema is an accumulation of fluid in the interstitial space that occurs as the capillary filtration exceeds the limits of lymphatic drainage, producing noticeable clinical signs and symptoms (Trayes K P et al. Am Fam Physician. 2013; 88(2):102-110). The chronic accumulation of edema in one or both lower extremities often indicates venous insufficiency, especially in the presence of dependent edema and hemosiderin deposition. Impaired lymphatic drainage results from a blockage/obstruction of a lymphatic vessel that slows or prevents circulation of lymphatic fluid. A “condition associated with impaired lymphatic drainage” is a condition that develops as a result of impaired lymphatic drainage. Impaired lymphatic drainage may lead to, for example, edema, such as lymphedema. Thus, edema (e.g., lymphedema) is a non-limiting example of a condition associated with impaired lymphatic drainage. Causes of impaired lymphatic drainage (e.g., lymphedema) include, for example, the removal of or damage to the lymph nodes (e.g., during cancer treatment), obstructions due to a benign or malignant tumor growth, physical trauma, surgery, obesity, infection of the lymph nodes, chronic venous insufficiency, or malformation of the lymph nodes and lymphatic system during development. Edemas and lymphedemas include, by way of non-limiting example, pulmonary, facial, mastectomy, limb, cerebral, inguinal, and cardiac whether resulting from surgical or postoperative sequelae or other inflammatory, cardiovascular, or cancer related processes.

Almost every organ has organ-residing lymphatics, and the lymphatics drain not only fluid, but lipids, immune cells, and metabolic wastes. Thus, other examples of impaired lymphatic drainage include lipedema (abnormal lipid/fat deposition), obesity and metabolic diseases (gut lymphatics ‘lacteal’ dysfunction), CNS degeneration (brain lymphatic dysfunction and waste deposition), and cellulitis (frequent skin infection owing to the impaired lymphatic drainage of dendritic cells under infection).

In some embodiments, a subject who has impaired lymphatic drainage develops lymphedema. Lymphedema is abnormal swelling that occurs, for example, in an arm, leg, breast, or torso when there is an impairment to the lymphatic system (see, e.g., Grada A A et al. Lymphedema 2017; 77(6): 1009-1020). Lymphedema can be either primary or secondary. Primary lymphedema occurs in the absence of a precipitating disease or condition (e.g., it can be hereditary), and secondary lymphedema is caused by another disease or condition. Causes of secondary lymphedema include, but are not limited to, surgery (e.g., removal of or injury to lymph nodes and lymph vessels), radiation treatment (e.g., radiation can cause scarring and inflammation of lymph nodes or lymph vessels), cancer (e.g., if cancer cells block lymphatic vessels, lymphedema may result), and/or infection (e.g., an infection of the lymph nodes or parasites can restrict the flow of lymph fluid). In some embodiments, a subject has primary lymphedema. In other embodiments, a subject has secondary lymphedema.

Symptoms of impaired lymphatic drainage (e.g., lymphedema) include, for example, swelling of part or all of an arm or leg, including fingers or toes, a feeling of heaviness or tightness, restricted range of motion, aching or discomfort, recurring infections, and/or hardening and thickening of the skin (fibrosis). In some embodiments, a selective ROCK2 inhibitor is administered in an amount effective for alleviating a symptom (e.g., reducing the severity of the symptom) associated with impaired lymphatic drainage. Alleviation of a symptom can be determined by the subject and/or a medical professional (e.g., an attending physician). For example, a selective ROCK2 inhibitor may be administered in an amount effective for reducing (e.g., by at least 10%, 25%, 50%, or 75%) or eliminating swelling of a body part (e.g., arm or leg) of the subject, relative to a control. In some embodiments, the control is the extent of swelling (e.g., volume of fluid) in a particular body part, prior to administering the selective ROCK2 inhibitor.

It should be understood that a subject having a condition associated with impaired lymphatic drainage is a subject who exhibits a symptom of impaired lymphatic drainage and/or has been diagnosed (e.g., by a medical professional) with a condition associated with impaired lymphatic drainage (e.g., lymphedema). A subject at risk of having a condition associated with impaired lymphatic drainage is a subject who has undergone or who will undergo (e.g., with 1 month to 6 months) surgery and/or radiation treatment, or a subject who has cancer or an infection (e.g., in or near a lymph node).

### Selective ROCK2 Inhibitors

ROCK2 (e.g., UniProtKB 075116) is a member of the Rho-associated protein kinase family. It is a key regulator of actin cytoskeleton and cell polarity and is involved in the regulation of smooth muscle contraction, actin cytoskeleton organization, stress fiber and focal adhesion formation, neurite retraction, cell adhesion, and motility (see, e.g., Kawano Y. et al. J. Cell Biol. 1999; 147: 1023-1038; Sebbagh M. et al. J. Exp. Med. 2005; 201: 465-471; Tanaka T. et al. J. Biol. Chem. 2006; 281: 15320-15329; Ma Z. et al. Mol. Cell. Biol. 2006; 26: 9016-9034; Wang Y. et al. Circ. Res. 2009; 104: 531-540; Lock F. et al. PLoS ONE 2009; 4: E8190-E8190; Wang H. et al. Cancer Res. 2011; 71: 68-77; and Herskowitz J. et al. J. Biol. Chem. 2011; 286:6117-6127).

A selective ROCK2 inhibitor, as used herein, is an agent (e.g., polypeptide, polynucleotide, or chemical compound (e.g., small molecule)) that has an at least 2-fold lower (e.g., 2 to 10-fold lower) IC₅₀ for ROCK2, relative to ROCK1. In some embodiments, a selective ROCK2 inhibitor has an at least 5-fold, or an at least 10-fold lower IC₅₀ for ROCK2, relative to ROCK1. In some embodiments, a selective ROCK2 inhibitor can inhibit ROCK2 gene expression, ROCK2 protein production, and/or ROCK2 protein activity, without substantially inhibiting gene expression, protein production, and/or protein function of other ROCK family members, for example, ROCK1 (e.g., UniProtKB Q13464). Use of a selective ROCK2 inhibitor, in some embodiments, circumvents the negative side effects associated with ROCK1 and/or pan-ROCK) inhibition, such as hypotension.

Inhibition encompasses the prevention of gene expression and/or protein activity such that gene expression, protein production, and/or protein activity cannot be detected using standard gene expression, protein expression, and/or protein activity assays. Inhibition also encompasses a reduction of gene expression, protein expression, and/or protein activity such that gene expression, protein production, and/or protein activity can be detected using standard gene expression, protein production, and/or protein activity assays at a level that is lower than a control. For example, ROCK2 gene expression, ROCK2 protein production, and/or ROCK2 protein activity may be reduced by at least 10% in the presence of a selective ROCK2 inhibitor (e.g., a small molecule inhibitor), relative to a control. In some embodiments, ROCK2 gene expression, ROCK2 protein production, and/or ROCK2 protein activity is reduced by at least 20%, at least 30%, at least 40%, at least 50%, at last 60%, at least 70%, at least 80%, at least 90%, or 100% in the presence of a selective ROCK2 inhibitor (e.g., a small molecule inhibitor), relative to a control. In some embodiments, ROCK2 gene expression, ROCK2 protein production, and/or ROCK2 protein activity is reduced by 10%-100% in the presence of a selective ROCK2 inhibitor (e.g., a small molecule inhibitor), relative to a control. For example, ROCK2 gene expression, ROCK2 protein production, and/or ROCK2 protein activity may be reduced by 20%-100%, 30%-100%, 40%-100%, 50%-100%, 60%-100%, 70%-100%, 80%-100%, 90%-100%, 10%-90%, 20%-90%, 30%-90%, 40%-90%, 50%-90%, 60%-90%, 70%-90%, 80%-90%, 10%-80%, 20%-80%, 30%-80%, 40%-80%, 50%-80%, 60%-80%, or 70%-80% in the presence of a selective ROCK2 inhibitor (e.g., a small molecule inhibitor), relative to a control. In some embodiments, the control is ROCK2 gene expression in the absence of the ROCK2 inhibitor (all other conditions being the same). In some embodiments, the control is ROCK2 protein production in the absence of the ROCK2 inhibitor (all other conditions being the same). In some embodiments, the control is ROCK2 protein activity in the absence of the ROCK2 inhibitor (all other conditions being the same).

Methods for measuring gene expression levels, protein expression levels, and/or protein expression levels are known. Non-limiting examples of gene expression assays include in situ hybridization and polymerase chain reaction. Non-limiting examples of protein production and/or activity assays include antibody-based assays, such as an enzyme-linked immunosorbent assay (ELISA), immunostaining, immunoblotting, and flow cytometry.

An assay for measuring gene expression levels, protein expression levels, and/or protein expression levels, in some embodiments, is performed on a sample obtained from a subject (e.g., who has been administered a selective ROCK2 inhibitor). The sample may be a blood sample or a tissue sample. Other biological samples may be used.

In some embodiments, a selective ROCK2 inhibitor binds to ROCK2 and inhibits ROCK2 serine/threonine kinase activity. In some embodiments, a selective ROCK2 inhibitor binds to ROCK2 and inhibits ROCK2 serine/threonine kinase activity by at least 10%, relative to a control. For example, a selective ROCK2 inhibitor may bind to ROCK2 and inhibit ROCK2 serine/threonine kinase activity by at least 20%, at least 30%, at least 40%, at least 50%, at last 60%, at least 70%, at least 80%, at least 90%, or 100%, relative to a control. In some embodiments, a selective ROCK2 inhibitor binds to ROCK2 and inhibits ROCK2 serine/threonine kinase activity by 10%-100%, relative to a control. For example, a selective ROCK2 inhibitor may bind to ROCK2 and inhibit ROCK2 serine/threonine kinase activity by 20%-100%, 30%-100%, 40%-100%, 50%-100%, 60%-100%, 70%-100%, 80%-100% a, 90%-100%, 10%-90%, 20%-90%, 30%-90%, 40%-90%, 50%-90%, 60%-90%, 70%-90%, 80%-90%, 10%-80%, 20%-80%, 30%-80%, 40%-80%, 50%-80%, 60%-80%, or 70%-80%, relative to a control. In some embodiments, the control is ROCK2 serine/threonine kinase activity in the absence of the ROCK2 inhibitor (all other conditions being the same).

Non-limiting examples of polynucleotides that selectively inhibit ROCK2 gene and/or ROCK2 protein expression include antisense molecules and RNA interference (RNAi) molecules that specifically bind to a ROCK2 gene (e.g., to a promoter, coding region, non-coding region, or other regulatory region of the ROCK2 gene). Examples of RNAi molecules include, but are not limited to, small interfering RNA (siRNA), short hairpin RNA (shRNA), and micro RNA (miRNA).

Non-limiting examples of polypeptides that selectively inhibit ROCK2 protein activity include antibodies, aptamers, and other protein-binding molecules. The term “antibody” encompasses whole antibodies and antibody fragments (e.g., single chain variable fragments (scFvs)). In some embodiments, a selective ROCK2 inhibitor is an antibody (e.g., a monoclonal antibody) that binds specifically to ROCK2.

Non-limiting examples of chemical compounds that selectively inhibit ROCK2 gene expression and/or ROCK2 protein activity include small molecules (e.g., less than 10 kDa). Small molecule inhibitors, for example, may bind ROCK2 with an IC₅₀ value of 50 nM-150 nM (e.g., 50 nM, 60 nM, 70 nM, 80 nM, 90 nM, 100 nM, 110 nM, 120 nM, 130 nM, 140 nM, or 150 nM. In some embodiments, a small molecule inhibitor binds ROCK1 with an IC₅₀ value of greater than 200 nM. For example, a small molecule inhibitor binds ROCK1 with an IC₅₀ value of greater than 500 nM, greater than 1000 nM, or greater than 10,000 nM. In some embodiments, a small molecule inhibitor binds ROCK1 with an IC₅₀ value of 20,000 nM to 25,000 nM.

In some embodiments, the selective ROCK2 inhibitor is 2-[3-[4-(1H-indazol-5-ylamino)-2-quinazolinyl]phenoxy]-N-(1-methylethyl)-acetamide (KD025). KD025 is also referred to as SLx-2119 (Boerma et al. Blood Coagul Fibrinolysis 2009; 19(7): 709-718). KD025 inhibits ROCK2 by binding and suppressing its serine/threonine kinase activity. This inhibits ROCK2-mediated signaling pathways. Surprisingly, as discussed herein, KD025 alone was sufficient to reduce lymphatic drainage in an in vitro model system (see FIG. 3L). Thus, in some embodiments, the present disclosure provides a method of administering KD025 to a subject having or at risk of a condition associated with impaired lymphatic drainage. Non-limiting examples of other ROCK2 inhibitors include SR-3677 (TOCHRIS®) and Chroman 1 (MCE®).

Other non-limiting examples of selective ROCK2 inhibitors are provided in Table 1 and in International Publication Number WO 2019/145729 A1, incorporated by reference herein in its entirety. In some embodiments, the selective ROCK2 small molecule inhibitor is a compound of formula (I) (i.e., including any subgenera or species thereof) disclosed in International Publication Number WO 2019/145729 A1, incorporated by reference herein in its entirety; or a pharmaceutically acceptable salt, stereoisomer, tautomer, isotopically labeled derivative, solvate, hydrate, polymorph, co-crystal, or prodrug thereof. In some embodiments, the selective ROCK2 small molecule inhibitor is a compound of formula (I) (i.e., including any subgenera or species thereof) disclosed in International Publication Number WO 2019/145729 A1, incorporated by reference herein in its entirety; or a pharmaceutically acceptable salt thereof.

For instance, in some embodiments, the selective ROCK2 small molecule inhibitor is a compound of formula (I), or a pharmaceutically acceptable salt thereof:

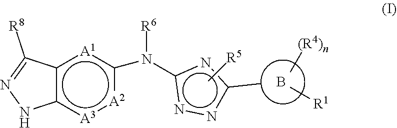

wherein:
A¹, A² and A³ are each independently selected from CH, CR⁷ and N;
B represents a 5 to 10 membered carbocyclic ring system or a 5 to 10 membered heterocyclic ring system;
R¹ is L-R², wherein L is a bond or -L¹-L²-;
L¹ is selected from a bond, —(CR^{A}R^{B})₁₋₃—, —O(CR^{A}R^{B})₁₋₃—, —(CR^{A}R^{B})₀₋₃O—, and NR^{C}(CR^{A}R^{B})₁₋₃—, and L² is selected from a bond, —(CR^{A}R^{B})₁₋₃—, —O—, —NR^{D}—, —C(O)NR^{D}—. —NR^{D}C(O)—, —C(O)O—, —OC(O)—, —C(O)—, —S(O)₂NR^{D}—, —NR^{D}S(O)₂—, —S(O)₂—, —S(O)(NR^{D})—, —NR^{D}C(O)NR^{E}—, —OC(O)NR^{D}—, and —C(O)NR^{D}S(O)₂—;
R² is selected from —H, —CN, C₁₋₆ alkyl, C₁₋₆ haloalkyl, C₁₋₆ alkyl substituted with —OR^{F}, C₁₋₆ alkyl substituted with —NR^{F}R^{G}, C₁₋₄ haloalkyl substituted with —OR^{F}, C₃₋₈ cycloalkyl substituted with —OH, C₁₋₄ alkyl substituted with 3 to 8 membered heterocycloalkyl, C₁₋₄ alkyl substituted with 6 membered heteroaryl, —(CR^{H}R^{I})₁₋₃OR^{F}, —(CR^{H}R^{I})₁₋₃NR^{F}R^{G}, —(CR^{N}R^{O})₁₋₃C(O)OR^{F}, —(CR^{N}R^{O})₁₋₃C(O)NR^{F}R^{G}, C₃₋₁₀ carbocyclic ring system, and 3 to 10 membered heterocyclic ring system, wherein the carbocyclic ring system or heterocyclic ring system is unsubstituted or substituted with ═O, —NR^{F}R^{G}, —C(O)R^{F}, halogen, —CN, C₁₋₄ alkyl, C₁₋₄ haloalkyl, or C₁₋₄ alkyl substituted with —OR^{F};
R⁴ is independently selected at each occurrence from halogen, C₁₋₄ alkyl, C₁₋₆ haloalkyl, —CN, —OR^{J}, ═O, C₁₋₄ alkyl substituted with —OR^{J}, —NR^{J}R^{K}, C₁₋₄ alkyl substituted with —NR^{J}R^{K}, C₃₋₈ cycloalkyl, C₁₋₄ alkyl substituted with C₃₋₈ cycloalkyl, 3 to 8 membered heterocycloalkyl, and C₁₋₄ alkyl substituted with 3 to 8 membered heterocycloalkyl;
R⁵ is selected from —H, C₁₋₄alkyl, C₁₋₄ alkyl substituted with —OR^{L}, C₁₋₄alkyl substituted with —NR^{L}R^{L}, C₃₋₈ cycloalkyl, substituted or unsubstituted phenyl, 3 to 8 membered heterocycloalkyl, C₁₋₄ alkyl substituted with C₃₋₈ cycloalkyl, C₁₋₄alkyl substituted with 3 to 8 membered heterocycloalkyl, and substituted or unsubstituted 5 or 6 membered heteroaryl, wherein the phenyl or heteroaryl group may be substituted by 1 or 2 R⁹;
R⁶ is selected from —H and C₁₋₄ alkyl;
R⁷ is selected from —H, halogen, —OR^{M}, C₁₋₄ alkyl, C₁₋₆ haloalkyl, C₁₋₄ alkenyl, —CN, and C₃₋₈ cycloalkyl;
R⁸ is selected from —H, halogen, C₁₋₄ alkyl, C₁₋₆ haloalkyl, —CN, and C₃₋₈ cycloalkyl;
R⁹ is selected from halogen and C₁₋₄ alkyl;
n is 0, 1, or 2;
R^{A} and R^{B} are selected from —H, C₁₋₄alkyl, and C₁₋₄haloalkyl, or R^{A} and R^{B} together with the atom to which they are attached form a 3 to 6 membered cycloalkyl ring or a 3 to 6 membered heterocycloalkyl ring;
R^{C}, R^{D}, R^{E}, R^{F} and R^{G} are each independently selected from H, C₁₋₄ alkyl, and C₁₋₄ haloalkyl;
R^{H} and R^{I} are each —H except one pair of R^{H} and R^{I} on the same carbon atom, together with that carbon atom, form a 3 to 6 membered cycloalkyl ring or a 3 to 6 membered heterocycloalkyl ring; and
R^{J}, R^{K}, R^{L}, R^{M}, R^{N} and R^{O} are each independently at each occurrence selected from H and C₁₋₄ alkyl. Additional embodiments, including subgenera and species of formula (I), are provided in International Publication Number WO 2019/145729 A1, incorporated by reference herein in its entirety.

In some embodiments, the selective ROCK2 small molecule inhibitor is a compound of formula (I), or a pharmaceutically acceptable salt thereof:

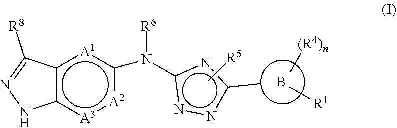

wherein:
A¹, A² and A³ are each independently selected from CH, CR⁷ and N;
B represents a 5 to 10 membered carbocyclic ring system or a 5 to 10 membered heterocyclic ring system;
R¹ is L-R², wherein L is -L¹-L²-;
L¹ is selected from a bond, —(CR^{A}R^{B})₁₋₃, —O(CR^{A}R^{B})₁₋₃—, —(CR^{A}R^{B})₀₋₃O—, and NR^{C}(CR^{A}R^{B})₁₋₃—, and L² is selected from a bond, —(CR^{A}R^{B})₁₋₃—, —O—, —NR^{D}—, —C(O)NR^{D}—, —NR^{D}C(O)—, —C(O)O—, —OC(O)—, —C(O)—, —S(O)₂NR^{D}—, —NR^{D}S(O)₂—, —S(O)₂—, —S(O)(NR^{D})—, —NR^{D}C(O)NR^{E}—, —OC(O)NR^{D}—, and —C(O)NR^{D}S(O)₂—;
R² is selected from —H, —CN, C₁₋₆ alkyl, C₁₋₆ haloalkyl, C₁₋₆ alkyl substituted with —OR^{F}, C₁₋₆ alkyl substituted with —NR^{F}R^{G}, C₃₋₈ cycloalkyl substituted with —OH, C₁₋₄ alkyl substituted with 3 to 8 membered heterocycloalkyl, C₁₋₄ alkyl substituted with 6 membered heteroaryl, —(CR^{H}R^{I})₁₋₃OR^{F}, —(CR^{H}R^{I})₁₋₃NR^{F}R^{G}, C₃₋₁₀ carbocyclic ring system, and 3 to 10 membered heterocyclic ring system, wherein the carbocyclic ring system or heterocyclic ring system is unsubstituted or substituted with ═O, —NR^{F}R^{G}, halogen, C₁₋₄ alkyl, or C₁₋₄ alkyl substituted with —OR^{F};
R⁴ is selected from halogen, C₁₋₄ alkyl, C₁₋₆ haloalkyl, —CN, —OR^{J}, ═O, C₁₋₄ alkyl substituted with —OR^{J}, —NR^{J}R^{K}, C₁₋₄ alkyl substituted with —NR^{J}R^{K}, C₃₋₈ cycloalkyl, C₁₋₄ alkyl substituted with C₃₋₈ cycloalkyl, 3 to 8 membered heterocycloalkyl, and C₁₋₄ alkyl substituted with 3 to 8 membered heterocycloalkyl;
R⁵ is selected from —H, C₁₋₄ alkyl, C₁₋₄ alkyl substituted with —OR^{L}, C₃₋₈ cycloalkyl, substituted or unsubstituted phenyl, 3 to 8 membered heterocycloalkyl, C₁₋₄ alkyl substituted with C₃₋₈ cycloalkyl, C₁₋₄ alkyl substituted with 3 to 8 membered heterocycloalkyl, and substituted or unsubstituted 5 or 6 membered heteroaryl, wherein the phenyl or heteroaryl group may be substituted by 1 or 2 R⁹;
R⁶ is selected from —H and C₁₋₄ alkyl;
R⁷ is selected from —H, halogen, —OR^{M}, C₁₋₄ alkyl, C₁₋₆ haloalkyl, —CN, and C₃₋₈, cycloalkyl;
R⁸ is selected from —H, halogen, C₁₋₄ alkyl, C₁₋₆ haloalkyl, —CN, and C₃₋₈ cycloalkyl;
R⁹ is selected from halogen and C₁₋₄ alkyl;
n is 0, 1, or 2;
R^{A} and R^{B} are selected from —H, C₁₋₄ alkyl, and C₁₋₄ haloalkyl, or R^{A} and R^{B} together with the atom to which they are attached form a 3 to 6 membered cycloalkyl ring or a 3 to 6 membered heterocycloalkyl ring;
R^{C}, R^{D}, R^{E}, R^{F} and R^{G} are each independently selected from −1 and C₁₋₄ alkyl;
R^{H} and R^{I} are each H except one pair of R^{H} and R^{I} on the same carbon atom, together with that carbon atom, form a 3 to 6 membered cycloalkyl ring or a 3 to 6 membered heterocycloalkyl ring; and R^{J}, R^{K}, R^{L} and R^{M} are each independently at each occurrence selected from —H and C₁₋₄ alkyl. Additional embodiments, including subgenera and species of formula (I), are provided in International Publication Number WO 2019/145729 A1, incorporated by reference herein in its entirety.

In some embodiments, the selective ROCK2 small molecule inhibitor comprises a compound selected from Compounds 1-317 of Table 1. In some embodiments, the selective ROCK2 small molecule inhibitor is selected from Compounds 1-317 of Table 1. In some embodiments, the selective ROCK2 small molecule inhibitor is any one of Compounds 1-317 of Table 1, or a pharmaceutically acceptable salt, stereoisomer, tautomer, isotopically labeled derivative, solvate, hydrate, polymorph, co-crystal, or prodrug thereof. In some embodiments, the selective ROCK2 small molecule inhibitor is any one of Compounds 1-317 of Table 1, or a pharmaceutically acceptable salt thereof.

TABLE 1
Examples of Selective ROCK2 Inhibitors

| Compound | Structure |
|---|---|
| 1 | 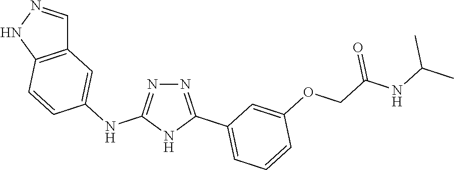 [SMILES: CC(C)NC(=O)COc1cccc(-c2nnc(Nc3ccc4[nH]ncc4c3)[nH]2)c1] |
| 2 | 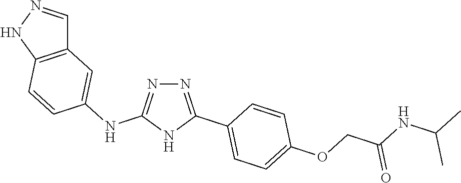 [SMILES: CC(C)NC(=O)COc1ccc(-c2nnc(Nc3ccc4[nH]ncc4c3)[nH]2)cc1] |
| 3 | 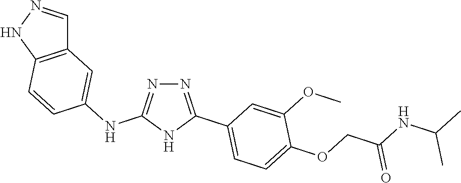 [SMILES: COc1cc(-c2nnc(Nc3ccc4[nH]ncc4c3)[nH]2)ccc1OCC(=O)NC(C)C] |
| 4 | 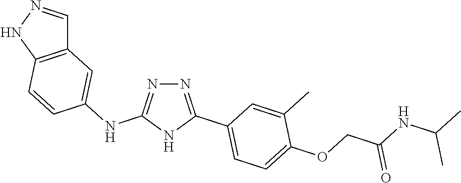 [SMILES: Cc1cc(-c2nnc(Nc3ccc4[nH]ncc4c3)[nH]2)ccc1OCC(=O)NC(C)C] |
| 5 | 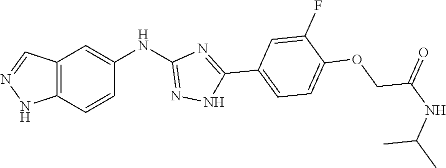 [SMILES: CC(C)NC(=O)COc1ccc(-c2nc(Nc3ccc4[nH]ncc4c3)n[nH]2)cc1F] |
| 6 | 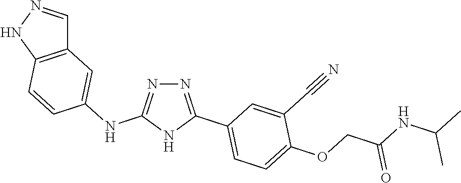 [SMILES: CC(C)NC(=O)COc1ccc(-c2nnc(Nc3ccc4[nH]ncc4c3)[nH]2)cc1C#N] |
| 7 | 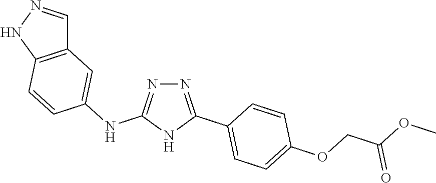 [SMILES: COC(=O)COc1ccc(-c2nnc(Nc3ccc4[nH]ncc4c3)[nH]2)cc1] |
| 8 | 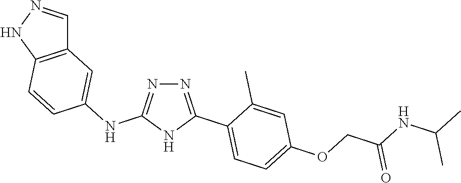 [SMILES: Cc1cc(OCC(=O)NC(C)C)ccc1-c1nnc(Nc2ccc3[nH]ncc3c2)[nH]1] |
| 9 | 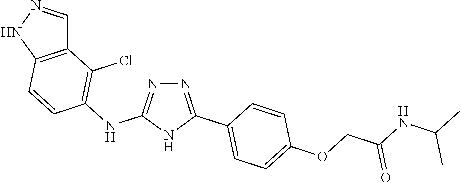 [SMILES: CC(C)NC(=O)COc1ccc(-c2nnc(Nc3ccc4[nH]ncc4c3Cl)[nH]2)cc1] |
| 10 | 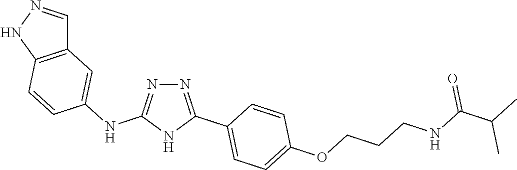 [SMILES: CC(C)C(=O)NCCCOc1ccc(-c2nnc(Nc3ccc4[nH]ncc4c3)[nH]2)cc1] |
| 11 | 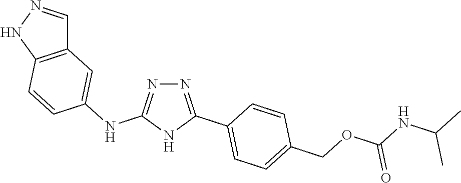 [SMILES: CC(C)NC(=O)OCc1ccc(-c2nnc(Nc3ccc4[nH]ncc4c3)[nH]2)cc1] |
| 12 | 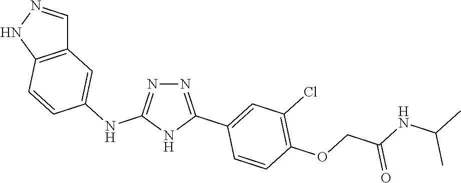 [SMILES: CC(C)NC(=O)COc1ccc(-c2nnc(Nc3ccc4[nH]ncc4c3)[nH]2)cc1Cl] |
| 13 | 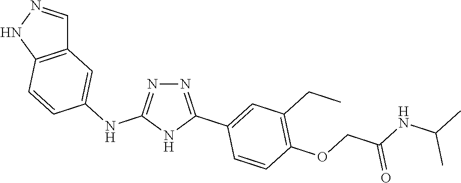 [SMILES: CCc1cc(-c2nnc(Nc3ccc4[nH]ncc4c3)[nH]2)ccc1OCC(=O)NC(C)C] |
| 14 | 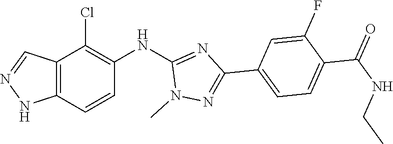 [SMILES: CCNC(=O)c1ccc(-c2nc(Nc3ccc4[nH]ncc4c3Cl)n(C)n2)cc1F] |
| 15 | 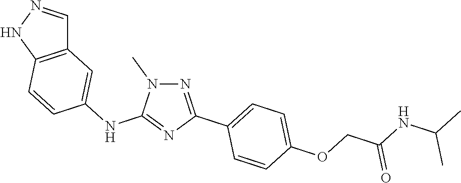 [SMILES: CC(C)NC(=O)COc1ccc(-c2nc(Nc3ccc4[nH]ncc4c3)n(C)n2)cc1] |
| 16 | 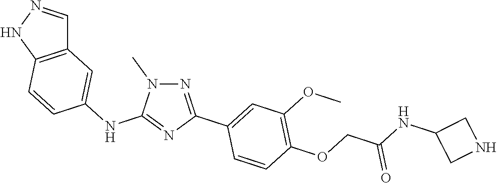 [SMILES: COc1cc(-c2nc(Nc3ccc4[nH]ncc4c3)n(C)n2)ccc1OCC(=O)NC1CNC1] |
| 17 | 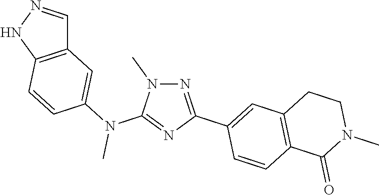 [SMILES: CN1CCc2cc(-c3nc(N(C)c4ccc5[nH]ncc5c4)n(C)n3)ccc2C1=O] |
| 18 | 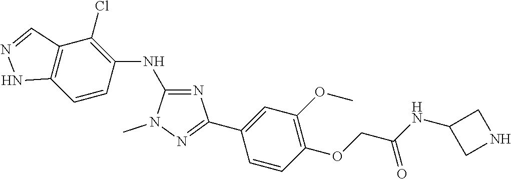 [SMILES: COc1cc(-c2nc(Nc3ccc4[nH]ncc4c3Cl)n(C)n2)ccc1OCC(=O)NC1CNC1] |
| 19 | 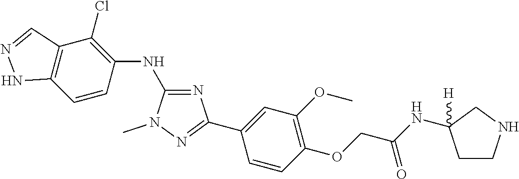 [SMILES: COc1cc(-c2nc(Nc3ccc4[nH]ncc4c3Cl)n(C)n2)ccc1OCC(=O)NC1CCNC1] |
| 20 | 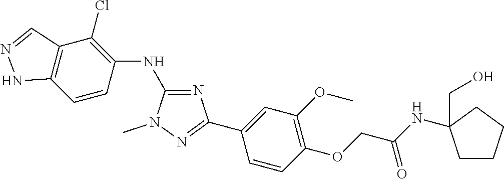 [SMILES: COc1cc(-c2nc(Nc3ccc4[nH]ncc4c3Cl)n(C)n2)ccc1OCC(=O)NC1(CO)CCCC1] |
| 21 | 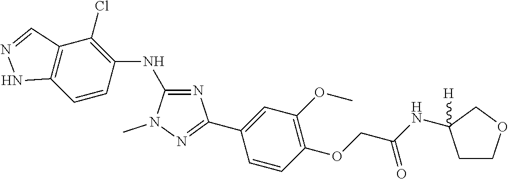 [SMILES: COc1cc(-c2nc(Nc3ccc4[nH]ncc4c3Cl)n(C)n2)ccc1OCC(=O)NC1CCOC1] |
| 22 | 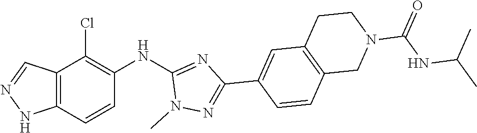 [SMILES: CC(C)NC(=O)N1CCc2cc(-c3nc(Nc4ccc5[nH]ncc5c4Cl)n(C)n3)ccc2C1] |
| 23 | 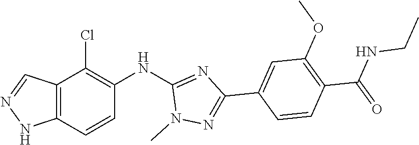 [SMILES: CCNC(=O)c1ccc(-c2nc(Nc3ccc4[nH]ncc4c3Cl)n(C)n2)cc1OC] |
| 24 | 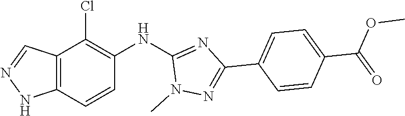 [SMILES: COC(=O)c1ccc(-c2nc(Nc3ccc4[nH]ncc4c3Cl)n(C)n2)cc1] |
| 25 | 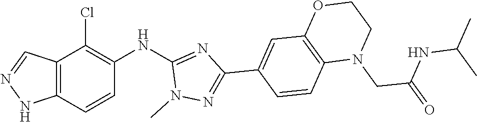 [SMILES: CC(C)NC(=O)CN1CCOc2cc(-c3nc(Nc4ccc5[nH]ncc5c4Cl)n(C)n3)ccc21] |
| 26 | 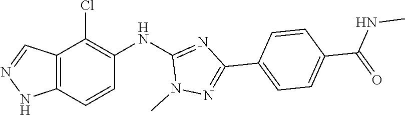 [SMILES: CNC(=O)c1ccc(-c2nc(Nc3ccc4[nH]ncc4c3Cl)n(C)n2)cc1] |
| 27 | 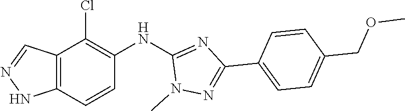 [SMILES: COCc1ccc(-c2nc(Nc3ccc4[nH]ncc4c3Cl)n(C)n2)cc1] |
| 28 | 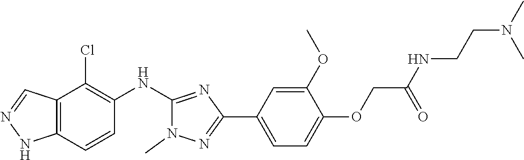 [SMILES: COc1cc(-c2nc(Nc3ccc4[nH]ncc4c3Cl)n(C)n2)ccc1OCC(=O)NCCN(C)C] |
| 29 | 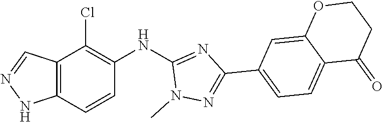 [SMILES: Cn1nc(-c2ccc3c(c2)OCCC3=O)nc1Nc1ccc2[nH]ncc2c1Cl] |
| 30 | 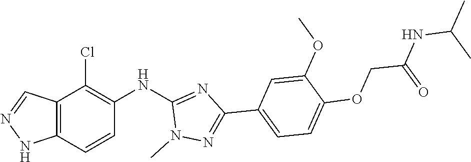 [SMILES: COc1cc(-c2nc(Nc3ccc4[nH]ncc4c3Cl)n(C)n2)ccc1OCC(=O)NC(C)C] |
| 31 | 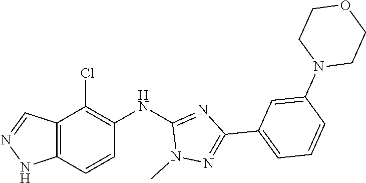 [SMILES: Cn1nc(-c2cccc(N3CCOCC3)c2)nc1Nc1ccc2[nH]ncc2c1Cl] |
| 32 | 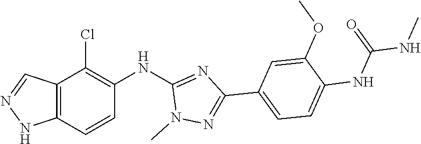 [SMILES: CNC(=O)Nc1ccc(-c2nc(Nc3ccc4[nH]ncc4c3Cl)n(C)n2)cc1OC] |
| 33 | 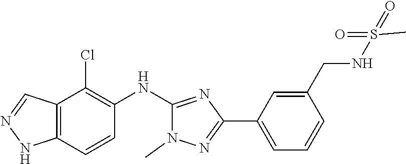 [SMILES: Cn1nc(-c2cccc(CNS(C)(=O)=O)c2)nc1Nc1ccc2[nH]ncc2c1Cl] |
| 34 | 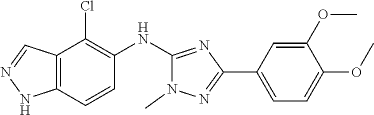 [SMILES: COc1ccc(-c2nc(Nc3ccc4[nH]ncc4c3Cl)n(C)n2)cc1OC] |
| 35 | 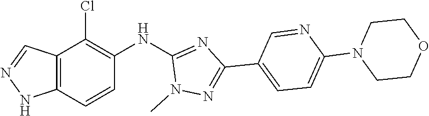 [SMILES: Cn1nc(-c2ccc(N3CCOCC3)nc2)nc1Nc1ccc2[nH]ncc2c1Cl] |
| 36 | 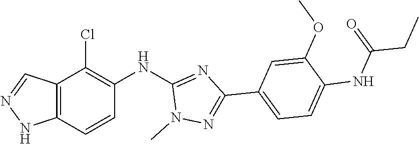 [SMILES: CCC(=O)Nc1ccc(-c2nc(Nc3ccc4[nH]ncc4c3Cl)n(C)n2)cc1OC] |
| 37 | 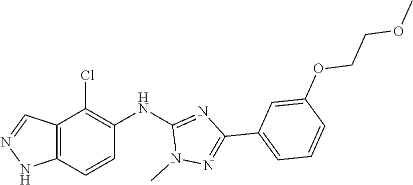 [SMILES: COCCOc1cccc(-c2nc(Nc3ccc4[nH]ncc4c3Cl)n(C)n2)c1] |
| 38 | 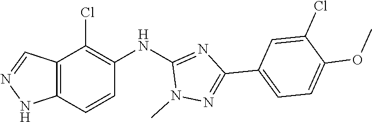 [SMILES: COc1ccc(-c2nc(Nc3ccc4[nH]ncc4c3Cl)n(C)n2)cc1Cl] |
| 39 | 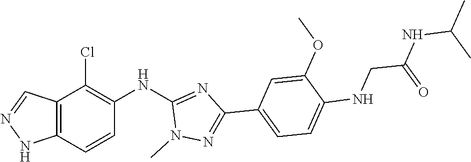 [SMILES: COc1cc(-c2nc(Nc3ccc4[nH]ncc4c3Cl)n(C)n2)ccc1NCC(=O)NC(C)C] |
| 40 | 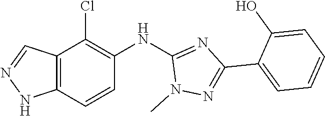 [SMILES: Cn1nc(-c2ccccc2O)nc1Nc1ccc2[nH]ncc2c1Cl] |
| 41 | 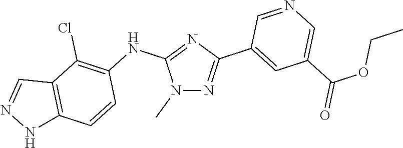 [SMILES: CCOC(=O)c1cncc(-c2nc(Nc3ccc4[nH]ncc4c3Cl)n(C)n2)c1] |
| 42 | 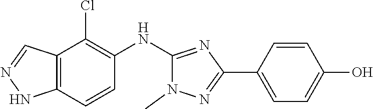 [SMILES: Cn1nc(-c2ccc(O)cc2)nc1Nc1ccc2[nH]ncc2c1Cl] |
| 43 | 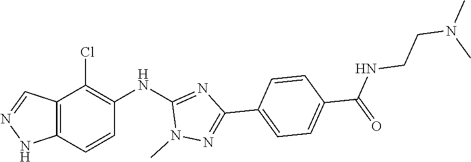 [SMILES: CN(C)CCNC(=O)c1ccc(-c2nc(Nc3ccc4[nH]ncc4c3Cl)n(C)n2)cc1] |
| 44 | 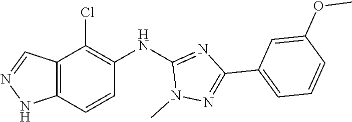 [SMILES: COc1cccc(-c2nc(Nc3ccc4[nH]ncc4c3Cl)n(C)n2)c1] |
| 45 | 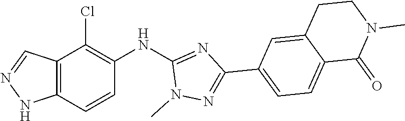 [SMILES: CN1CCc2cc(-c3nc(Nc4ccc5[nH]ncc5c4Cl)n(C)n3)ccc2C1=O] |
| 46 | 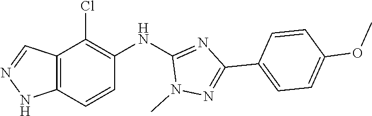 [SMILES: COc1ccc(-c2nc(Nc3ccc4[nH]ncc4c3Cl)n(C)n2)cc1] |
| 47 | 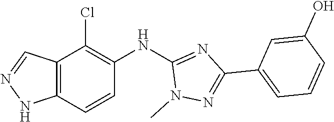 [SMILES: Cn1nc(-c2cccc(O)c2)nc1Nc1ccc2[nH]ncc2c1Cl] |
| 48 | 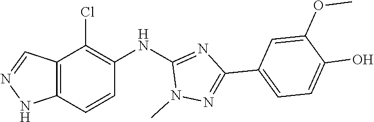 [SMILES: COc1cc(-c2nc(Nc3ccc4[nH]ncc4c3Cl)n(C)n2)ccc1O] |
| 49 | 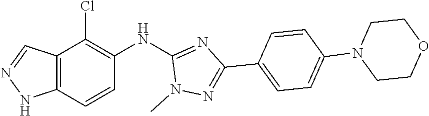 [SMILES: Cn1nc(-c2ccc(N3CCOCC3)cc2)nc1Nc1ccc2[nH]ncc2c1Cl] |
| 50 | 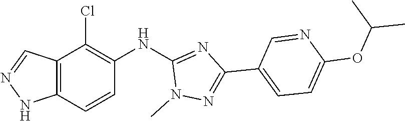 [SMILES: CC(C)Oc1ccc(-c2nc(Nc3ccc4[nH]ncc4c3Cl)n(C)n2)cn1] |
| 51 | 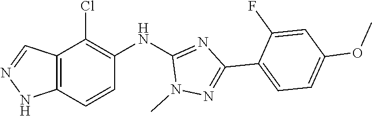 [SMILES: COc1ccc(-c2nc(Nc3ccc4[nH]ncc4c3Cl)n(C)n2)c(F)c1] |
| 52 | 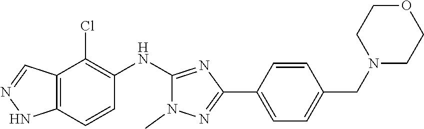 [SMILES: Cn1nc(-c2ccc(CN3CCOCC3)cc2)nc1Nc1ccc2[nH]ncc2c1Cl] |
| 53 | 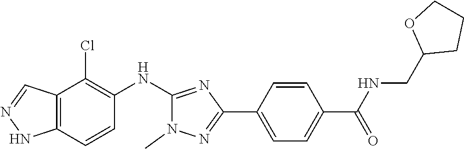 [SMILES: Cn1nc(-c2ccc(C(=O)NCC3CCCO3)cc2)nc1Nc1ccc2[nH]ncc2c1Cl] |
| 54 | 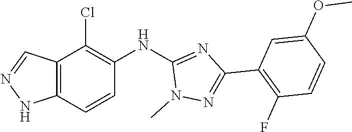 [SMILES: COc1ccc(F)c(-c2nc(Nc3ccc4[nH]ncc4c3Cl)n(C)n2)c1] |
| 55 | 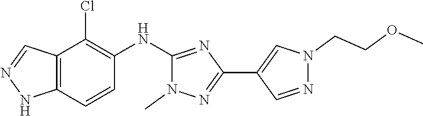 [SMILES: COCCn1cc(-c2nc(Nc3ccc4[nH]ncc4c3Cl)n(C)n2)cn1] |
| 56 | 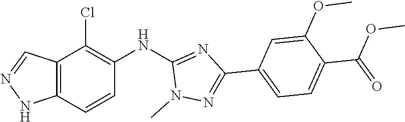 [SMILES: COC(=O)c1ccc(-c2nc(Nc3ccc4[nH]ncc4c3Cl)n(C)n2)cc1OC] |
| 57 | 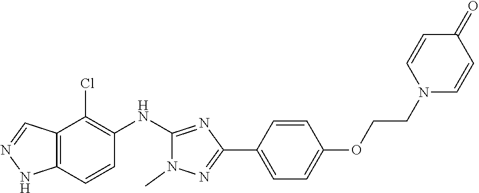 [SMILES: Cn1nc(-c2ccc(OCCn3ccc(=O)cc3)cc2)nc1Nc1ccc2[nH]ncc2c1Cl] |
| 58 | 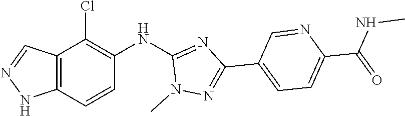 [SMILES: CNC(=O)c1ccc(-c2nc(Nc3ccc4[nH]ncc4c3Cl)n(C)n2)cn1] |
| 59 | 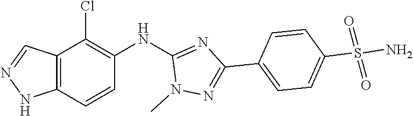 [SMILES: Cn1nc(-c2ccc(S(N)(=O)=O)cc2)nc1Nc1ccc2[nH]ncc2c1Cl] |
| 60 | 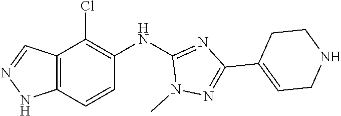 [SMILES: Cn1nc(C2=CCNCC2)nc1Nc1ccc2[nH]ncc2c1Cl] |
| 61 | 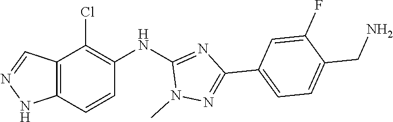 [SMILES: Cn1nc(-c2ccc(CN)c(F)c2)nc1Nc1ccc2[nH]ncc2c1Cl] |
| 62 | 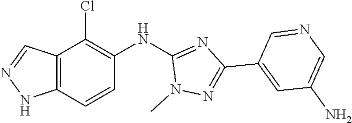 [SMILES: Cn1nc(-c2cncc(N)c2)nc1Nc1ccc2[nH]ncc2c1Cl] |
| 63 | 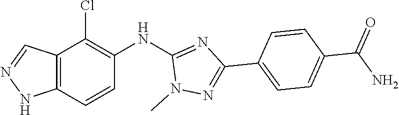 [SMILES: Cn1nc(-c2ccc(C(N)=O)cc2)nc1Nc1ccc2[nH]ncc2c1Cl] |
| 64 | 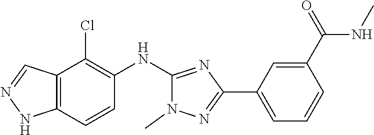 [SMILES: CNC(=O)c1cccc(-c2nc(Nc3ccc4[nH]ncc4c3Cl)n(C)n2)c1] |
| 65 | 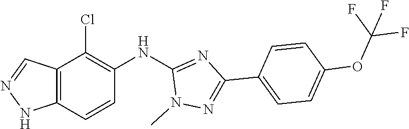 [SMILES: Cn1nc(-c2ccc(OC(F)(F)F)cc2)nc1Nc1ccc2[nH]ncc2c1Cl] |
| 66 | 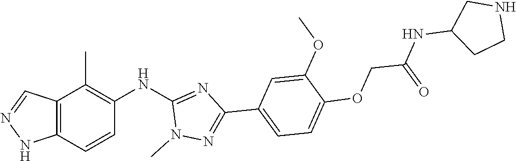 [SMILES: COc1cc(-c2nc(Nc3ccc4[nH]ncc4c3C)n(C)n2)ccc1OCC(=O)NC1CCNC1] |
| 67 | 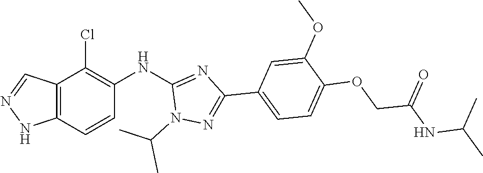 [SMILES: COc1cc(-c2nc(Nc3ccc4[nH]ncc4c3Cl)n(C(C)C)n2)ccc1OCC(=O)NC(C)C] |
| 68 | 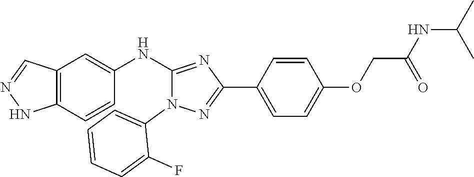 [SMILES: CC(C)NC(=O)COc1ccc(-c2nc(Nc3ccc4[nH]ncc4c3)n(-c3ccccc3F)n2)cc1] |
| 69 | 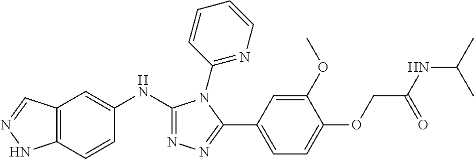 [SMILES: COc1cc(-c2nnc(Nc3ccc4[nH]ncc4c3)n2-c2ccccn2)ccc1OCC(=O)NC(C)C] |
| 70 | 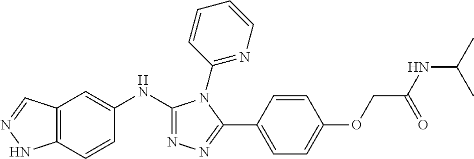 [SMILES: CC(C)NC(=O)COc1ccc(-c2nnc(Nc3ccc4[nH]ncc4c3)n2-c2ccccn2)cc1] |
| 71 | 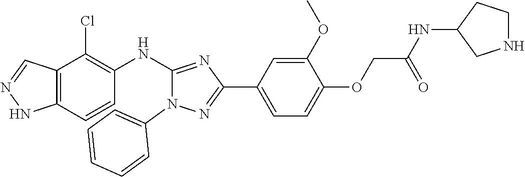 [SMILES: COc1cc(-c2nc(Nc3ccc4[nH]ncc4c3Cl)n(-c3ccccc3)n2)ccc1OCC(=O)NC1CCNC1] |
| 72 | 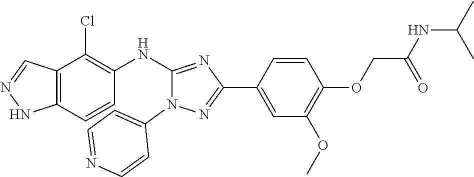 [SMILES: COc1cc(-c2nc(Nc3ccc4[nH]ncc4c3Cl)n(-c3ccncc3)n2)ccc1OCC(=O)NC(C)C] |
| 73 | 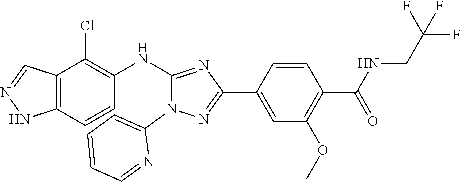 [SMILES: COc1cc(-c2nc(Nc3ccc4[nH]ncc4c3Cl)n(-c3ccccn3)n2)ccc1C(=O)NCC(F)(F)F] |
| 74 | 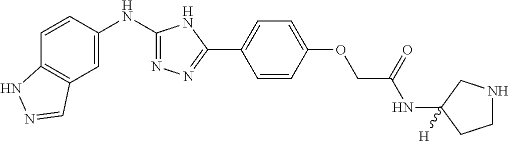 [SMILES: O=C(COc1ccc(-c2nnc(Nc3ccc4[nH]ncc4c3)[nH]2)cc1)NC1CCNC1] |
| 75 | 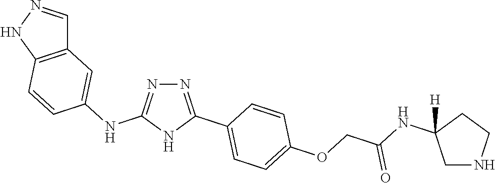 [SMILES: O=C(COc1ccc(-c2nnc(Nc3ccc4[nH]ncc4c3)[nH]2)cc1)N[C@@H]1CCNC1] |
| 76 | 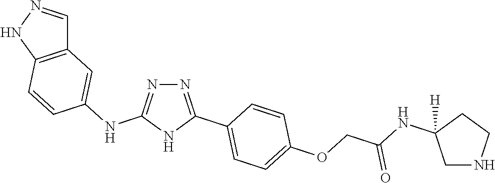 [SMILES: O=C(COc1ccc(-c2nnc(Nc3ccc4[nH]ncc4c3)[nH]2)cc1)N[C@H]1CCNC1] |
| 77 | 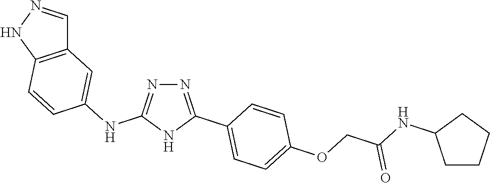 [SMILES: O=C(COc1ccc(-c2nnc(Nc3ccc4[nH]ncc4c3)[nH]2)cc1)NC1CCCC1] |
| 78 | 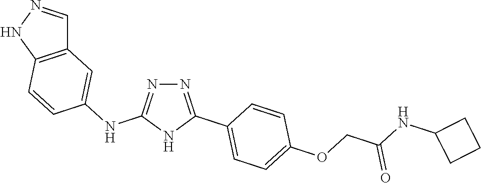 [SMILES: O=C(COc1ccc(-c2nnc(Nc3ccc4[nH]ncc4c3)[nH]2)cc1)NC1CCC1] |
| 79 | 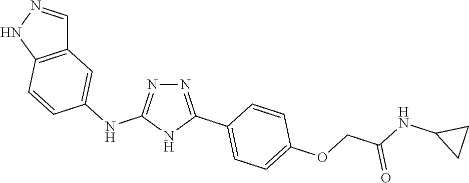 [SMILES: O=C(COc1ccc(-c2nnc(Nc3ccc4[nH]ncc4c3)[nH]2)cc1)NC1CC1] |
| 80 | 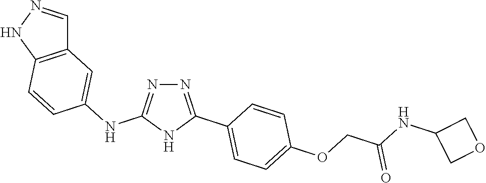 [SMILES: O=C(COc1ccc(-c2nnc(Nc3ccc4[nH]ncc4c3)[nH]2)cc1)NC1COC1] |
| 81 | 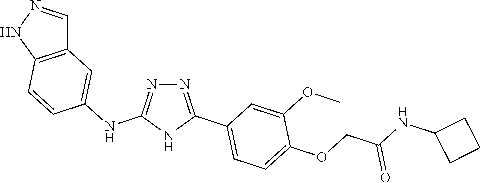 [SMILES: COc1cc(-c2nnc(Nc3ccc4[nH]ncc4c3)[nH]2)ccc1OCC(=O)NC1CCC1] |
| 82 | 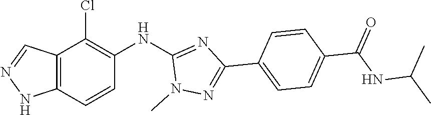 [SMILES: CC(C)NC(=O)c1ccc(-c2nc(Nc3ccc4[nH]ncc4c3Cl)n(C)n2)cc1] |
| 83 | 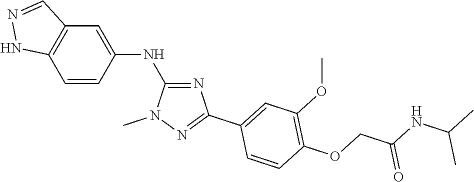 [SMILES: COc1cc(-c2nc(Nc3ccc4[nH]ncc4c3)n(C)n2)ccc1OCC(=O)NC(C)C] |
| 84 | 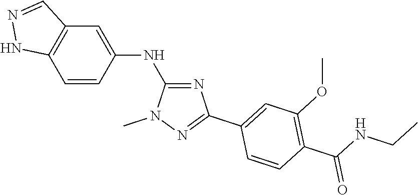 [SMILES: CCNC(=O)c1ccc(-c2nc(Nc3ccc4[nH]ncc4c3)n(C)n2)cc1OC] |
| 85 | 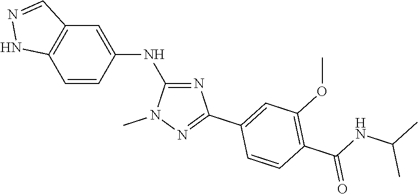 [SMILES: COc1cc(-c2nc(Nc3ccc4[nH]ncc4c3)n(C)n2)ccc1C(=O)NC(C)C] |
| 86 | 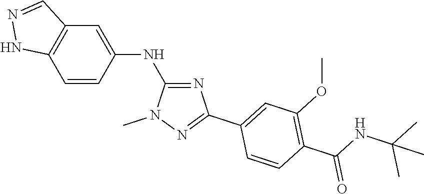 [SMILES: COc1cc(-c2nc(Nc3ccc4[nH]ncc4c3)n(C)n2)ccc1C(=O)NC(C)(C)C] |
| 87 | 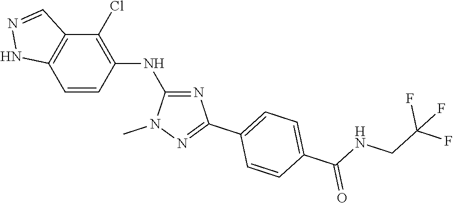 [SMILES: Cn1nc(-c2ccc(C(=O)NCC(F)(F)F)cc2)nc1Nc1ccc2[nH]ncc2c1Cl] |
| 88 | 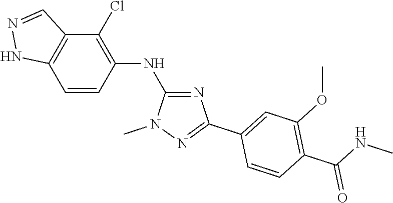 [SMILES: CNC(=O)c1ccc(-c2nc(Nc3ccc4[nH]ncc4c3Cl)n(C)n2)cc1OC] |
| 89 | 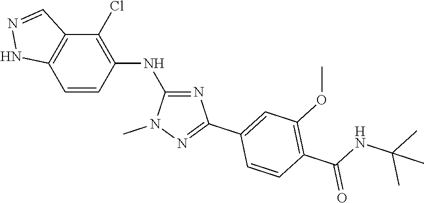 [SMILES: COc1cc(-c2nc(Nc3ccc4[nH]ncc4c3Cl)n(C)n2)ccc1C(=O)NC(C)(C)C] |
| 90 | 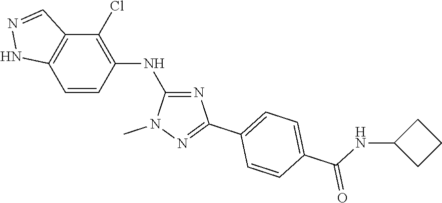 [SMILES: Cn1nc(-c2ccc(C(=O)NC3CCC3)cc2)nc1Nc1ccc2[nH]ncc2c1Cl] |
| 91 | 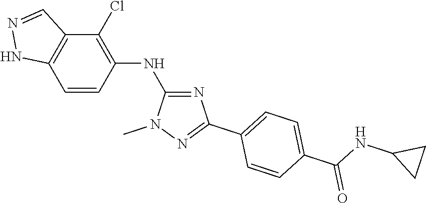 [SMILES: Cn1nc(-c2ccc(C(=O)NC3CC3)cc2)nc1Nc1ccc2[nH]ncc2c1Cl] |
| 92 | 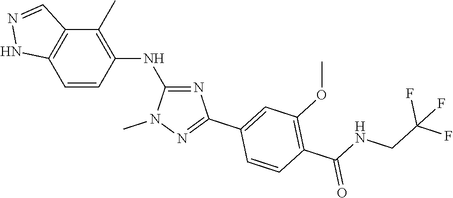 [SMILES: COc1cc(-c2nc(Nc3ccc4[nH]ncc4c3C)n(C)n2)ccc1C(=O)NCC(F)(F)F] |
| 93 | 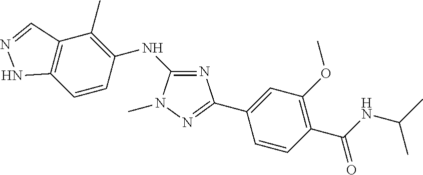 [SMILES: COc1cc(-c2nc(Nc3ccc4[nH]ncc4c3C)n(C)n2)ccc1C(=O)NC(C)C] |
| 94 | 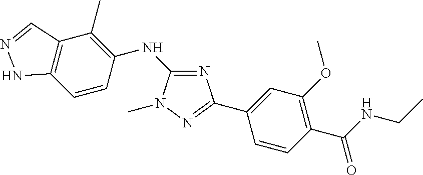 [SMILES: CCNC(=O)c1ccc(-c2nc(Nc3ccc4[nH]ncc4c3C)n(C)n2)cc1OC] |
| 95 | 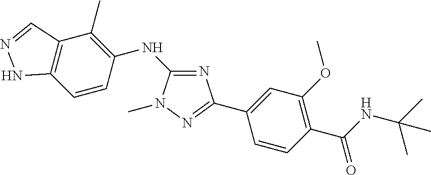 [SMILES: COc1cc(-c2nc(Nc3ccc4[nH]ncc4c3C)n(C)n2)ccc1C(=O)NC(C)(C)C] |
| 96 | 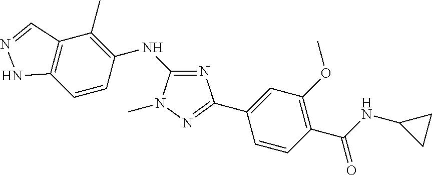 [SMILES: COc1cc(-c2nc(Nc3ccc4[nH]ncc4c3C)n(C)n2)ccc1C(=O)NC1CC1] |
| 97 | 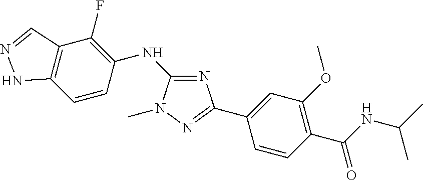 [SMILES: COc1cc(-c2nc(Nc3ccc4[nH]ncc4c3F)n(C)n2)ccc1C(=O)NC(C)C] |
| 98 | 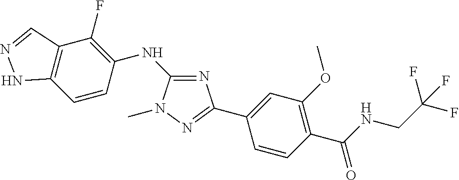 [SMILES: COc1cc(-c2nc(Nc3ccc4[nH]ncc4c3F)n(C)n2)ccc1C(=O)NCC(F)(F)F] |
| 99 | 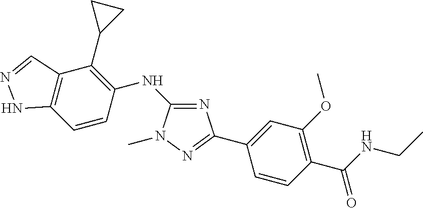 [SMILES: CCNC(=O)c1ccc(-c2nc(Nc3ccc4[nH]ncc4c3C3CC3)n(C)n2)cc1OC] |
| 100 | 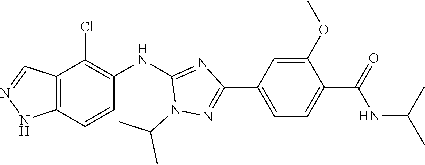 [SMILES: COc1cc(-c2nc(Nc3ccc4[nH]ncc4c3Cl)n(C(C)C)n2)ccc1C(=O)NC(C)C] |
| 101 | 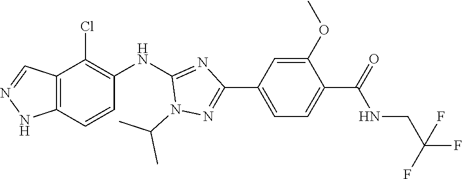 [SMILES: COc1cc(-c2nc(Nc3ccc4[nH]ncc4c3Cl)n(C(C)C)n2)ccc1C(=O)NCC(F)(F)F] |
| 102 | 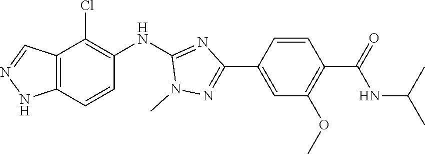 [SMILES: COc1cc(-c2nc(Nc3ccc4[nH]ncc4c3Cl)n(C)n2)ccc1C(=O)NC(C)C] |
| 103 | 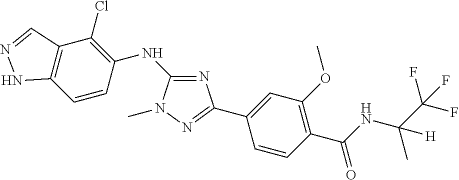 [SMILES: COc1cc(-c2nc(Nc3ccc4[nH]ncc4c3Cl)n(C)n2)ccc1C(=O)NC(C)C(F)(F)F] |
| 104 | 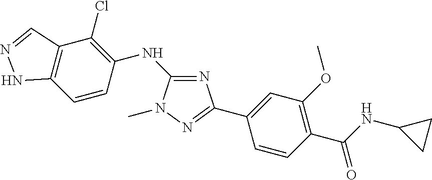 [SMILES: COc1cc(-c2nc(Nc3ccc4[nH]ncc4c3Cl)n(C)n2)ccc1C(=O)NC1CC1] |
| 105 | 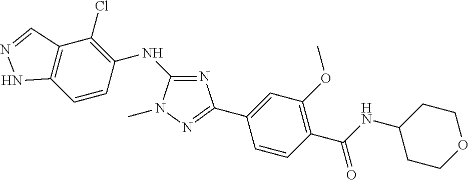 [SMILES: COc1cc(-c2nc(Nc3ccc4[nH]ncc4c3Cl)n(C)n2)ccc1C(=O)NC1CCOCC1] |
| 106 | 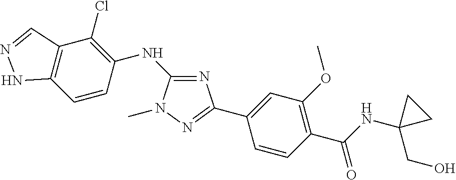 [SMILES: COc1cc(-c2nc(Nc3ccc4[nH]ncc4c3Cl)n(C)n2)ccc1C(=O)NC1(CO)CC1] |
| 107 | 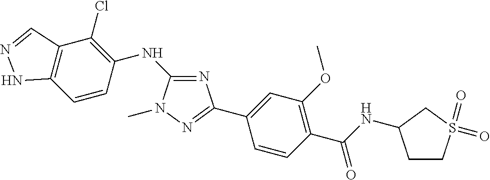 [SMILES: COc1cc(-c2nc(Nc3ccc4[nH]ncc4c3Cl)n(C)n2)ccc1C(=O)NC1CCS(=O)(=O)C1] |
| 108 | 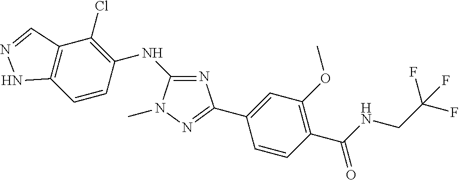 [SMILES: COc1cc(-c2nc(Nc3ccc4[nH]ncc4c3Cl)n(C)n2)ccc1C(=O)NCC(F)(F)F] |
| 109 | 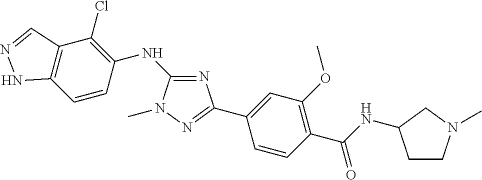 [SMILES: COc1cc(-c2nc(Nc3ccc4[nH]ncc4c3Cl)n(C)n2)ccc1C(=O)NC1CCN(C)C1] |
| 110 | 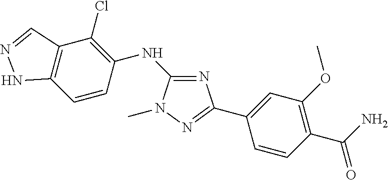 [SMILES: COc1cc(-c2nc(Nc3ccc4[nH]ncc4c3Cl)n(C)n2)ccc1C(N)=O] |
| 111 | 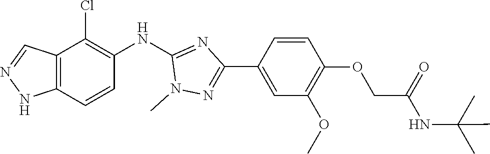 [SMILES: COc1cc(-c2nc(Nc3ccc4[nH]ncc4c3Cl)n(C)n2)ccc1OCC(=O)NC(C)(C)C] |
| 112 | 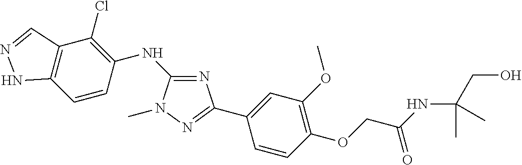 [SMILES: COc1cc(-c2nc(Nc3ccc4[nH]ncc4c3Cl)n(C)n2)ccc1OCC(=O)NC(C)(C)CO] |
| 113 | 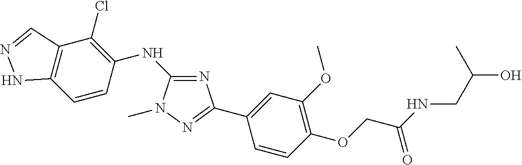 [SMILES: COc1cc(-c2nc(Nc3ccc4[nH]ncc4c3Cl)n(C)n2)ccc1OCC(=O)NCC(C)O] |
| 114 | 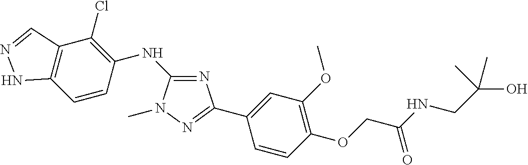 [SMILES: COc1cc(-c2nc(Nc3ccc4[nH]ncc4c3Cl)n(C)n2)ccc1OCC(=O)NCC(C)(C)O] |
| 115 | 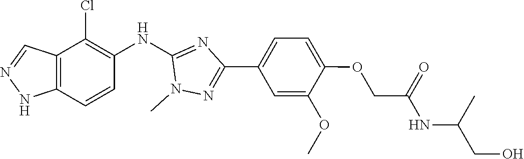 [SMILES: COc1cc(-c2nc(Nc3ccc4[nH]ncc4c3Cl)n(C)n2)ccc1OCC(=O)NC(C)CO] |
| 116 | 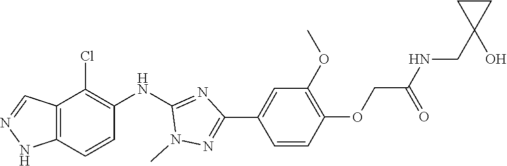 [SMILES: COc1cc(-c2nc(Nc3ccc4[nH]ncc4c3Cl)n(C)n2)ccc1OCC(=O)NCC1(O)CC1] |
| 117 | 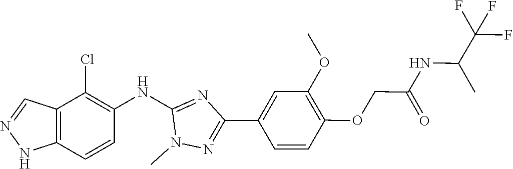 [SMILES: COc1cc(-c2nc(Nc3ccc4[nH]ncc4c3Cl)n(C)n2)ccc1OCC(=O)NC(C)C(F)(F)F] |
| 118 | 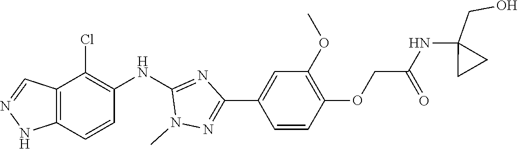 [SMILES: COc1cc(-c2nc(Nc3ccc4[nH]ncc4c3Cl)n(C)n2)ccc1OCC(=O)NC1(CO)CC1] |
| 119 | 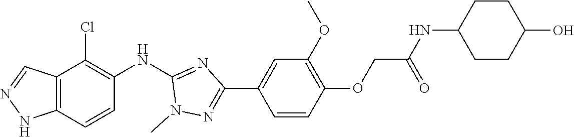 [SMILES: COc1cc(-c2nc(Nc3ccc4[nH]ncc4c3Cl)n(C)n2)ccc1OCC(=O)NC1CCC(O)CC1] |
| 120 | 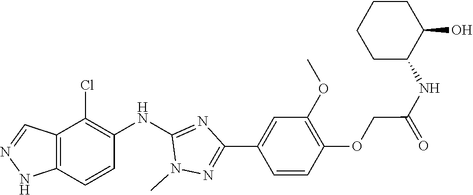 [SMILES: COc1cc(-c2nc(Nc3ccc4[nH]ncc4c3Cl)n(C)n2)ccc1OCC(=O)N[C@@H]1CCCC[C@H]1O] |
| 121 | 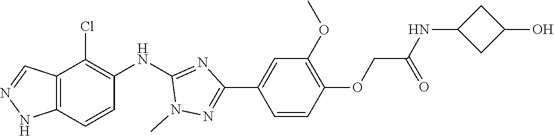 [SMILES: COc1cc(-c2nc(Nc3ccc4[nH]ncc4c3Cl)n(C)n2)ccc1OCC(=O)NC1CC(O)C1] |
| 122 | 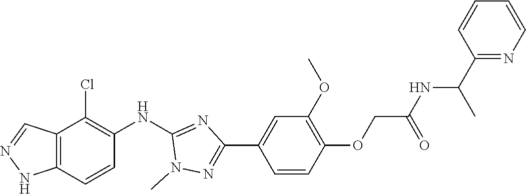 [SMILES: COc1cc(-c2nc(Nc3ccc4[nH]ncc4c3Cl)n(C)n2)ccc1OCC(=O)NC(C)c1ccccn1] |
| 123 | 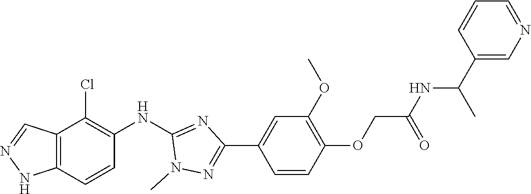 [SMILES: COc1cc(-c2nc(Nc3ccc4[nH]ncc4c3Cl)n(C)n2)ccc1OCC(=O)NC(C)c1cccnc1] |
| 124 | 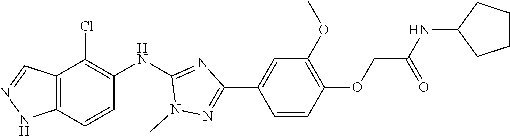 [SMILES: COc1cc(-c2nc(Nc3ccc4[nH]ncc4c3Cl)n(C)n2)ccc1OCC(=O)NC1CCCC1] |
| 125 | 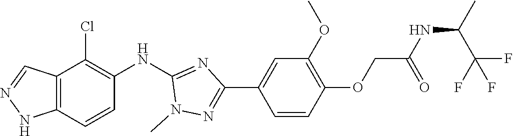 [SMILES: COc1cc(-c2nc(Nc3ccc4[nH]ncc4c3Cl)n(C)n2)ccc1OCC(=O)N[C@@H](C)C(F)(F)F] |
| 126 | 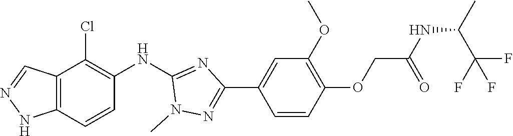 [SMILES: COc1cc(-c2nc(Nc3ccc4[nH]ncc4c3Cl)n(C)n2)ccc1OCC(=O)N[C@H](C)C(F)(F)F] |
| 127 | 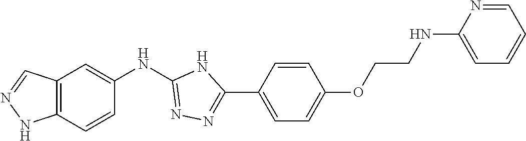 [SMILES: c1ccc(NCCOc2ccc(-c3nnc(Nc4ccc5[nH]ncc5c4)[nH]3)cc2)nc1] |
| 128 | 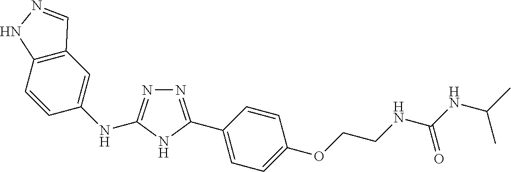 [SMILES: CC(C)NC(=O)NCCOc1ccc(-c2nnc(Nc3ccc4[nH]ncc4c3)[nH]2)cc1] |
| 129 | 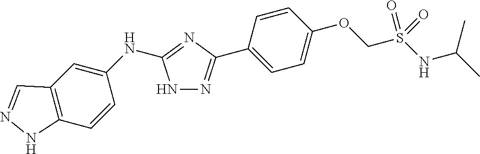 [SMILES: CC(C)NS(=O)(=O)COc1ccc(-c2n[nH]c(Nc3ccc4[nH]ncc4c3)n2)cc1] |
| 130 | 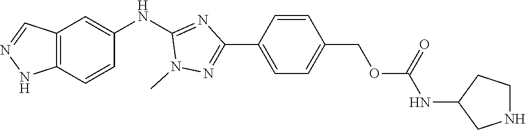 [SMILES: Cn1nc(-c2ccc(COC(=O)NC3CCNC3)cc2)nc1Nc1ccc2[nH]ncc2c1] |
| 131 | 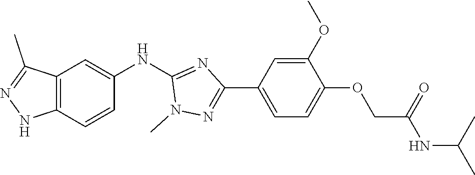 [SMILES: COc1cc(-c2nc(Nc3ccc4[nH]nc(C)c4c3)n(C)n2)ccc1OCC(=O)NC(C)C] |
| 132 | 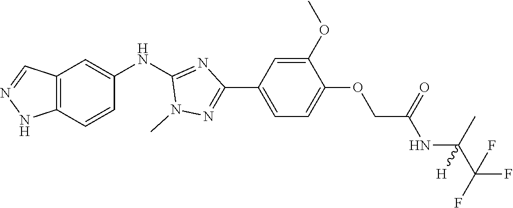 [SMILES: COc1cc(-c2nc(Nc3ccc4[nH]ncc4c3)n(C)n2)ccc1OCC(=O)NC(C)C(F)(F)F] |
| 133 | 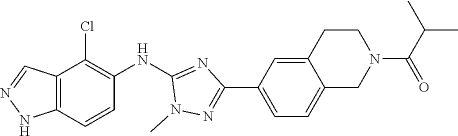 [SMILES: CC(C)C(=O)N1CCc2cc(-c3nc(Nc4ccc5[nH]ncc5c4Cl)n(C)n3)ccc2C1] |
| 134 | 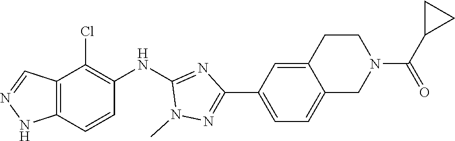 [SMILES: Cn1nc(-c2ccc3c(c2)CCN(C(=O)C2CC2)C3)nc1Nc1ccc2[nH]ncc2c1Cl] |
| 135 | 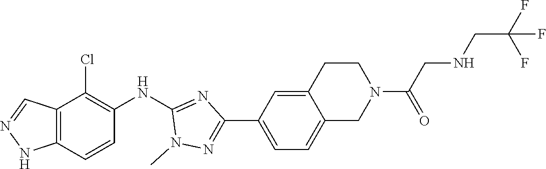 [SMILES: Cn1nc(-c2ccc3c(c2)CCN(C(=O)CNCC(F)(F)F)C3)nc1Nc1ccc2[nH]ncc2c1Cl] |
| 136 | 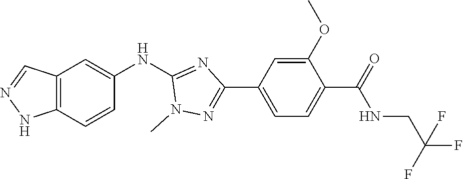 [SMILES: COc1cc(-c2nc(Nc3ccc4[nH]ncc4c3)n(C)n2)ccc1C(=O)NCC(F)(F)F] |
| 137 | 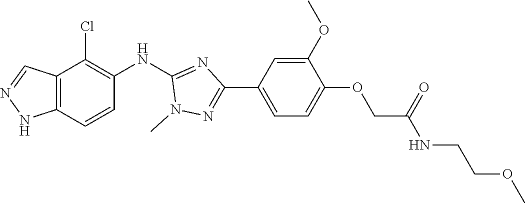 [SMILES: COCCNC(=O)COc1ccc(-c2nc(Nc3ccc4[nH]ncc4c3Cl)n(C)n2)cc1OC] |
| 138 | 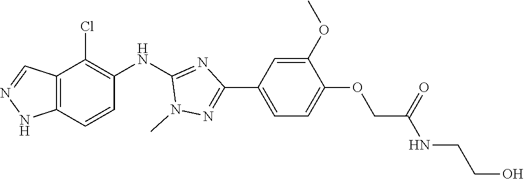 [SMILES: COc1cc(-c2nc(Nc3ccc4[nH]ncc4c3Cl)n(C)n2)ccc1OCC(=O)NCCO] |
| 139 | 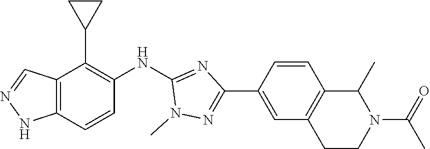 [SMILES: CC(=O)N1CCc2cc(-c3nc(Nc4ccc5[nH]ncc5c4C4CC4)n(C)n3)ccc2C1C] |
| 140 | 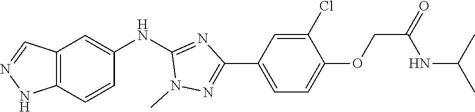 [SMILES: CC(C)NC(=O)COc1ccc(-c2nc(Nc3ccc4[nH]ncc4c3)n(C)n2)cc1Cl] |
| 141 | 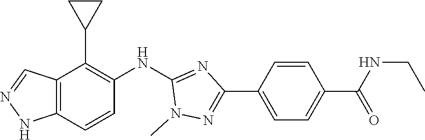 [SMILES: CCNC(=O)c1ccc(-c2nc(Nc3ccc4[nH]ncc4c3C3CC3)n(C)n2)cc1] |
| 142 | 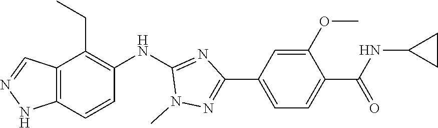 [SMILES: CCc1c(Nc2nc(-c3ccc(C(=O)NC4CC4)c(OC)c3)nn2C)ccc2[nH]ncc12] |
| 143 | 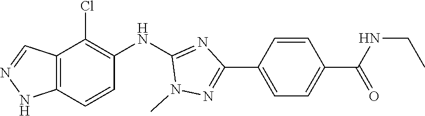 [SMILES: CCNC(=O)c1ccc(-c2nc(Nc3ccc4[nH]ncc4c3Cl)n(C)n2)cc1] |
| 144 | 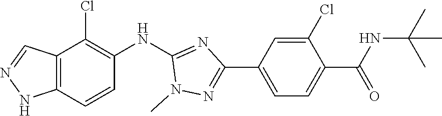 [SMILES: Cn1nc(-c2ccc(C(=O)NC(C)(C)C)c(Cl)c2)nc1Nc1ccc2[nH]ncc2c1Cl] |
| 145 | 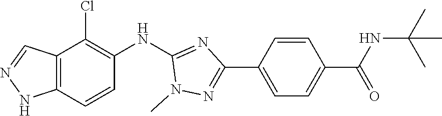 [SMILES: Cn1nc(-c2ccc(C(=O)NC(C)(C)C)cc2)nc1Nc1ccc2[nH]ncc2c1Cl] |
| 146 | 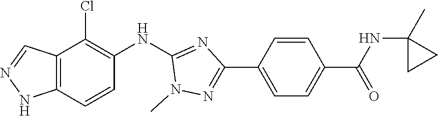 [SMILES: Cn1nc(-c2ccc(C(=O)NC3(C)CC3)cc2)nc1Nc1ccc2[nH]ncc2c1Cl] |
| 147 | 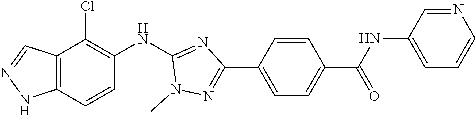 [SMILES: Cn1nc(-c2ccc(C(=O)Nc3cccnc3)cc2)nc1Nc1ccc2[nH]ncc2c1Cl] |
| 148 | 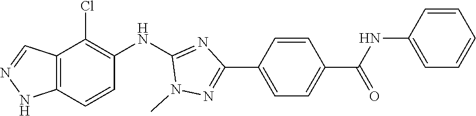 [SMILES: Cn1nc(-c2ccc(C(=O)Nc3ccccc3)cc2)nc1Nc1ccc2[nH]ncc2c1Cl] |
| 149 | 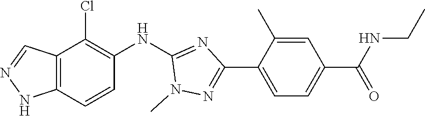 [SMILES: CCNC(=O)c1ccc(-c2nc(Nc3ccc4[nH]ncc4c3Cl)n(C)n2)c(C)c1] |
| 150 | 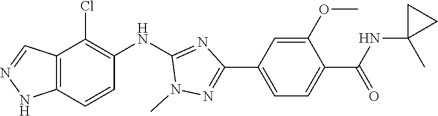 [SMILES: COc1cc(-c2nc(Nc3ccc4[nH]ncc4c3Cl)n(C)n2)ccc1C(=O)NC1(C)CC1] |
| 151 | 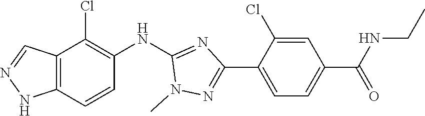 [SMILES: CCNC(=O)c1ccc(-c2nc(Nc3ccc4[nH]ncc4c3Cl)n(C)n2)c(Cl)c1] |
| 152 | 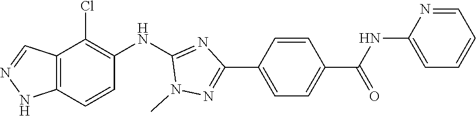 [SMILES: Cn1nc(-c2ccc(C(=O)Nc3ccccn3)cc2)nc1Nc1ccc2[nH]ncc2c1Cl] |
| 153 | 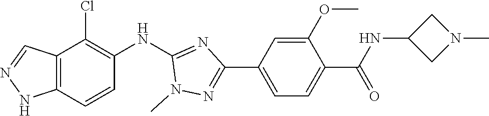 [SMILES: COc1cc(-c2nc(Nc3ccc4[nH]ncc4c3Cl)n(C)n2)ccc1C(=O)NC1CN(C)C1] |
| 154 | 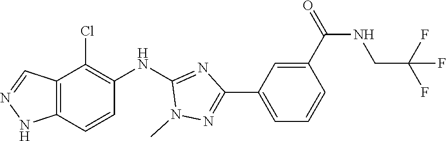 [SMILES: Cn1nc(-c2cccc(C(=O)NCC(F)(F)F)c2)nc1Nc1ccc2[nH]ncc2c1Cl] |
| 155 | 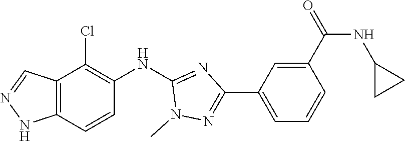 [SMILES: Cn1nc(-c2cccc(C(=O)NC3CC3)c2)nc1Nc1ccc2[nH]ncc2c1Cl] |
| 156 | 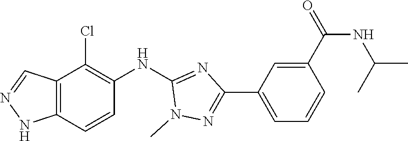 [SMILES: CC(C)NC(=O)c1cccc(-c2nc(Nc3ccc4[nH]ncc4c3Cl)n(C)n2)c1] |
| 157 | 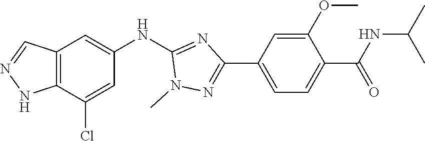 [SMILES: COc1cc(-c2nc(Nc3cc(Cl)c4[nH]ncc4c3)n(C)n2)ccc1C(=O)NC(C)C] |
| 158 | 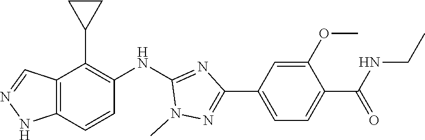 [SMILES: CCNC(=O)c1ccc(-c2nc(Nc3ccc4[nH]ncc4c3C3CC3)n(C)n2)cc1OC] |
| 159 | 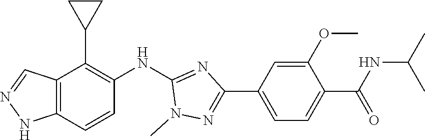 [SMILES: COc1cc(-c2nc(Nc3ccc4[nH]ncc4c3C3CC3)n(C)n2)ccc1C(=O)NC(C)C] |
| 160 | 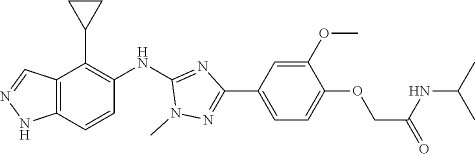 [SMILES: COc1cc(-c2nc(Nc3ccc4[nH]ncc4c3C3CC3)n(C)n2)ccc1OCC(=O)NC(C)C] |
| 161 | 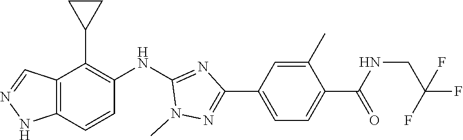 [SMILES: Cc1cc(-c2nc(Nc3ccc4[nH]ncc4c3C3CC3)n(C)n2)ccc1C(=O)NCC(F)(F)F] |
| 162 | 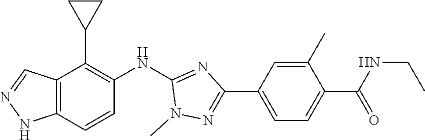 [SMILES: CCNC(=O)c1ccc(-c2nc(Nc3ccc4[nH]ncc4c3C3CC3)n(C)n2)cc1C] |
| 163 | 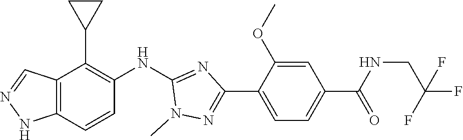 [SMILES: COc1cc(C(=O)NCC(F)(F)F)ccc1-c1nc(Nc2ccc3[nH]ncc3c2C2CC2)n(C)n1] |
| 164 | 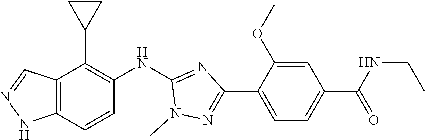 [SMILES: CCNC(=O)c1ccc(-c2nc(Nc3ccc4[nH]ncc4c3C3CC3)n(C)n2)c(OC)c1] |
| 165 | 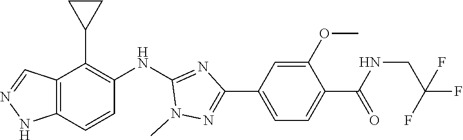 [SMILES: COc1cc(-c2nc(Nc3ccc4[nH]ncc4c3C3CC3)n(C)n2)ccc1C(=O)NCC(F)(F)F] |
| 166 | 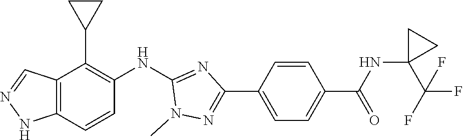 [SMILES: Cn1nc(-c2ccc(C(=O)NC3(C(F)(F)F)CC3)cc2)nc1Nc1ccc2[nH]ncc2c1C1CC1] |
| 167 | 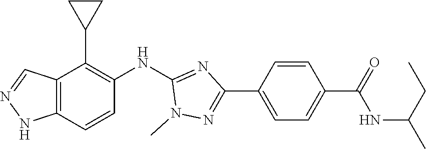 [SMILES: CCC(C)NC(=O)c1ccc(-c2nc(Nc3ccc4[nH]ncc4c3C3CC3)n(C)n2)cc1] |
| 168 | 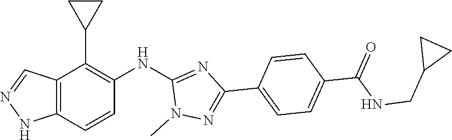 [SMILES: Cn1nc(-c2ccc(C(=O)NCC3CC3)cc2)nc1Nc1ccc2[nH]ncc2c1C1CC1] |
| 169 | 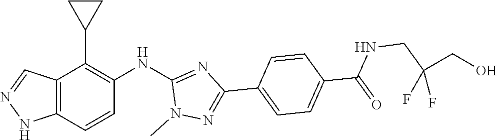 [SMILES: Cn1nc(-c2ccc(C(=O)NCC(F)(F)CO)cc2)nc1Nc1ccc2[nH]ncc2c1C1CC1] |
| 170 | 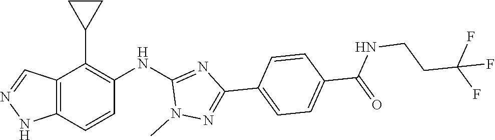 [SMILES: Cn1nc(-c2ccc(C(=O)NCCC(F)(F)F)cc2)nc1Nc1ccc2[nH]ncc2c1C1CC1] |
| 171 | 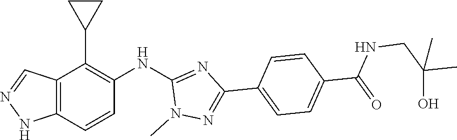 [SMILES: Cn1nc(-c2ccc(C(=O)NCC(C)(C)O)cc2)nc1Nc1ccc2[nH]ncc2c1C1CC1] |
| 172 | 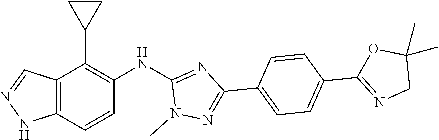 [SMILES: Cn1nc(-c2ccc(C3=NCC(C)(C)O3)cc2)nc1Nc1ccc2[nH]ncc2c1C1CC1] |
| 173 | 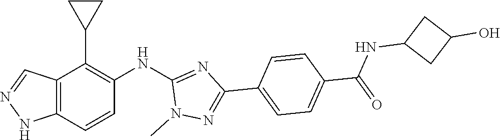 [SMILES: Cn1nc(-c2ccc(C(=O)NC3CC(O)C3)cc2)nc1Nc1ccc2[nH]ncc2c1C1CC1] |
| 174 | 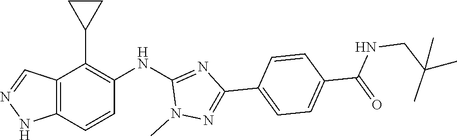 [SMILES: Cn1nc(-c2ccc(C(=O)NCC(C)(C)C)cc2)nc1Nc1ccc2[nH]ncc2c1C1CC1] |
| 175 | 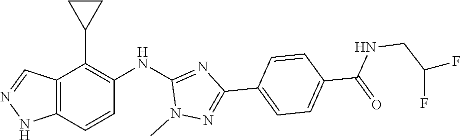 [SMILES: Cn1nc(-c2ccc(C(=O)NCC(F)F)cc2)nc1Nc1ccc2[nH]ncc2c1C1CC1] |
| 176 | 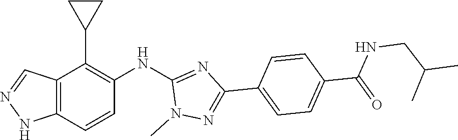 [SMILES: CC(C)CNC(=O)c1ccc(-c2nc(Nc3ccc4[nH]ncc4c3C3CC3)n(C)n2)cc1] |
| 177 | 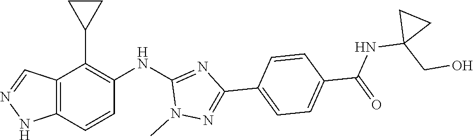 [SMILES: Cn1nc(-c2ccc(C(=O)NC3(CO)CC3)cc2)nc1Nc1ccc2[nH]ncc2c1C1CC1] |
| 178 | 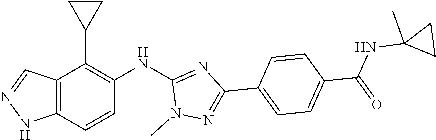 [SMILES: Cn1nc(-c2ccc(C(=O)NC3(C)CC3)cc2)nc1Nc1ccc2[nH]ncc2c1C1CC1] |
| 179 | 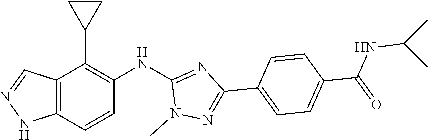 [SMILES: CC(C)NC(=O)c1ccc(-c2nc(Nc3ccc4[nH]ncc4c3C3CC3)n(C)n2)cc1] |
| 180 | 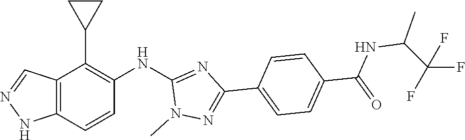 [SMILES: CC(NC(=O)c1ccc(-c2nc(Nc3ccc4[nH]ncc4c3C3CC3)n(C)n2)cc1)C(F)(F)F] |
| 181 | 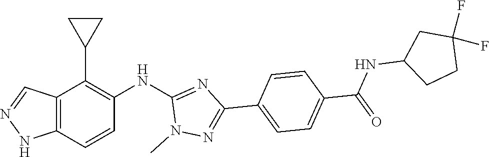 [SMILES: Cn1nc(-c2ccc(C(=O)NC3CCC(F)(F)C3)cc2)nc1Nc1ccc2[nH]ncc2c1C1CC1] |
| 182 | 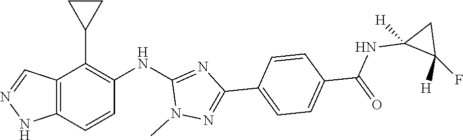 [SMILES: Cn1nc(-c2ccc(C(=O)N[C@H]3C[C@@H]3F)cc2)nc1Nc1ccc2[nH]ncc2c1C1CC1] |
| 183 | 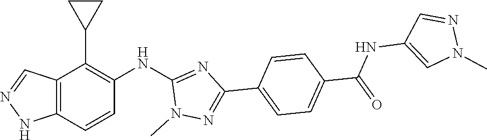 [SMILES: Cn1cc(NC(=O)c2ccc(-c3nc(Nc4ccc5[nH]ncc5c4C4CC4)n(C)n3)cc2)cn1] |
| 184 | 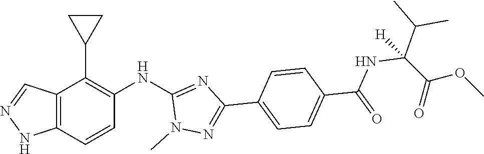 [SMILES: COC(=O)[C@@H](NC(=O)c1ccc(-c2nc(Nc3ccc4[nH]ncc4c3C3CC3)n(C)n2)cc1)C(C)C] |
| 185 | 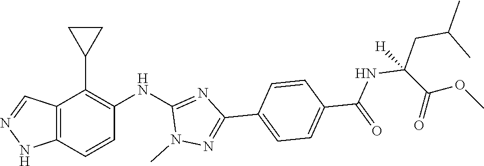 [SMILES: COC(=O)[C@H](CC(C)C)NC(=O)c1ccc(-c2nc(Nc3ccc4[nH]ncc4c3C3CC3)n(C)n2)cc1] |
| 186 | 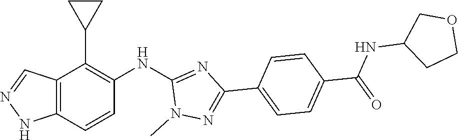 [SMILES: Cn1nc(-c2ccc(C(=O)NC3CCOC3)cc2)nc1Nc1ccc2[nH]ncc2c1C1CC1] |
| 187 | 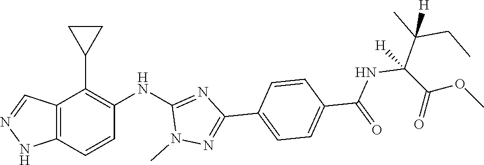 [SMILES: CC[C@@H](C)[C@H](NC(=O)c1ccc(-c2nc(Nc3ccc4[nH]ncc4c3C3CC3)n(C)n2)cc1)C(=O)OC] |
| 188 | 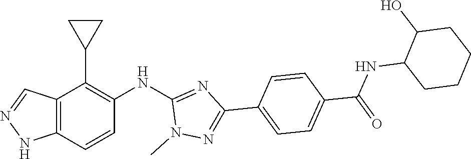 [SMILES: Cn1nc(-c2ccc(C(=O)NC3CCCCC3O)cc2)nc1Nc1ccc2[nH]ncc2c1C1CC1] |
| 189 | 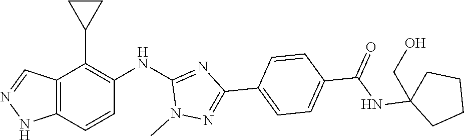 [SMILES: Cn1nc(-c2ccc(C(=O)NC3(CO)CCCC3)cc2)nc1Nc1ccc2[nH]ncc2c1C1CC1] |
| 190 | 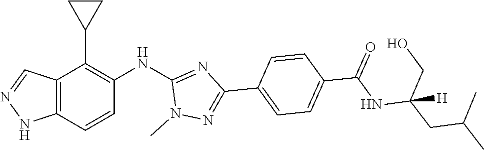 [SMILES: CC(C)C[C@@H](CO)NC(=O)c1ccc(-c2nc(Nc3ccc4[nH]ncc4c3C3CC3)n(C)n2)cc1] |
| 191 | 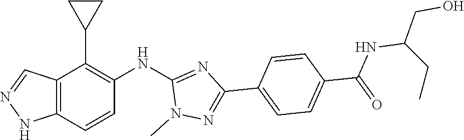 [SMILES: CCC(CO)NC(=O)c1ccc(-c2nc(Nc3ccc4[nH]ncc4c3C3CC3)n(C)n2)cc1] |
| 192 | 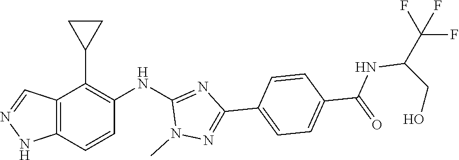 [SMILES: Cn1nc(-c2ccc(C(=O)NC(CO)C(F)(F)F)cc2)nc1Nc1ccc2[nH]ncc2c1C1CC1] |
| 193 | 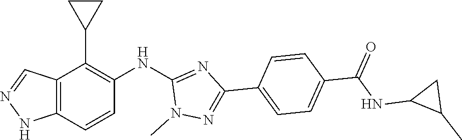 [SMILES: CC1CC1NC(=O)c1ccc(-c2nc(Nc3ccc4[nH]ncc4c3C3CC3)n(C)n2)cc1] |
| 194 | 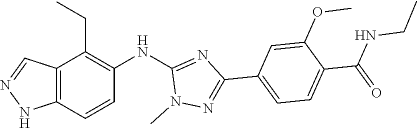 [SMILES: CCNC(=O)c1ccc(-c2nc(Nc3ccc4[nH]ncc4c3CC)n(C)n2)cc1OC] |
| 195 | 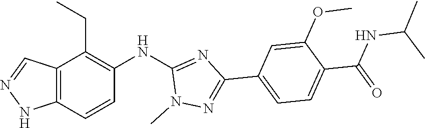 [SMILES: CCc1c(Nc2nc(-c3ccc(C(=O)NC(C)C)c(OC)c3)nn2C)ccc2[nH]ncc12] |
| 196 | 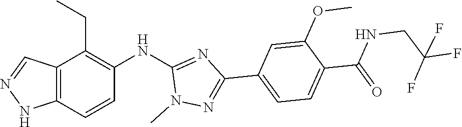 [SMILES: CCc1c(Nc2nc(-c3ccc(C(=O)NCC(F)(F)F)c(OC)c3)nn2C)ccc2[nH]ncc12] |
| 197 | 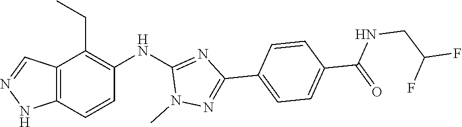 [SMILES: CCc1c(Nc2nc(-c3ccc(C(=O)NCC(F)F)cc3)nn2C)ccc2[nH]ncc12] |
| 198 | 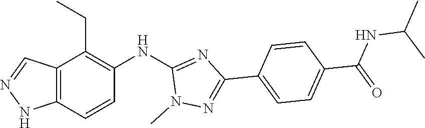 [SMILES: CCc1c(Nc2nc(-c3ccc(C(=O)NC(C)C)cc3)nn2C)ccc2[nH]ncc12] |
| 199 | 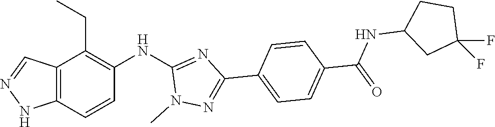 [SMILES: CCc1c(Nc2nc(-c3ccc(C(=O)NC4CCC(F)(F)C4)cc3)nn2C)ccc2[nH]ncc12] |
| 200 | 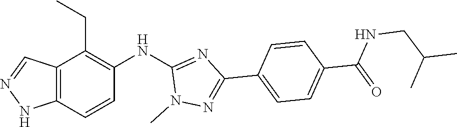 [SMILES: CCc1c(Nc2nc(-c3ccc(C(=O)NCC(C)C)cc3)nn2C)ccc2[nH]ncc12] |
| 201 | 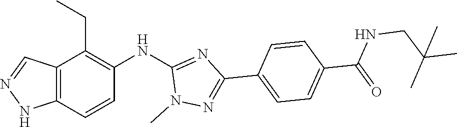 [SMILES: CCc1c(Nc2nc(-c3ccc(C(=O)NCC(C)(C)C)cc3)nn2C)ccc2[nH]ncc12] |
| 202 | 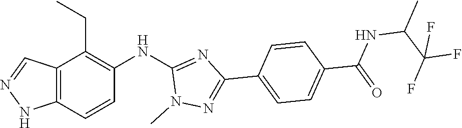 [SMILES: CCc1c(Nc2nc(-c3ccc(C(=O)NC(C)C(F)(F)F)cc3)nn2C)ccc2[nH]ncc12] |
| 203 | 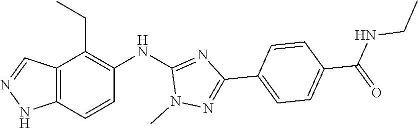 [SMILES: CCNC(=O)c1ccc(-c2nc(Nc3ccc4[nH]ncc4c3CC)n(C)n2)cc1] |
| 204 | 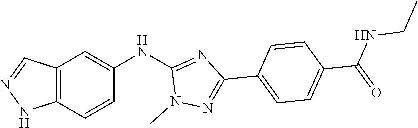 [SMILES: CCNC(=O)c1ccc(-c2nc(Nc3ccc4[nH]ncc4c3)n(C)n2)cc1] |
| 205 | 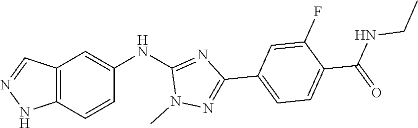 [SMILES: CCNC(=O)c1ccc(-c2nc(Nc3ccc4[nH]ncc4c3)n(C)n2)cc1F] |
| 206 | 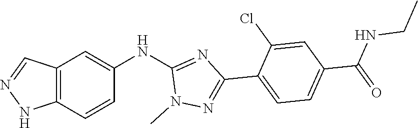 [SMILES: CCNC(=O)c1ccc(-c2nc(Nc3ccc4[nH]ncc4c3)n(C)n2)c(Cl)c1] |
| 207 | 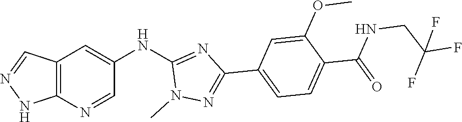 [SMILES: COc1cc(-c2nc(Nc3cnc4[nH]ncc4c3)n(C)n2)ccc1C(=O)NCC(F)(F)F] |
| 208 | 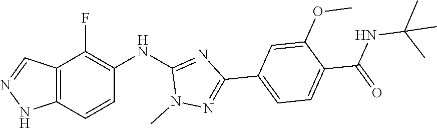 [SMILES: COc1cc(-c2nc(Nc3ccc4[nH]ncc4c3F)n(C)n2)ccc1C(=O)NC(C)(C)C] |
| 209 | 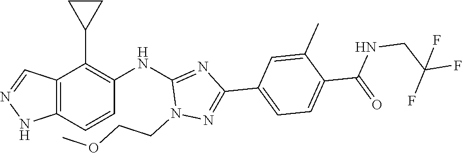 [SMILES: COCCn1nc(-c2ccc(C(=O)NCC(F)(F)F)c(C)c2)nc1Nc1ccc2[nH]ncc2c1C1CC1] |
| 210 | 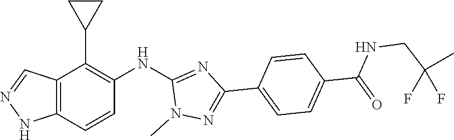 [SMILES: Cn1nc(-c2ccc(C(=O)NCC(C)(F)F)cc2)nc1Nc1ccc2[nH]ncc2c1C1CC1] |
| 211 | 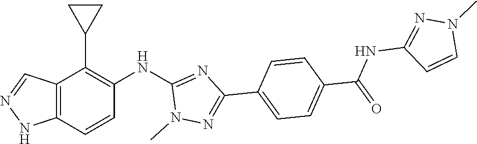 [SMILES: Cn1ccc(NC(=O)c2ccc(-c3nc(Nc4ccc5[nH]ncc5c4C4CC4)n(C)n3)cc2)n1] |
| 212 | 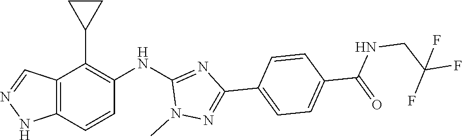 [SMILES: Cn1nc(-c2ccc(C(=O)NCC(F)(F)F)cc2)nc1Nc1ccc2[nH]ncc2c1C1CC1] |
| 213 | 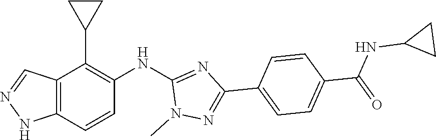 [SMILES: Cn1nc(-c2ccc(C(=O)NC3CC3)cc2)nc1Nc1ccc2[nH]ncc2c1C1CC1] |
| 214 | 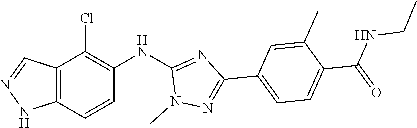 [SMILES: CCNC(=O)c1ccc(-c2nc(Nc3ccc4[nH]ncc4c3Cl)n(C)n2)cc1C] |
| 215 | 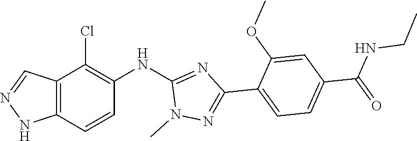 [SMILES: CCNC(=O)c1ccc(-c2nc(Nc3ccc4[nH]ncc4c3Cl)n(C)n2)c(OC)c1] |
| 216 | 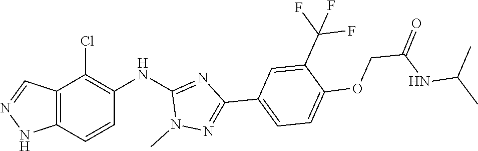 [SMILES: CC(C)NC(=O)COc1ccc(-c2nc(Nc3ccc4[nH]ncc4c3Cl)n(C)n2)cc1C(F)(F)F] |
| 217 | 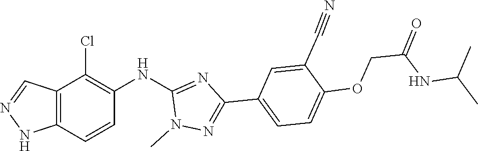 [SMILES: CC(C)NC(=O)COc1ccc(-c2nc(Nc3ccc4[nH]ncc4c3Cl)n(C)n2)cc1C#N] |
| 218 | 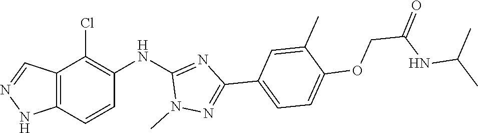 [SMILES: Cc1cc(-c2nc(Nc3ccc4[nH]ncc4c3Cl)n(C)n2)ccc1OCC(=O)NC(C)C] |
| 219 | 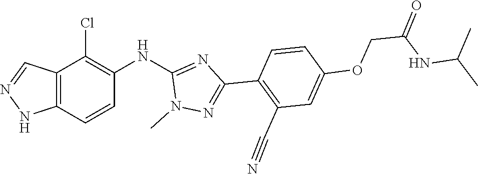 [SMILES: CC(C)NC(=O)COc1ccc(-c2nc(Nc3ccc4[nH]ncc4c3Cl)n(C)n2)c(C#N)c1] |
| 220 | 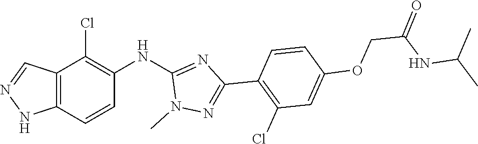 [SMILES: CC(C)NC(=O)COc1ccc(-c2nc(Nc3ccc4[nH]ncc4c3Cl)n(C)n2)c(Cl)c1] |
| 221 | 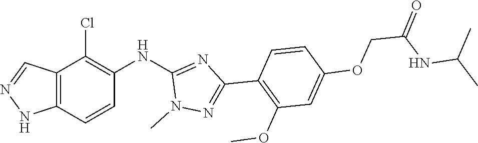 [SMILES: COc1cc(OCC(=O)NC(C)C)ccc1-c1nc(Nc2ccc3[nH]ncc3c2Cl)n(C)n1] |
| 222 | 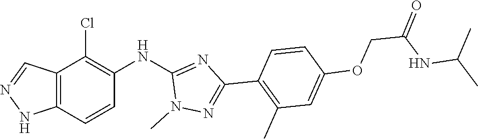 [SMILES: Cc1cc(OCC(=O)NC(C)C)ccc1-c1nc(Nc2ccc3[nH]ncc3c2Cl)n(C)n1] |
| 223 | 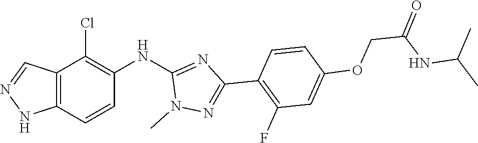 [SMILES: CC(C)NC(=O)COc1ccc(-c2nc(Nc3ccc4[nH]ncc4c3Cl)n(C)n2)c(F)c1] |
| 224 | 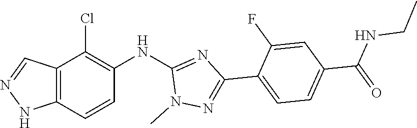 [SMILES: CCNC(=O)c1ccc(-c2nc(Nc3ccc4[nH]ncc4c3Cl)n(C)n2)c(F)c1] |
| 225 | 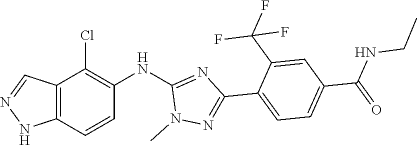 [SMILES: CCNC(=O)c1ccc(-c2nc(Nc3ccc4[nH]ncc4c3Cl)n(C)n2)c(C(F)(F)F)c1] |
| 226 | 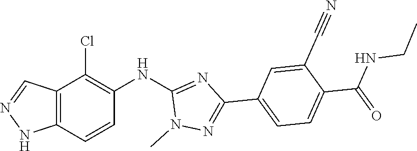 [SMILES: CCNC(=O)c1ccc(-c2nc(Nc3ccc4[nH]ncc4c3Cl)n(C)n2)cc1C#N] |
| 227 | 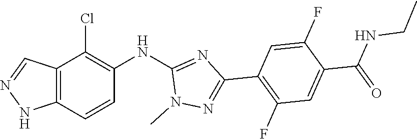 [SMILES: CCNC(=O)c1cc(F)c(-c2nc(Nc3ccc4[nH]ncc4c3Cl)n(C)n2)cc1F] |
| 228 | 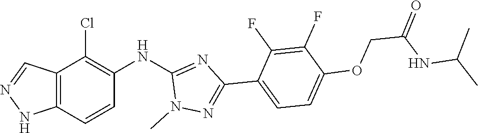 [SMILES: CC(C)NC(=O)COc1ccc(-c2nc(Nc3ccc4[nH]ncc4c3Cl)n(C)n2)c(F)c1F] |
| 229 | 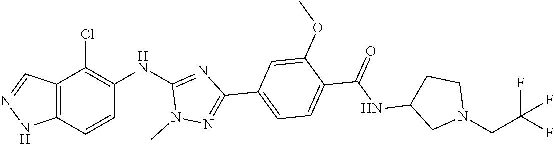 [SMILES: COc1cc(-c2nc(Nc3ccc4[nH]ncc4c3Cl)n(C)n2)ccc1C(=O)NC1CCN(CC(F)(F)F)C1] |
| 230 | 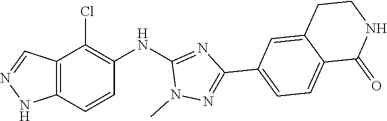 [SMILES: Cn1nc(-c2ccc3c(c2)CCNC3=O)nc1Nc1ccc2[nH]ncc2c1Cl] |
| 231 | 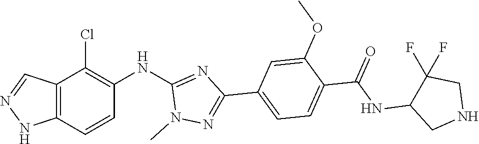 [SMILES: COc1cc(-c2nc(Nc3ccc4[nH]ncc4c3Cl)n(C)n2)ccc1C(=O)NC1CNCC1(F)F] |
| 232 | 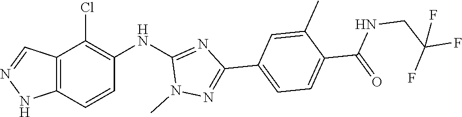 [SMILES: Cc1cc(-c2nc(Nc3ccc4[nH]ncc4c3Cl)n(C)n2)ccc1C(=O)NCC(F)(F)F] |
| 233 | 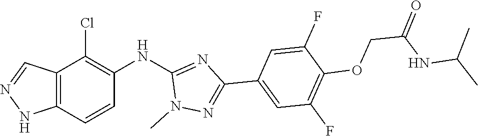 [SMILES: CC(C)NC(=O)COc1c(F)cc(-c2nc(Nc3ccc4[nH]ncc4c3Cl)n(C)n2)cc1F] |
| 234 | 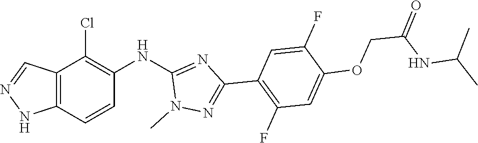 [SMILES: CC(C)NC(=O)COc1cc(F)c(-c2nc(Nc3ccc4[nH]ncc4c3Cl)n(C)n2)cc1F] |
| 235 | 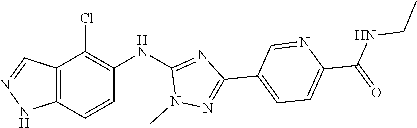 [SMILES: CCNC(=O)c1ccc(-c2nc(Nc3ccc4[nH]ncc4c3Cl)n(C)n2)cn1] |
| 236 | 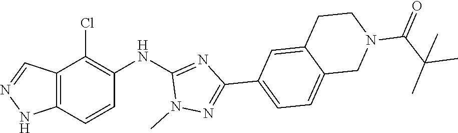 [SMILES: Cn1nc(-c2ccc3c(c2)CCN(C(=O)C(C)(C)C)C3)nc1Nc1ccc2[nH]ncc2c1Cl] |
| 237 | 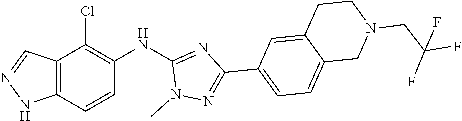 [SMILES: Cn1nc(-c2ccc3c(c2)CCN(CC(F)(F)F)C3)nc1Nc1ccc2[nH]ncc2c1Cl] |
| 238 | 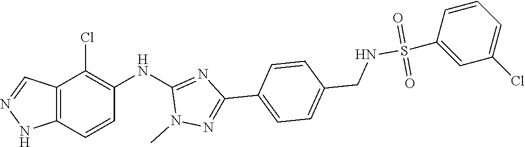 [SMILES: Cn1nc(-c2ccc(CNS(=O)(=O)c3cccc(Cl)c3)cc2)nc1Nc1ccc2[nH]ncc2c1Cl] |
| 239 | 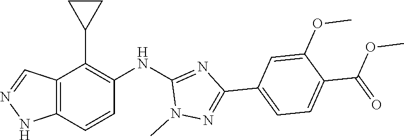 [SMILES: COC(=O)c1ccc(-c2nc(Nc3ccc4[nH]ncc4c3C3CC3)n(C)n2)cc1OC] |
| 240 | 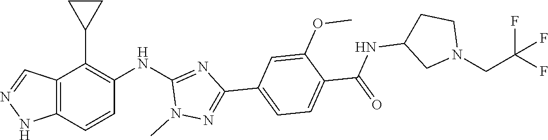 [SMILES: COc1cc(-c2nc(Nc3ccc4[nH]ncc4c3C3CC3)n(C)n2)ccc1C(=O)NC1CCN(CC(F)(F)F)C1] |
| 241 | 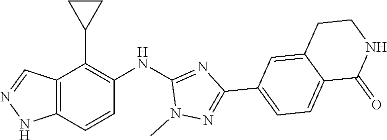 [SMILES: Cn1nc(-c2ccc3c(c2)CCNC3=O)nc1Nc1ccc2[nH]ncc2c1C1CC1] |
| 242 | 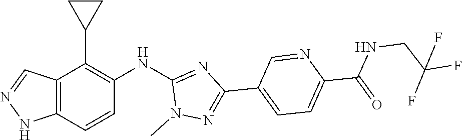 [SMILES: Cn1nc(-c2ccc(C(=O)NCC(F)(F)F)nc2)nc1Nc1ccc2[nH]ncc2c1C1CC1] |
| 243 | 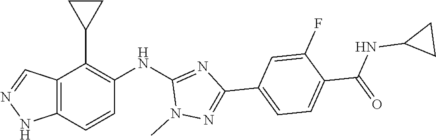 [SMILES: Cn1nc(-c2ccc(C(=O)NC3CC3)c(F)c2)nc1Nc1ccc2[nH]ncc2c1C1CC1] |
| 244 | 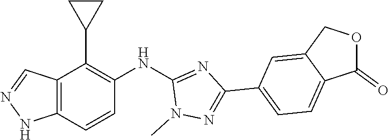 [SMILES: Cn1nc(-c2ccc3c(c2)COC3=O)nc1Nc1ccc2[nH]ncc2c1C1CC1] |
| 245 | 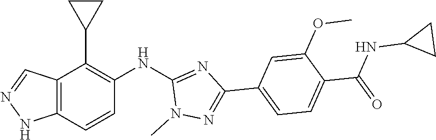 [SMILES: COc1cc(-c2nc(Nc3ccc4[nH]ncc4c3C3CC3)n(C)n2)ccc1C(=O)NC1CC1] |
| 246 | 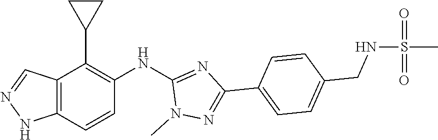 [SMILES: Cn1nc(-c2ccc(CNS(C)(=O)=O)cc2)nc1Nc1ccc2[nH]ncc2c1C1CC1] |
| 247 | 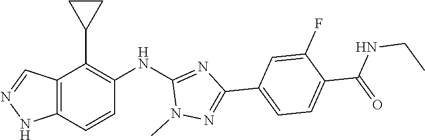 [SMILES: CCNC(=O)c1ccc(-c2nc(Nc3ccc4[nH]ncc4c3C3CC3)n(C)n2)cc1F] |
| 248 | 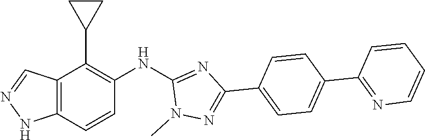 [SMILES: Cn1nc(-c2ccc(-c3ccccn3)cc2)nc1Nc1ccc2[nH]ncc2c1C1CC1] |
| 249 | 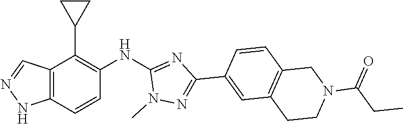 [SMILES: CCC(=O)N1CCc2cc(-c3nc(Nc4ccc5[nH]ncc5c4C4CC4)n(C)n3)ccc2C1] |
| 250 | 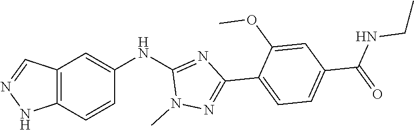 [SMILES: CCNC(=O)c1ccc(-c2nc(Nc3ccc4[nH]ncc4c3)n(C)n2)c(OC)c1] |
| 251 | 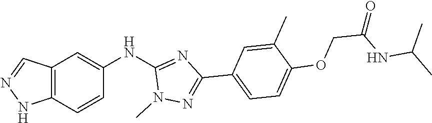 [SMILES: Cc1cc(-c2nc(Nc3ccc4[nH]ncc4c3)n(C)n2)ccc1OCC(=O)NC(C)C] |
| 252 | 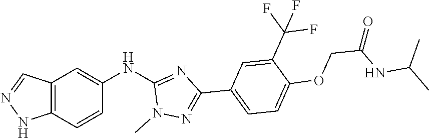 [SMILES: CC(C)NC(=O)COc1ccc(-c2nc(Nc3ccc4[nH]ncc4c3)n(C)n2)cc1C(F)(F)F] |
| 253 | 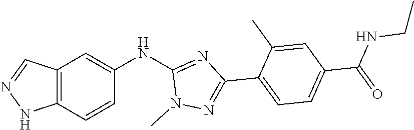 [SMILES: CCNC(=O)c1ccc(-c2nc(Nc3ccc4[nH]ncc4c3)n(C)n2)c(C)c1] |
| 254 | 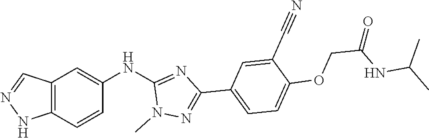 [SMILES: CC(C)NC(=O)COc1ccc(-c2nc(Nc3ccc4[nH]ncc4c3)n(C)n2)cc1C#N] |
| 255 | 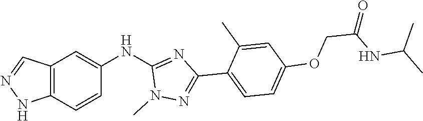 [SMILES: Cc1cc(OCC(=O)NC(C)C)ccc1-c1nc(Nc2ccc3[nH]ncc3c2)n(C)n1] |
| 256 | 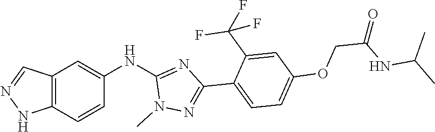 [SMILES: CC(C)NC(=O)COc1ccc(-c2nc(Nc3ccc4[nH]ncc4c3)n(C)n2)c(C(F)(F)F)c1] |
| 257 | 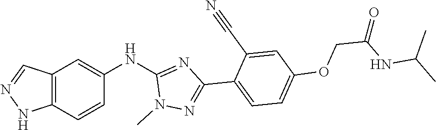 [SMILES: CC(C)NC(=O)COc1ccc(-c2nc(Nc3ccc4[nH]ncc4c3)n(C)n2)c(C#N)c1] |
| 258 | 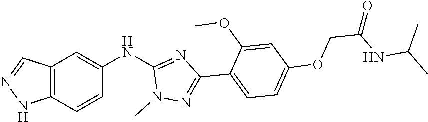 [SMILES: COc1cc(OCC(=O)NC(C)C)ccc1-c1nc(Nc2ccc3[nH]ncc3c2)n(C)n1] |
| 259 | 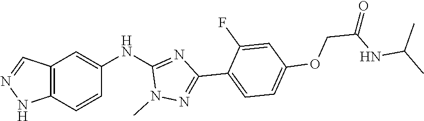 [SMILES: CC(C)NC(=O)COc1ccc(-c2nc(Nc3ccc4[nH]ncc4c3)n(C)n2)c(F)c1] |
| 260 | 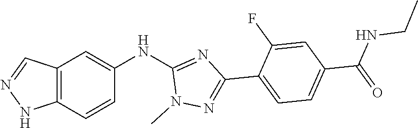 [SMILES: CCNC(=O)c1ccc(-c2nc(Nc3ccc4[nH]ncc4c3)n(C)n2)c(F)c1] |
| 261 | 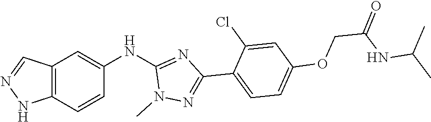 [SMILES: CC(C)NC(=O)COc1ccc(-c2nc(Nc3ccc4[nH]ncc4c3)n(C)n2)c(Cl)c1] |
| 262 | 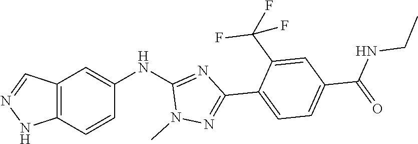 [SMILES: CCNC(=O)c1ccc(-c2nc(Nc3ccc4[nH]ncc4c3)n(C)n2)c(C(F)(F)F)c1] |
| 263 | 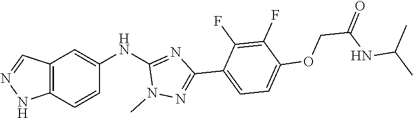 [SMILES: CC(C)NC(=O)COc1ccc(-c2nc(Nc3ccc4[nH]ncc4c3)n(C)n2)c(F)c1F] |
| 264 | 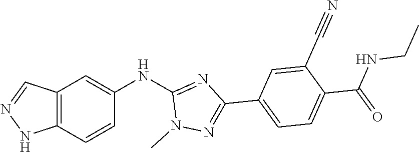 [SMILES: CCNC(=O)c1ccc(-c2nc(Nc3ccc4[nH]ncc4c3)n(C)n2)cc1C#N] |
| 265 | 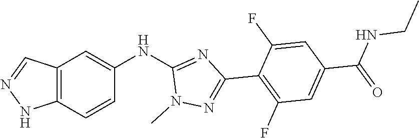 [SMILES: CCNC(=O)c1cc(F)c(-c2nc(Nc3ccc4[nH]ncc4c3)n(C)n2)c(F)c1] |
| 266 | 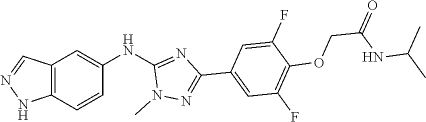 [SMILES: CC(C)NC(=O)COc1c(F)cc(-c2nc(Nc3ccc4[nH]ncc4c3)n(C)n2)cc1F] |
| 267 | 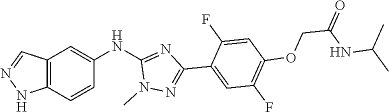 [SMILES: CC(C)NC(=O)COc1cc(F)c(-c2nc(Nc3ccc4[nH]ncc4c3)n(C)n2)cc1F] |
| 268 | 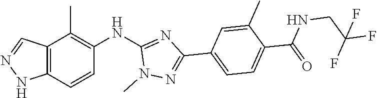 [SMILES: Cc1cc(-c2nc(Nc3ccc4[nH]ncc4c3C)n(C)n2)ccc1C(=O)NCC(F)(F)F] |
| 269 | 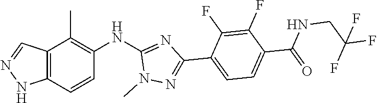 [SMILES: Cc1c(Nc2nc(-c3ccc(C(=O)NCC(F)(F)F)c(F)c3F)nn2C)ccc2[nH]ncc12] |
| 270 | 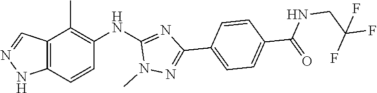 [SMILES: Cc1c(Nc2nc(-c3ccc(C(=O)NCC(F)(F)F)cc3)nn2C)ccc2[nH]ncc12] |
| 271 | 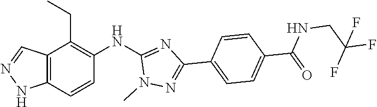 [SMILES: CCc1c(Nc2nc(-c3ccc(C(=O)NCC(F)(F)F)cc3)nn2C)ccc2[nH]ncc12] |
| 272 | 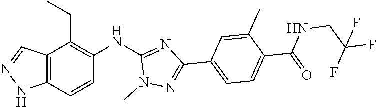 [SMILES: CCc1c(Nc2nc(-c3ccc(C(=O)NCC(F)(F)F)c(C)c3)nn2C)ccc2[nH]ncc12] |
| 273 | 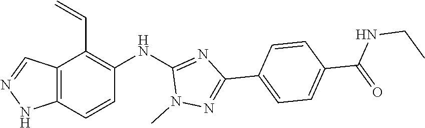 [SMILES: C=Cc1c(Nc2nc(-c3ccc(C(=O)NCC)cc3)nn2C)ccc2[nH]ncc12] |
| 274 | 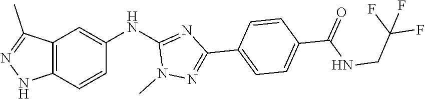 [SMILES: Cc1n[nH]c2ccc(Nc3nc(-c4ccc(C(=O)NCC(F)(F)F)cc4)nn3C)cc12] |
| 275 | 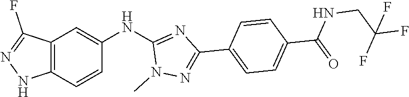 [SMILES: Cn1nc(-c2ccc(C(=O)NCC(F)(F)F)cc2)nc1Nc1ccc2[nH]nc(F)c2c1] |
| 276 | 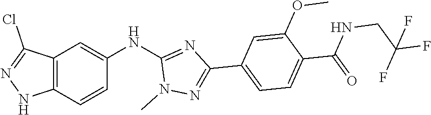 [SMILES: COc1cc(-c2nc(Nc3ccc4[nH]nc(Cl)c4c3)n(C)n2)ccc1C(=O)NCC(F)(F)F] |
| 277 | 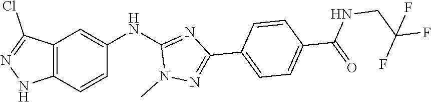 [SMILES: Cn1nc(-c2ccc(C(=O)NCC(F)(F)F)cc2)nc1Nc1ccc2[nH]nc(Cl)c2c1] |
| 278 | 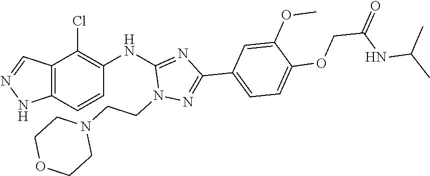 [SMILES: COc1cc(-c2nc(Nc3ccc4[nH]ncc4c3Cl)n(CCN3CCOCC3)n2)ccc1OCC(=O)NC(C)C] |
| 279 | 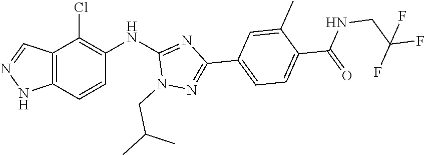 [SMILES: Cc1cc(-c2nc(Nc3ccc4[nH]ncc4c3Cl)n(CC(C)C)n2)ccc1C(=O)NCC(F)(F)F] |
| 280 | 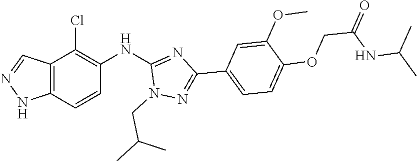 [SMILES: COc1cc(-c2nc(Nc3ccc4[nH]ncc4c3Cl)n(CC(C)C)n2)ccc1OCC(=O)NC(C)C] |
| 281 | 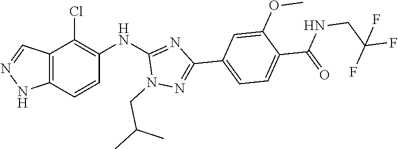 [SMILES: COc1cc(-c2nc(Nc3ccc4[nH]ncc4c3Cl)n(CC(C)C)n2)ccc1C(=O)NCC(F)(F)F] |
| 282 | 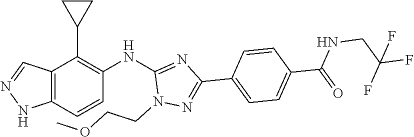 [SMILES: COCCn1nc(-c2ccc(C(=O)NCC(F)(F)F)cc2)nc1Nc1ccc2[nH]ncc2c1C1CC1] |
| 283 | 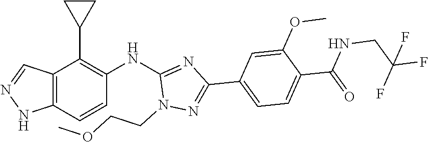 [SMILES: COCCn1nc(-c2ccc(C(=O)NCC(F)(F)F)c(OC)c2)nc1Nc1ccc2[nH]ncc2c1C1CC1] |
| 284 | 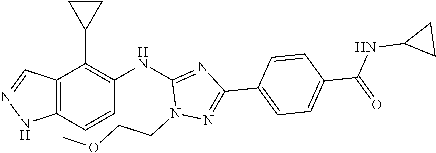 [SMILES: COCCn1nc(-c2ccc(C(=O)NC3CC3)cc2)nc1Nc1ccc2[nH]ncc2c1C1CC1] |
| 285 | 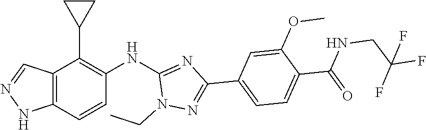 [SMILES: CCn1nc(-c2ccc(C(=O)NCC(F)(F)F)c(OC)c2)nc1Nc1ccc2[nH]ncc2c1C1CC1] |
| 286 | 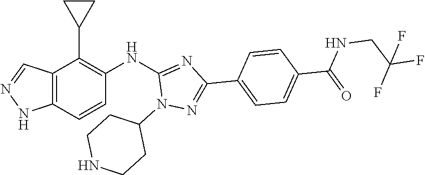 [SMILES: O=C(NCC(F)(F)F)c1ccc(-c2nc(Nc3ccc4[nH]ncc4c3C3CC3)n(C3CCNCC3)n2)cc1] |
| 287 | 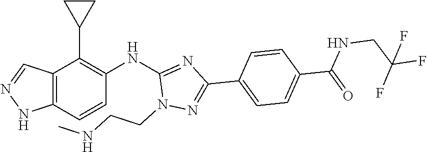 [SMILES: CNCCn1nc(-c2ccc(C(=O)NCC(F)(F)F)cc2)nc1Nc1ccc2[nH]ncc2c1C1CC1] |
| 288 | 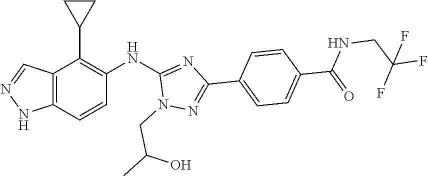 [SMILES: CC(O)Cn1nc(-c2ccc(C(=O)NCC(F)(F)F)cc2)nc1Nc1ccc2[nH]ncc2c1C1CC1] |
| 289 | 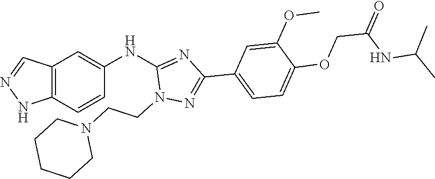 [SMILES: COc1cc(-c2nc(Nc3ccc4[nH]ncc4c3)n(CCN3CCCCC3)n2)ccc1OCC(=O)NC(C)C] |
| 290 | 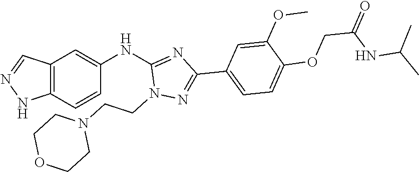 [SMILES: COc1cc(-c2nc(Nc3ccc4[nH]ncc4c3)n(CCN3CCOCC3)n2)ccc1OCC(=O)NC(C)C] |
| 291 | 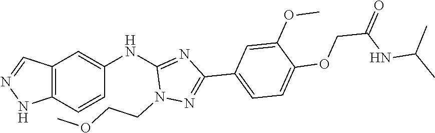 [SMILES: COCCn1nc(-c2ccc(OCC(=O)NC(C)C)c(OC)c2)nc1Nc1ccc2[nH]ncc2c1] |
| 292 | 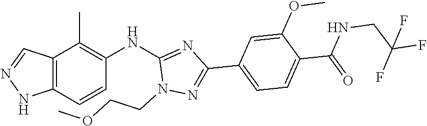 [SMILES: COCCn1nc(-c2ccc(C(=O)NCC(F)(F)F)c(OC)c2)nc1Nc1ccc2[nH]ncc2c1C] |
| 293 | 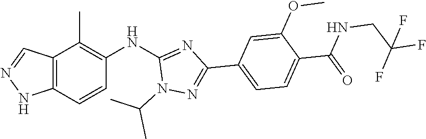 [SMILES: COc1cc(-c2nc(Nc3ccc4[nH]ncc4c3C)n(C(C)C)n2)ccc1C(=O)NCC(F)(F)F] |
| 294 | 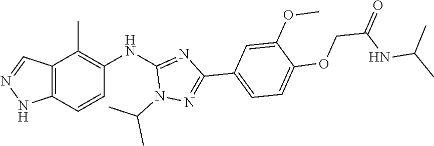 [SMILES: COc1cc(-c2nc(Nc3ccc4[nH]ncc4c3C)n(C(C)C)n2)ccc1OCC(=O)NC(C)C] |
| 295 | 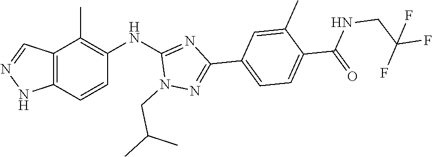 [SMILES: Cc1cc(-c2nc(Nc3ccc4[nH]ncc4c3C)n(CC(C)C)n2)ccc1C(=O)NCC(F)(F)F] |
| 296 | 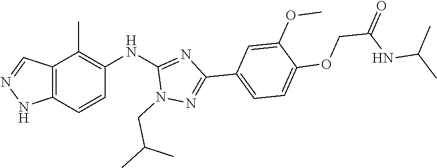 [SMILES: COc1cc(-c2nc(Nc3ccc4[nH]ncc4c3C)n(CC(C)C)n2)ccc1OCC(=O)NC(C)C] |
| 297 | 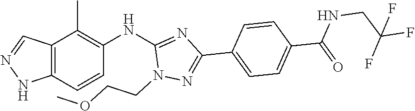 [SMILES: COCCn1nc(-c2ccc(C(=O)NCC(F)(F)F)cc2)nc1Nc1ccc2[nH]ncc2c1C] |
| 298 | 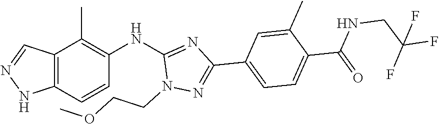 [SMILES: COCCn1nc(-c2ccc(C(=O)NCC(F)(F)F)c(C)c2)nc1Nc1ccc2[nH]ncc2c1C] |
| 299 | 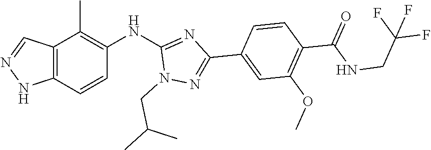 [SMILES: COc1cc(-c2nc(Nc3ccc4[nH]ncc4c3C)n(CC(C)C)n2)ccc1C(=O)NCC(F)(F)F] |
| 300 | 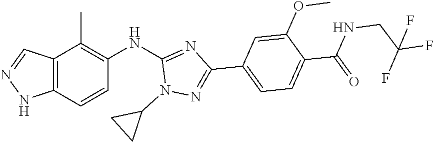 [SMILES: COc1cc(-c2nc(Nc3ccc4[nH]ncc4c3C)n(C3CC3)n2)ccc1C(=O)NCC(F)(F)F] |
| 301 | 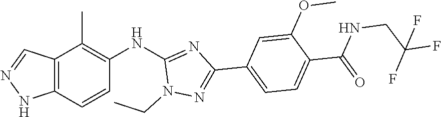 [SMILES: CCn1nc(-c2ccc(C(=O)NCC(F)(F)F)c(OC)c2)nc1Nc1ccc2[nH]ncc2c1C] |
| 302 | 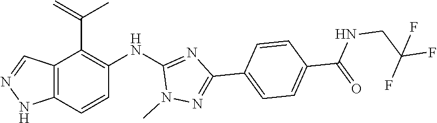 [SMILES: C=C(C)c1c(Nc2nc(-c3ccc(C(=O)NCC(F)(F)F)cc3)nn2C)ccc2[nH]ncc12] |
| 303 | 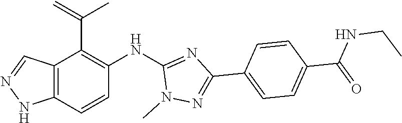 [SMILES: C=C(C)c1c(Nc2nc(-c3ccc(C(=O)NCC)cc3)nn2C)ccc2[nH]ncc12] |
| 304 | 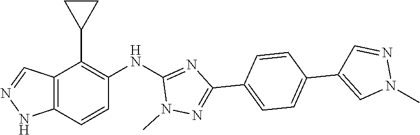 [SMILES: Cn1cc(-c2ccc(-c3nc(Nc4ccc5[nH]ncc5c4C4CC4)n(C)n3)cc2)cn1] |
| 305 | 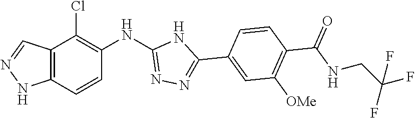 [SMILES: COc1cc(-c2nnc(Nc3ccc4[nH]ncc4c3Cl)[nH]2)ccc1C(=O)NCC(F)(F)F] |
| 306 | 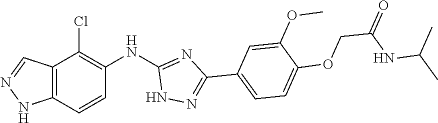 [SMILES: COc1cc(-c2n[nH]c(Nc3ccc4[nH]ncc4c3Cl)n2)ccc1OCC(=O)NC(C)C] |
| 307 | 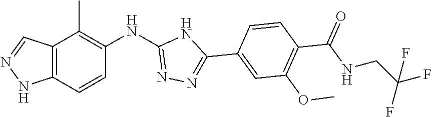 [SMILES: COc1cc(-c2nnc(Nc3ccc4[nH]ncc4c3C)[nH]2)ccc1C(=O)NCC(F)(F)F] |
| 308 | 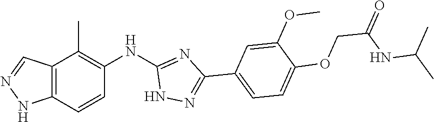 [SMILES: COc1cc(-c2n[nH]c(Nc3ccc4[nH]ncc4c3C)n2)ccc1OCC(=O)NC(C)C] |
| 309 | 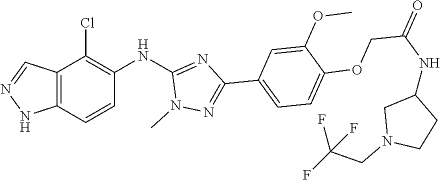 [SMILES: COc1cc(-c2nc(Nc3ccc4[nH]ncc4c3Cl)n(C)n2)ccc1OCC(=O)NC1CCN(CC(F)(F)F)C1] |
| 310 | 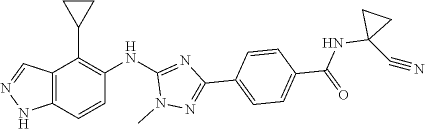 [SMILES: Cn1nc(-c2ccc(C(=O)NC3(C#N)CC3)cc2)nc1Nc1ccc2[nH]ncc2c1C1CC1] |
| 311 | 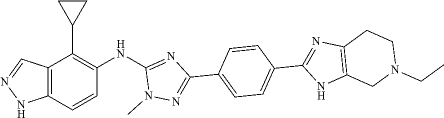 [SMILES: CCN1CCc2nc(-c3ccc(-c4nc(Nc5ccc6[nH]ncc6c5C5CC5)n(C)n4)cc3)[nH]c2C1] |
| 312 | 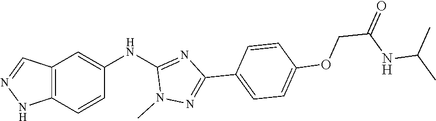 [SMILES: CC(C)NC(=O)COc1ccc(-c2nc(Nc3ccc4[nH]ncc4c3)n(C)n2)cc1] |
| 313 | 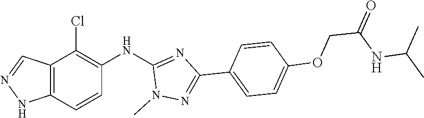 [SMILES: CC(C)NC(=O)COc1ccc(-c2nc(Nc3ccc4[nH]ncc4c3Cl)n(C)n2)cc1] |
| 314 | 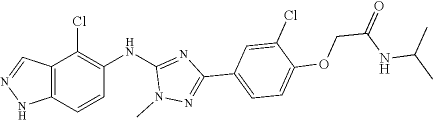 [SMILES: CC(C)NC(=O)COc1ccc(-c2nc(Nc3ccc4[nH]ncc4c3Cl)n(C)n2)cc1Cl] |
| 315 | 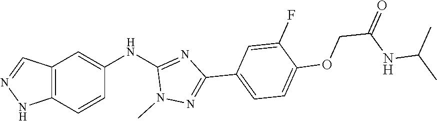 [SMILES: CC(C)NC(=O)COc1ccc(-c2nc(Nc3ccc4[nH]ncc4c3)n(C)n2)cc1F] |
| 316 | 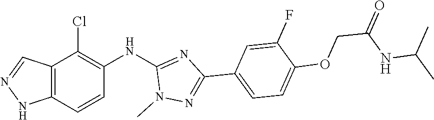 [SMILES: CC(C)NC(=O)COc1ccc(-c2nc(Nc3ccc4[nH]ncc4c3Cl)n(C)n2)cc1F] |
| 317 | 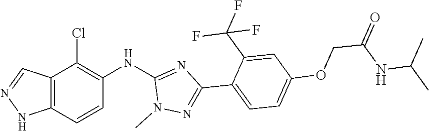 [SMILES: CC(C)NC(=O)COc1ccc(-c2nc(Nc3ccc4[nH]ncc4c3Cl)n(C)n2)c(C(F)(F)F)c1] |

In some embodiments, the selective ROCK2 inhibitor comprises Compound 1. In some embodiments, the selective ROCK2 inhibitor comprises Compound 2. In some embodiments, the selective ROCK2 inhibitor comprises Compound 3. In some embodiments, the selective ROCK2 inhibitor comprises Compound 4. In some embodiments, the selective ROCK2 inhibitor comprises Compound 5. In some embodiments, the selective ROCK2 inhibitor comprises Compound 6. In some embodiments, the selective ROCK2 inhibitor comprises Compound 7. In some embodiments, the selective ROCK2 inhibitor comprises Compound 8. In some embodiments, the selective ROCK2 inhibitor comprises Compound 9. In some embodiments, the selective ROCK2 inhibitor comprises Compound 10. In some embodiments, the selective ROCK2 inhibitor comprises Compound 11. In some embodiments, the selective ROCK2 inhibitor comprises Compound 12. In some embodiments, the selective ROCK2 inhibitor comprises Compound 13. In some embodiments, the selective ROCK2 inhibitor comprises Compound 14. In some embodiments, the selective ROCK2 inhibitor comprises Compound 15. In some embodiments, the selective ROCK2 inhibitor comprises Compound 16. In some embodiments, the selective ROCK2 inhibitor comprises Compound 17. In some embodiments, the selective ROCK2 inhibitor comprises Compound 18. In some embodiments, the selective ROCK2 inhibitor comprises Compound 19. In some embodiments, the selective ROCK2 inhibitor comprises Compound 20. In some embodiments, the selective ROCK2 inhibitor comprises Compound 21. In some embodiments, the selective ROCK2 inhibitor comprises Compound 22. In some embodiments, the selective ROCK2 inhibitor comprises Compound 23. In some embodiments, the selective ROCK2 inhibitor comprises Compound 24. In some embodiments, the selective ROCK2 inhibitor comprises Compound 25. In some embodiments, the selective ROCK2 inhibitor comprises Compound 26. In some embodiments, the selective ROCK2 inhibitor comprises Compound 27. In some embodiments, the selective ROCK2 inhibitor comprises Compound 28. In some embodiments, the selective ROCK2 inhibitor comprises Compound 29. In some embodiments, the selective ROCK2 inhibitor comprises Compound 30. In some embodiments, the selective ROCK2 inhibitor comprises Compound 31. In some embodiments, the selective ROCK2 inhibitor comprises Compound 32. In some embodiments, the selective ROCK2 inhibitor comprises Compound 33. In some embodiments, the selective ROCK2 inhibitor comprises Compound 34. In some embodiments, the selective ROCK2 inhibitor comprises Compound 35. In some embodiments, the selective ROCK2 inhibitor comprises Compound 36. In some embodiments, the selective ROCK2 inhibitor comprises Compound 37. In some embodiments, the selective ROCK2 inhibitor comprises Compound 38. In some embodiments, the selective ROCK2 inhibitor comprises Compound 39. In some embodiments, the selective ROCK2 inhibitor comprises Compound 40. In some embodiments, the selective ROCK2 inhibitor comprises Compound 41. In some embodiments, the selective ROCK2 inhibitor comprises Compound 42. In some embodiments, the selective ROCK2 inhibitor comprises Compound 43. In some embodiments, the selective ROCK2 inhibitor comprises Compound 44. In some embodiments, the selective ROCK2 inhibitor comprises Compound 45. In some embodiments, the selective ROCK2 inhibitor comprises Compound 46. In some embodiments, the selective ROCK2 inhibitor comprises Compound 47. In some embodiments, the selective ROCK2 inhibitor comprises Compound 48. In some embodiments, the selective ROCK2 inhibitor comprises Compound 49. In some embodiments, the selective ROCK2 inhibitor comprises Compound 50. In some embodiments, the selective ROCK2 inhibitor comprises Compound 51. In some embodiments, the selective ROCK2 inhibitor comprises Compound 52. In some embodiments, the selective ROCK2 inhibitor comprises Compound 53. In some embodiments, the selective ROCK2 inhibitor comprises Compound 54. In some embodiments, the selective ROCK2 inhibitor comprises Compound 55. In some embodiments, the selective ROCK2 inhibitor comprises Compound 56. In some embodiments, the selective ROCK2 inhibitor comprises Compound 57. In some embodiments, the selective ROCK2 inhibitor comprises Compound 58. In some embodiments, the selective ROCK2 inhibitor comprises Compound 59. In some embodiments, the selective ROCK2 inhibitor comprises Compound 60. In some embodiments, the selective ROCK2 inhibitor comprises Compound 61. In some embodiments, the selective ROCK2 inhibitor comprises Compound 62. In some embodiments, the selective ROCK2 inhibitor comprises Compound 63. In some embodiments, the selective ROCK2 inhibitor comprises Compound 64. In some embodiments, the selective ROCK2 inhibitor comprises Compound 65. In some embodiments, the selective ROCK2 inhibitor comprises Compound 66. In some embodiments, the selective ROCK2 inhibitor comprises Compound 67. In some embodiments, the selective ROCK2 inhibitor comprises Compound 68. In some embodiments, the selective ROCK2 inhibitor comprises Compound 69. In some embodiments, the selective ROCK2 inhibitor comprises Compound 70. In some embodiments, the selective ROCK2 inhibitor comprises Compound 71. In some embodiments, the selective ROCK2 inhibitor comprises Compound 72. In some embodiments, the selective ROCK2 inhibitor comprises Compound 73. In some embodiments, the selective ROCK2 inhibitor comprises Compound 74. In some embodiments, the selective ROCK2 inhibitor comprises Compound 75. In some embodiments, the selective ROCK2 inhibitor comprises Compound 76. In some embodiments, the selective ROCK2 inhibitor comprises Compound 77. In some embodiments, the selective ROCK2 inhibitor comprises Compound 78. In some embodiments, the selective ROCK2 inhibitor comprises Compound 79. In some embodiments, the selective ROCK2 inhibitor comprises Compound 80. In some embodiments, the selective ROCK2 inhibitor comprises Compound 81. In some embodiments, the selective ROCK2 inhibitor comprises Compound 82. In some embodiments, the selective ROCK2 inhibitor comprises Compound 83. In some embodiments, the selective ROCK2 inhibitor comprises Compound 84. In some embodiments, the selective ROCK2 inhibitor comprises Compound 85. In some embodiments, the selective ROCK2 inhibitor comprises Compound 86. In some embodiments, the selective ROCK2 inhibitor comprises Compound 87. In some embodiments, the selective ROCK2 inhibitor comprises Compound 88. In some embodiments, the selective ROCK2 inhibitor comprises Compound 89. In some embodiments, the selective ROCK2 inhibitor comprises Compound 90. In some embodiments, the selective ROCK2 inhibitor comprises Compound 91. In some embodiments, the selective ROCK2 inhibitor comprises Compound 92. In some embodiments, the selective ROCK2 inhibitor comprises Compound 93. In some embodiments, the selective ROCK2 inhibitor comprises Compound 94. In some embodiments, the selective ROCK2 inhibitor comprises Compound 95. In some embodiments, the selective ROCK2 inhibitor comprises Compound 96. In some embodiments, the selective ROCK2 inhibitor comprises Compound 97. In some embodiments, the selective ROCK2 inhibitor comprises Compound 98. In some embodiments, the selective ROCK2 inhibitor comprises Compound 99. In some embodiments, the selective ROCK2 inhibitor comprises Compound 100. In some embodiments, the selective ROCK2 inhibitor comprises Compound 101. In some embodiments, the selective ROCK2 inhibitor comprises Compound 102. In some embodiments, the selective ROCK2 inhibitor comprises Compound 103. In some embodiments, the selective ROCK2 inhibitor comprises Compound 104. In some embodiments, the selective ROCK2 inhibitor comprises Compound 105. In some embodiments, the selective ROCK2 inhibitor comprises Compound 106. In some embodiments, the selective ROCK2 inhibitor comprises Compound 107. In some embodiments, the selective ROCK2 inhibitor comprises Compound 108. In some embodiments, the selective ROCK2 inhibitor comprises Compound 109. In some embodiments, the selective ROCK2 inhibitor comprises Compound 110. In some embodiments, the selective ROCK2 inhibitor comprises Compound 111. In some embodiments, the selective ROCK2 inhibitor comprises Compound 112. In some embodiments, the selective ROCK2 inhibitor comprises Compound 113. In some embodiments, the selective ROCK2 inhibitor comprises Compound 114. In some embodiments, the selective ROCK2 inhibitor comprises Compound 115. In some embodiments, the selective ROCK2 inhibitor comprises Compound 116. In some embodiments, the selective ROCK2 inhibitor comprises Compound 117. In some embodiments, the selective ROCK2 inhibitor comprises Compound 118. In some embodiments, the selective ROCK2 inhibitor comprises Compound 119. In some embodiments, the selective ROCK2 inhibitor comprises Compound 120. In some embodiments, the selective ROCK2 inhibitor comprises Compound 121. In some embodiments, the selective ROCK2 inhibitor comprises Compound 122. In some embodiments, the selective ROCK2 inhibitor comprises Compound 123. In some embodiments, the selective ROCK2 inhibitor comprises Compound 124. In some embodiments, the selective ROCK2 inhibitor comprises Compound 125. In some embodiments, the selective ROCK2 inhibitor comprises Compound 126. In some embodiments, the selective ROCK2 inhibitor comprises Compound 127. In some embodiments, the selective ROCK2 inhibitor comprises Compound 128. In some embodiments, the selective ROCK2 inhibitor comprises Compound 129. In some embodiments, the selective ROCK2 inhibitor comprises Compound 130. In some embodiments, the selective ROCK2 inhibitor comprises Compound 131. In some embodiments, the selective ROCK2 inhibitor comprises Compound 132. In some embodiments, the selective ROCK2 inhibitor comprises Compound 133. In some embodiments, the selective ROCK2 inhibitor comprises Compound 134. In some embodiments, the selective ROCK2 inhibitor comprises Compound 135. In some embodiments, the selective ROCK2 inhibitor comprises Compound 136. In some embodiments, the selective ROCK2 inhibitor comprises Compound 137. In some embodiments, the selective ROCK2 inhibitor comprises Compound 138. In some embodiments, the selective ROCK2 inhibitor comprises Compound 139. In some embodiments, the selective ROCK2 inhibitor comprises Compound 140. In some embodiments, the selective ROCK2 inhibitor comprises Compound 141. In some embodiments, the selective ROCK2 inhibitor comprises Compound 142. In some embodiments, the selective ROCK2 inhibitor comprises Compound 143. In some embodiments, the selective ROCK2 inhibitor comprises Compound 144. In some embodiments, the selective ROCK2 inhibitor comprises Compound 145. In some embodiments, the selective ROCK2 inhibitor comprises Compound 146. In some embodiments, the selective ROCK2 inhibitor comprises Compound 147. In some embodiments, the selective ROCK2 inhibitor comprises Compound 148. In some embodiments, the selective ROCK2 inhibitor comprises Compound 149. In some embodiments, the selective ROCK2 inhibitor comprises Compound 150. In some embodiments, the selective ROCK2 inhibitor comprises Compound 151. In some embodiments, the selective ROCK2 inhibitor comprises Compound 152. In some embodiments, the selective ROCK2 inhibitor comprises Compound 153. In some embodiments, the selective ROCK2 inhibitor comprises Compound 154. In some embodiments, the selective ROCK2 inhibitor comprises Compound 155. In some embodiments, the selective ROCK2 inhibitor comprises Compound 156. In some embodiments, the selective ROCK2 inhibitor comprises Compound 157. In some embodiments, the selective ROCK2 inhibitor comprises Compound 158. In some embodiments, the selective ROCK2 inhibitor comprises Compound 159. In some embodiments, the selective ROCK2 inhibitor comprises Compound 160. In some embodiments, the selective ROCK2 inhibitor comprises Compound 161. In some embodiments, the selective ROCK2 inhibitor comprises Compound 162. In some embodiments, the selective ROCK2 inhibitor comprises Compound 163. In some embodiments, the selective ROCK2 inhibitor comprises Compound 164. In some embodiments, the selective ROCK2 inhibitor comprises Compound 165. In some embodiments, the selective ROCK2 inhibitor comprises Compound 166. In some embodiments, the selective ROCK2 inhibitor comprises Compound 167. In some embodiments, the selective ROCK2 inhibitor comprises Compound 168. In some embodiments, the selective ROCK2 inhibitor comprises Compound 169. In some embodiments, the selective ROCK2 inhibitor comprises Compound 170. In some embodiments, the selective ROCK2 inhibitor comprises Compound 171. In some embodiments, the selective ROCK2 inhibitor comprises Compound 172. In some embodiments, the selective ROCK2 inhibitor comprises Compound 173. In some embodiments, the selective ROCK2 inhibitor comprises Compound 174. In some embodiments, the selective ROCK2 inhibitor comprises Compound 175. In some embodiments, the selective ROCK2 inhibitor comprises Compound 176. In some embodiments, the selective ROCK2 inhibitor comprises Compound 177. In some embodiments, the selective ROCK2 inhibitor comprises Compound 178. In some embodiments, the selective ROCK2 inhibitor comprises Compound 179. In some embodiments, the selective ROCK2 inhibitor comprises Compound 180. In some embodiments, the selective ROCK2 inhibitor comprises Compound 181. In some embodiments, the selective ROCK2 inhibitor comprises Compound 182. In some embodiments, the selective ROCK2 inhibitor comprises Compound 183. In some embodiments, the selective ROCK2 inhibitor comprises Compound 184. In some embodiments, the selective ROCK2 inhibitor comprises Compound 185. In some embodiments, the selective ROCK2 inhibitor comprises Compound 186. In some embodiments, the selective ROCK2 inhibitor comprises Compound 187. In some embodiments, the selective ROCK2 inhibitor comprises Compound 188. In some embodiments, the selective ROCK2 inhibitor comprises Compound 189. In some embodiments, the selective ROCK2 inhibitor comprises Compound 190. In some embodiments, the selective ROCK2 inhibitor comprises Compound 191. In some embodiments, the selective ROCK2 inhibitor comprises Compound 192. In some embodiments, the selective ROCK2 inhibitor comprises Compound 193. In some embodiments, the selective ROCK2 inhibitor comprises Compound 194. In some embodiments, the selective ROCK2 inhibitor comprises Compound 195. In some embodiments, the selective ROCK2 inhibitor comprises Compound 196. In some embodiments, the selective ROCK2 inhibitor comprises Compound 197. In some embodiments, the selective ROCK2 inhibitor comprises Compound 198. In some embodiments, the selective ROCK2 inhibitor comprises Compound 199. In some embodiments, the selective ROCK2 inhibitor comprises Compound 200. In some embodiments, the selective ROCK2 inhibitor comprises Compound 201. In some embodiments, the selective ROCK2 inhibitor comprises Compound 202. In some embodiments, the selective ROCK2 inhibitor comprises Compound 203. In some embodiments, the selective ROCK2 inhibitor comprises Compound 204. In some embodiments, the selective ROCK2 inhibitor comprises Compound 205. In some embodiments, the selective ROCK2 inhibitor comprises Compound 206. In some embodiments, the selective ROCK2 inhibitor comprises Compound 207. In some embodiments, the selective ROCK2 inhibitor comprises Compound 208. In some embodiments, the selective ROCK2 inhibitor comprises Compound 209. In some embodiments, the selective ROCK2 inhibitor comprises Compound 210. In some embodiments, the selective ROCK2 inhibitor comprises Compound 211. In some embodiments, the selective ROCK2 inhibitor comprises Compound 212. In some embodiments, the selective ROCK2 inhibitor comprises Compound 213. In some embodiments, the selective ROCK2 inhibitor comprises Compound 214. In some embodiments, the selective ROCK2 inhibitor comprises Compound 215. In some embodiments, the selective ROCK2 inhibitor comprises Compound 216. In some embodiments, the selective ROCK2 inhibitor comprises Compound 217. In some embodiments, the selective ROCK2 inhibitor comprises Compound 218. In some embodiments, the selective ROCK2 inhibitor comprises Compound 219. In some embodiments, the selective ROCK2 inhibitor comprises Compound 220. In some embodiments, the selective ROCK2 inhibitor comprises Compound 221. In some embodiments, the selective ROCK2 inhibitor comprises Compound 222. In some embodiments, the selective ROCK2 inhibitor comprises Compound 223. In some embodiments, the selective ROCK2 inhibitor comprises Compound 224. In some embodiments, the selective ROCK2 inhibitor comprises Compound 225. In some embodiments, the selective ROCK2 inhibitor comprises Compound 226. In some embodiments, the selective ROCK2 inhibitor comprises Compound 227. In some embodiments, the selective ROCK2 inhibitor comprises Compound 228. In some embodiments, the selective ROCK2 inhibitor comprises Compound 229. In some embodiments, the selective ROCK2 inhibitor comprises Compound 230. In some embodiments, the selective ROCK2 inhibitor comprises Compound 231. In some embodiments, the selective ROCK2 inhibitor comprises Compound 232. In some embodiments, the selective ROCK2 inhibitor comprises Compound 233. In some embodiments, the selective ROCK2 inhibitor comprises Compound 234. In some embodiments, the selective ROCK2 inhibitor comprises Compound 235. In some embodiments, the selective ROCK2 inhibitor comprises Compound 236. In some embodiments, the selective ROCK2 inhibitor comprises Compound 237. In some embodiments, the selective ROCK2 inhibitor comprises Compound 238. In some embodiments, the selective ROCK2 inhibitor comprises Compound 239. In some embodiments, the selective ROCK2 inhibitor comprises Compound 240. In some embodiments, the selective ROCK2 inhibitor comprises Compound 241. In some embodiments, the selective ROCK2 inhibitor comprises Compound 242. In some embodiments, the selective ROCK2 inhibitor comprises Compound 243. In some embodiments, the selective ROCK2 inhibitor comprises Compound 244. In some embodiments, the selective ROCK2 inhibitor comprises Compound 245. In some embodiments, the selective ROCK2 inhibitor comprises Compound 246. In some embodiments, the selective ROCK2 inhibitor comprises Compound 247. In some embodiments, the selective ROCK2 inhibitor comprises Compound 248. In some embodiments, the selective ROCK2 inhibitor comprises Compound 249. In some embodiments, the selective ROCK2 inhibitor comprises Compound 250. In some embodiments, the selective ROCK2 inhibitor comprises Compound 251. In some embodiments, the selective ROCK2 inhibitor comprises Compound 252. In some embodiments, the selective ROCK2 inhibitor comprises Compound 253. In some embodiments, the selective ROCK2 inhibitor comprises Compound 254. In some embodiments, the selective ROCK2 inhibitor comprises Compound 255. In some embodiments, the selective ROCK2 inhibitor comprises Compound 256. In some embodiments, the selective ROCK2 inhibitor comprises Compound 257. In some embodiments, the selective ROCK2 inhibitor comprises Compound 258. In some embodiments, the selective ROCK2 inhibitor comprises Compound 259. In some embodiments, the selective ROCK2 inhibitor comprises Compound 260. In some embodiments, the selective ROCK2 inhibitor comprises Compound 261. In some embodiments, the selective ROCK2 inhibitor comprises Compound 262. In some embodiments, the selective ROCK2 inhibitor comprises Compound 263. In some embodiments, the selective ROCK2 inhibitor comprises Compound 264. In some embodiments, the selective ROCK2 inhibitor comprises Compound 265. In some embodiments, the selective ROCK2 inhibitor comprises Compound 266. In some embodiments, the selective ROCK2 inhibitor comprises Compound 267. In some embodiments, the selective ROCK2 inhibitor comprises Compound 268. In some embodiments, the selective ROCK2 inhibitor comprises Compound 269. In some embodiments, the selective ROCK2 inhibitor comprises Compound 270. In some embodiments, the selective ROCK2 inhibitor comprises Compound 271. In some embodiments, the selective ROCK2 inhibitor comprises Compound 272. In some embodiments, the selective ROCK2 inhibitor comprises Compound 273. In some embodiments, the selective ROCK2 inhibitor comprises Compound 274. In some embodiments, the selective ROCK2 inhibitor comprises Compound 275. In some embodiments, the selective ROCK2 inhibitor comprises Compound 276. In some embodiments, the selective ROCK2 inhibitor comprises Compound 277. In some embodiments, the selective ROCK2 inhibitor comprises Compound 278. In some embodiments, the selective ROCK2 inhibitor comprises Compound 279. In some embodiments, the selective ROCK2 inhibitor comprises Compound 280. In some embodiments, the selective ROCK2 inhibitor comprises Compound 281. In some embodiments, the selective ROCK2 inhibitor comprises Compound 282. In some embodiments, the selective ROCK2 inhibitor comprises Compound 283. In some embodiments, the selective ROCK2 inhibitor comprises Compound 284. In some embodiments, the selective ROCK2 inhibitor comprises Compound 285. In some embodiments, the selective ROCK2 inhibitor comprises Compound 286. In some embodiments, the selective ROCK2 inhibitor comprises Compound 287. In some embodiments, the selective ROCK2 inhibitor comprises Compound 288. In some embodiments, the selective ROCK2 inhibitor comprises Compound 289. In some embodiments, the selective ROCK2 inhibitor comprises Compound 290. In some embodiments, the selective ROCK2 inhibitor comprises Compound 291. In some embodiments, the selective ROCK2 inhibitor comprises Compound 292. In some embodiments, the selective ROCK2 inhibitor comprises Compound 293. In some embodiments, the selective ROCK2 inhibitor comprises Compound 294. In some embodiments, the selective ROCK2 inhibitor comprises Compound 295. In some embodiments, the selective ROCK2 inhibitor comprises Compound 296. In some embodiments, the selective ROCK2 inhibitor comprises Compound 297. In some embodiments, the selective ROCK2 inhibitor comprises Compound 298. In some embodiments, the selective ROCK2 inhibitor comprises Compound 299. In some embodiments, the selective ROCK2 inhibitor comprises Compound 300. In some embodiments, the selective ROCK2 inhibitor comprises Compound 301. In some embodiments, the selective ROCK2 inhibitor comprises Compound 302. In some embodiments, the selective ROCK2 inhibitor comprises Compound 303. In some embodiments, the selective ROCK2 inhibitor comprises Compound 304. In some embodiments, the selective ROCK2 inhibitor comprises Compound 305. In some embodiments, the selective ROCK2 inhibitor comprises Compound 306. In some embodiments, the selective ROCK2 inhibitor comprises Compound 307. In some embodiments, the selective ROCK2 inhibitor comprises Compound 308. In some embodiments, the selective ROCK2 inhibitor comprises Compound 309. In some embodiments, the selective ROCK2 inhibitor comprises Compound 310. In some embodiments, the selective ROCK2 inhibitor comprises Compound 311. In some embodiments, the selective ROCK2 inhibitor comprises Compound 312. In some embodiments, the selective ROCK2 inhibitor comprises Compound 313. In some embodiments, the selective ROCK2 inhibitor comprises Compound 314. In some embodiments, the selective ROCK2 inhibitor comprises Compound 315. In some embodiments, the selective ROCK2 inhibitor comprises Compound 316. In some embodiments, the selective ROCK2 inhibitor comprises Compound 317.

In some embodiments, the selective ROCK2 inhibitor is Compound 1. In some embodiments, the selective ROCK2 inhibitor is Compound 2. In some embodiments, the selective ROCK2 inhibitor is Compound 3. In some embodiments, the selective ROCK2 inhibitor is a hydrochloride salt of Compound 3. In some embodiments, the selective ROCK2 inhibitor is the dihydrochloride salt of Compound 3. In some embodiments, the selective ROCK2 inhibitor is Compound 4. In some embodiments, the selective ROCK2 inhibitor is Compound 5. In some embodiments, the selective ROCK2 inhibitor is Compound 6. In some embodiments, the selective ROCK2 inhibitor is Compound 7. In some embodiments, the selective ROCK2 inhibitor is Compound 8. In some embodiments, the selective ROCK2 inhibitor is Compound 9. In some embodiments, the selective ROCK2 inhibitor is the hydrochloride salt of Compound 9. In some embodiments, the selective ROCK2 inhibitor is Compound 10. In some embodiments, the selective ROCK2 inhibitor is Compound 11. In some embodiments, the selective ROCK2 inhibitor is Compound 12. In some embodiments, the selective ROCK2 inhibitor is Compound 13. In some embodiments, the selective ROCK2 inhibitor is Compound 14. In some embodiments, the selective ROCK2 inhibitor is Compound 15. In some embodiments, the selective ROCK2 inhibitor is Compound 16. In some embodiments, the selective ROCK2 inhibitor is Compound 17. In some embodiments, the selective ROCK2 inhibitor is Compound 18. In some embodiments, the selective ROCK2 inhibitor is Compound 19. In some embodiments, the selective ROCK2 inhibitor is Compound 20. In some embodiments, the selective ROCK2 inhibitor is Compound 21. In some embodiments, the selective ROCK2 inhibitor is Compound 22. In some embodiments, the selective ROCK2 inhibitor is Compound 23. In some embodiments, the selective ROCK2 inhibitor is Compound 24. In some embodiments, the selective ROCK2 inhibitor is Compound 25. In some embodiments, the selective ROCK2 inhibitor is Compound 26. In some embodiments, the selective ROCK2 inhibitor is Compound 27. In some embodiments, the selective ROCK2 inhibitor is Compound 28. In some embodiments, the selective ROCK2 inhibitor is Compound 29. In some embodiments, the selective ROCK2 inhibitor is Compound 30. In some embodiments, the selective ROCK2 inhibitor is Compound 31. In some embodiments, the selective ROCK2 inhibitor is Compound 32. In some embodiments, the selective ROCK2 inhibitor is Compound 33. In some embodiments, the selective ROCK2 inhibitor is Compound 34. In some embodiments, the selective ROCK2 inhibitor is Compound 35. In some embodiments, the selective ROCK2 inhibitor is Compound 36. In some embodiments, the selective ROCK2 inhibitor is Compound 37. In some embodiments, the selective ROCK2 inhibitor is Compound 38. In some embodiments, the selective ROCK2 inhibitor is Compound 39. In some embodiments, the selective ROCK2 inhibitor is Compound 40. In some embodiments, the selective ROCK2 inhibitor is Compound 41. In some embodiments, the selective ROCK2 inhibitor is Compound 42. In some embodiments, the selective ROCK2 inhibitor is Compound 43. In some embodiments, the selective ROCK2 inhibitor is Compound 44. In some embodiments, the selective ROCK2 inhibitor is Compound 45. In some embodiments, the selective ROCK2 inhibitor is Compound 46. In some embodiments, the selective ROCK2 inhibitor is Compound 47. In some embodiments, the selective ROCK2 inhibitor is Compound 48. In some embodiments, the selective ROCK2 inhibitor is Compound 49. In some embodiments, the selective ROCK2 inhibitor is Compound 50. In some embodiments, the selective ROCK2 inhibitor is Compound 51. In some embodiments, the selective ROCK2 inhibitor is Compound 52. In some embodiments, the selective ROCK2 inhibitor is Compound 53. In some embodiments, the selective ROCK2 inhibitor is Compound 54. In some embodiments, the selective ROCK2 inhibitor is Compound 55. In some embodiments, the selective ROCK2 inhibitor is Compound 56. In some embodiments, the selective ROCK2 inhibitor is Compound 57. In some embodiments, the selective ROCK2 inhibitor is Compound 58. In some embodiments, the selective ROCK2 inhibitor is Compound 59. In some embodiments, the selective ROCK2 inhibitor is Compound 60. In some embodiments, the selective ROCK2 inhibitor is Compound 61. In some embodiments, the selective ROCK2 inhibitor is Compound 62. In some embodiments, the selective ROCK2 inhibitor is Compound 63. In some embodiments, the selective ROCK2 inhibitor is Compound 64. In some embodiments, the selective ROCK2 inhibitor is Compound 65. In some embodiments, the selective ROCK2 inhibitor is Compound 66. In some embodiments, the selective ROCK2 inhibitor is Compound 67. In some embodiments, the selective ROCK2 inhibitor is Compound 68. In some embodiments, the selective ROCK2 inhibitor is Compound 69. In some embodiments, the selective ROCK2 inhibitor is Compound 70. In some embodiments, the selective ROCK2 inhibitor is Compound 71. In some embodiments, the selective ROCK2 inhibitor is Compound 72. In some embodiments, the selective ROCK2 inhibitor is Compound 73. In some embodiments, the selective ROCK2 inhibitor is the hydrochloride salt of Compound 73. In some embodiments, the selective ROCK2 inhibitor is Compound 74. In some embodiments, the selective ROCK2 inhibitor is Compound 75. In some embodiments, the selective ROCK2 inhibitor is Compound 76. In some embodiments, the selective ROCK2 inhibitor is Compound 77. In some embodiments, the selective ROCK2 inhibitor is Compound 78. In some embodiments, the selective ROCK2 inhibitor is Compound 79. In some embodiments, the selective ROCK2 inhibitor is Compound 80. In some embodiments, the selective ROCK2 inhibitor is Compound 81. In some embodiments, the selective ROCK2 inhibitor is Compound 82. In some embodiments, the selective ROCK2 inhibitor is Compound 83. In some embodiments, the selective ROCK2 inhibitor is Compound 84. In some embodiments, the selective ROCK2 inhibitor is Compound 85. In some embodiments, the selective ROCK2 inhibitor is Compound 86. In some embodiments, the selective ROCK2 inhibitor is Compound 87. In some embodiments, the selective ROCK2 inhibitor is Compound 88. In some embodiments, the selective ROCK2 inhibitor is Compound 89. In some embodiments, the selective ROCK2 inhibitor is Compound 90. In some embodiments, the selective ROCK2 inhibitor is Compound 91. In some embodiments, the selective ROCK2 inhibitor is Compound 92. In some embodiments, the selective ROCK2 inhibitor is Compound 93. In some embodiments, the selective ROCK2 inhibitor is Compound 94. In some embodiments, the selective ROCK2 inhibitor is Compound 95. In some embodiments, the selective ROCK2 inhibitor is Compound 96. In some embodiments, the selective ROCK2 inhibitor is Compound 97. In some embodiments, the selective ROCK2 inhibitor is Compound 98. In some embodiments, the selective ROCK2 inhibitor is Compound 99. In some embodiments, the selective ROCK2 inhibitor is Compound 100. In some embodiments, the selective ROCK2 inhibitor is Compound 101. In some embodiments, the selective ROCK2 inhibitor is Compound 102. In some embodiments, the selective ROCK2 inhibitor is Compound 103. In some embodiments, the selective ROCK2 inhibitor is Compound 104. In some embodiments, the selective ROCK2 inhibitor is Compound 105. In some embodiments, the selective ROCK2 inhibitor is Compound 106. In some embodiments, the selective ROCK2 inhibitor is Compound 107. In some embodiments, the selective ROCK2 inhibitor is Compound 108. In some embodiments, the selective ROCK2 inhibitor is Compound 109. In some embodiments, the selective ROCK2 inhibitor is Compound 110. In some embodiments, the selective ROCK2 inhibitor is Compound 111. In some embodiments, the selective ROCK2 inhibitor is Compound 112. In some embodiments, the selective ROCK2 inhibitor is Compound 113. In some embodiments, the selective ROCK2 inhibitor is Compound 114. In some embodiments, the selective ROCK2 inhibitor is Compound 115. In some embodiments, the selective ROCK2 inhibitor is Compound 116. In some embodiments, the selective ROCK2 inhibitor is Compound 117. In some embodiments, the selective ROCK2 inhibitor is Compound 118. In some embodiments, the selective ROCK2 inhibitor is Compound 119. In some embodiments, the selective ROCK2 inhibitor is Compound 120. In some embodiments, the selective ROCK2 inhibitor is Compound 121. In some embodiments, the selective ROCK2 inhibitor is Compound 122. In some embodiments, the selective ROCK2 inhibitor is Compound 123. In some embodiments, the selective ROCK2 inhibitor is Compound 124. In some embodiments, the selective ROCK2 inhibitor is Compound 125. In some embodiments, the selective ROCK2 inhibitor is Compound 126. In some embodiments, the selective ROCK2 inhibitor is Compound 127. In some embodiments, the selective ROCK2 inhibitor is Compound 128. In some embodiments, the selective ROCK2 inhibitor is a hydrochloride salt of Compound 128. In some embodiments, the selective ROCK2 inhibitor is the dihydrochloride salt of Compound 128. In some embodiments, the selective ROCK2 inhibitor is Compound 129. In some embodiments, the selective ROCK2 inhibitor is Compound 130. In some embodiments, the selective ROCK2 inhibitor is Compound 131. In some embodiments, the selective ROCK2 inhibitor is Compound 132. In some embodiments, the selective ROCK2 inhibitor is Compound 133. In some embodiments, the selective ROCK2 inhibitor is Compound 134. In some embodiments, the selective ROCK2 inhibitor is Compound 135. In some embodiments, the selective ROCK2 inhibitor is Compound 136. In some embodiments, the selective ROCK2 inhibitor is Compound 137. In some embodiments, the selective ROCK2 inhibitor is Compound 138. In some embodiments, the selective ROCK2 inhibitor is Compound 139. In some embodiments, the selective ROCK2 inhibitor is Compound 140. In some embodiments, the selective ROCK2 inhibitor is Compound 141. In some embodiments, the selective ROCK2 inhibitor is Compound 142. In some embodiments, the selective ROCK2 inhibitor is Compound 143. In some embodiments, the selective ROCK2 inhibitor is Compound 144. In some embodiments, the selective ROCK2 inhibitor is Compound 145. In some embodiments, the selective ROCK2 inhibitor is Compound 146. In some embodiments, the selective ROCK2 inhibitor is Compound 147. In some embodiments, the selective ROCK2 inhibitor is Compound 148. In some embodiments, the selective ROCK2 inhibitor is Compound 149. In some embodiments, the selective ROCK2 inhibitor is Compound 150. In some embodiments, the selective ROCK2 inhibitor is Compound 151. In some embodiments, the selective ROCK2 inhibitor is Compound 152. In some embodiments, the selective ROCK2 inhibitor is Compound 153. In some embodiments, the selective ROCK2 inhibitor is Compound 154. In some embodiments, the selective ROCK2 inhibitor is Compound 155. In some embodiments, the selective ROCK2 inhibitor is Compound 156. In some embodiments, the selective ROCK2 inhibitor is Compound 157. In some embodiments, the selective ROCK2 inhibitor is Compound 158. In some embodiments, the selective ROCK2 inhibitor is Compound 159. In some embodiments, the selective ROCK2 inhibitor is Compound 160. In some embodiments, the selective ROCK2 inhibitor is Compound 161. In some embodiments, the selective ROCK2 inhibitor is Compound 162. In some embodiments, the selective ROCK2 inhibitor is Compound 163. In some embodiments, the selective ROCK2 inhibitor is Compound 164. In some embodiments, the selective ROCK2 inhibitor is Compound 165. In some embodiments, the selective ROCK2 inhibitor is Compound 166. In some embodiments, the selective ROCK2 inhibitor is Compound 167. In some embodiments, the selective ROCK2 inhibitor is Compound 168. In some embodiments, the selective ROCK2 inhibitor is Compound 169. In some embodiments, the selective ROCK2 inhibitor is Compound 170. In some embodiments, the selective ROCK2 inhibitor is Compound 171. In some embodiments, the selective ROCK2 inhibitor is Compound 172. In some embodiments, the selective ROCK2 inhibitor is Compound 173. In some embodiments, the selective ROCK2 inhibitor is Compound 174. In some embodiments, the selective ROCK2 inhibitor is Compound 175. In some embodiments, the selective ROCK2 inhibitor is Compound 176. In some embodiments, the selective ROCK2 inhibitor is Compound 177. In some embodiments, the selective ROCK2 inhibitor is Compound 178. In some embodiments, the selective ROCK2 inhibitor is Compound 179. In some embodiments, the selective ROCK2 inhibitor is Compound 180. In some embodiments, the selective ROCK2 inhibitor is Compound 181. In some embodiments, the selective ROCK2 inhibitor is Compound 182. In some embodiments, the selective ROCK2 inhibitor is Compound 183. In some embodiments, the selective ROCK2 inhibitor is Compound 184. In some embodiments, the selective ROCK2 inhibitor is Compound 185. In some embodiments, the selective ROCK2 inhibitor is Compound 186. In some embodiments, the selective ROCK2 inhibitor is Compound 187. In some embodiments, the selective ROCK2 inhibitor is Compound 188. In some embodiments, the selective ROCK2 inhibitor is Compound 189. In some embodiments, the selective ROCK2 inhibitor is Compound 190. In some embodiments, the selective ROCK2 inhibitor is Compound 191. In some embodiments, the selective ROCK2 inhibitor is Compound 192. In some embodiments, the selective ROCK2 inhibitor is Compound 193. In some embodiments, the selective ROCK2 inhibitor is Compound 194. In some embodiments, the selective ROCK2 inhibitor is Compound 195. In some embodiments, the selective ROCK2 inhibitor is Compound 196. In some embodiments, the selective ROCK2 inhibitor is Compound 197. In some embodiments, the selective ROCK2 inhibitor is Compound 198. In some embodiments, the selective ROCK2 inhibitor is Compound 199. In some embodiments, the selective ROCK2 inhibitor is Compound 200. In some embodiments, the selective ROCK2 inhibitor is Compound 201. In some embodiments, the selective ROCK2 inhibitor is Compound 202. In some embodiments, the selective ROCK2 inhibitor is Compound 203. In some embodiments, the selective ROCK2 inhibitor is Compound 204. In some embodiments, the selective ROCK2 inhibitor is Compound 205. In some embodiments, the selective ROCK2 inhibitor is Compound 206. In some embodiments, the selective ROCK2 inhibitor is Compound 207. In some embodiments, the selective ROCK2 inhibitor is Compound 208. In some embodiments, the selective ROCK2 inhibitor is Compound 209. In some embodiments, the selective ROCK2 inhibitor is Compound 210. In some embodiments, the selective ROCK2 inhibitor is Compound 211. In some embodiments, the selective ROCK2 inhibitor is Compound 212. In some embodiments, the selective ROCK2 inhibitor is Compound 213. In some embodiments, the selective ROCK2 inhibitor is Compound 214. In some embodiments, the selective ROCK2 inhibitor is Compound 215. In some embodiments, the selective ROCK2 inhibitor is Compound 216. In some embodiments, the selective ROCK2 inhibitor is Compound 217. In some embodiments, the selective ROCK2 inhibitor is Compound 218. In some embodiments, the selective ROCK2 inhibitor is Compound 219. In some embodiments, the selective ROCK2 inhibitor is Compound 220. In some embodiments, the selective ROCK2 inhibitor is Compound 221. In some embodiments, the selective ROCK2 inhibitor is Compound 222. In some embodiments, the selective ROCK2 inhibitor is Compound 223. In some embodiments, the selective ROCK2 inhibitor is Compound 224. In some embodiments, the selective ROCK2 inhibitor is Compound 225. In some embodiments, the selective ROCK2 inhibitor is Compound 226. In some embodiments, the selective ROCK2 inhibitor is Compound 227. In some embodiments, the selective ROCK2 inhibitor is Compound 228. In some embodiments, the selective ROCK2 inhibitor is Compound 229. In some embodiments, the selective ROCK2 inhibitor is Compound 230. In some embodiments, the selective ROCK2 inhibitor is Compound 231. In some embodiments, the selective ROCK2 inhibitor is Compound 232. In some embodiments, the selective ROCK2 inhibitor is Compound 233. In some embodiments, the selective ROCK2 inhibitor is Compound 234. In some embodiments, the selective ROCK2 inhibitor is Compound 235. In some embodiments, the selective ROCK2 inhibitor is Compound 236. In some embodiments, the selective ROCK2 inhibitor is Compound 237. In some embodiments, the selective ROCK2 inhibitor is Compound 238. In some embodiments, the selective ROCK2 inhibitor is Compound 239. In some embodiments, the selective ROCK2 inhibitor is Compound 240. In some embodiments, the selective ROCK2 inhibitor is Compound 241. In some embodiments, the selective ROCK2 inhibitor is Compound 242. In some embodiments, the selective ROCK2 inhibitor is Compound 243. In some embodiments, the selective ROCK2 inhibitor is Compound 244. In some embodiments, the selective ROCK2 inhibitor is Compound 245. In some embodiments, the selective ROCK2 inhibitor is Compound 246. In some embodiments, the selective ROCK2 inhibitor is Compound 247. In some embodiments, the selective ROCK2 inhibitor is Compound 248. In some embodiments, the selective ROCK2 inhibitor is Compound 249. In some embodiments, the selective ROCK2 inhibitor is Compound 250. In some embodiments, the selective ROCK2 inhibitor is Compound 251. In some embodiments, the selective ROCK2 inhibitor is Compound 252. In some embodiments, the selective ROCK2 inhibitor is Compound 253. In some embodiments, the selective ROCK2 inhibitor is Compound 254. In some embodiments, the selective ROCK2 inhibitor is Compound 255. In some embodiments, the selective ROCK2 inhibitor is Compound 256. In some embodiments, the selective ROCK2 inhibitor is Compound 257. In some embodiments, the selective ROCK2 inhibitor is Compound 258. In some embodiments, the selective ROCK2 inhibitor is Compound 259. In some embodiments, the selective ROCK2 inhibitor is Compound 260. In some embodiments, the selective ROCK2 inhibitor is Compound 261. In some embodiments, the selective ROCK2 inhibitor is Compound 262. In some embodiments, the selective ROCK2 inhibitor is Compound 263. In some embodiments, the selective ROCK2 inhibitor is Compound 264. In some embodiments, the selective ROCK2 inhibitor is Compound 265. In some embodiments, the selective ROCK2 inhibitor is Compound 266. In some embodiments, the selective ROCK2 inhibitor is Compound 267. In some embodiments, the selective ROCK2 inhibitor is Compound 268. In some embodiments, the selective ROCK2 inhibitor is Compound 269. In some embodiments, the selective ROCK2 inhibitor is Compound 270. In some embodiments, the selective ROCK2 inhibitor is Compound 271. In some embodiments, the selective ROCK2 inhibitor is Compound 272. In some embodiments, the selective ROCK2 inhibitor is Compound 273. In some embodiments, the selective ROCK2 inhibitor is Compound 274. In some embodiments, the selective ROCK2 inhibitor is Compound 275. In some embodiments, the selective ROCK2 inhibitor is Compound 276. In some embodiments, the selective ROCK2 inhibitor is Compound 277. In some embodiments, the selective ROCK2 inhibitor is Compound 278. In some embodiments, the selective ROCK2 inhibitor is Compound 279. In some embodiments, the selective ROCK2 inhibitor is Compound 280. In some embodiments, the selective ROCK2 inhibitor is Compound 281. In some embodiments, the selective ROCK2 inhibitor is Compound 282. In some embodiments, the selective ROCK2 inhibitor is Compound 283. In some embodiments, the selective ROCK2 inhibitor is Compound 284. In some embodiments, the selective ROCK2 inhibitor is Compound 285. In some embodiments, the selective ROCK2 inhibitor is Compound 286. In some embodiments, the selective ROCK2 inhibitor is Compound 287. In some embodiments, the selective ROCK2 inhibitor is Compound 288. In some embodiments, the selective ROCK2 inhibitor is Compound 289. In some embodiments, the selective ROCK2 inhibitor is Compound 290. In some embodiments, the selective ROCK2 inhibitor is Compound 291. In some embodiments, the selective ROCK2 inhibitor is Compound 292. In some embodiments, the selective ROCK2 inhibitor is Compound 293. In some embodiments, the selective ROCK2 inhibitor is Compound 294. In some embodiments, the selective ROCK2 inhibitor is Compound 295. In some embodiments, the selective ROCK2 inhibitor is Compound 296. In some embodiments, the selective ROCK2 inhibitor is Compound 297. In some embodiments, the selective ROCK2 inhibitor is Compound 298. In some embodiments, the selective ROCK2 inhibitor is Compound 299. In some embodiments, the selective ROCK2 inhibitor is Compound 300. In some embodiments, the selective ROCK2 inhibitor is Compound 301. In some embodiments, the selective ROCK2 inhibitor is Compound 302. In some embodiments, the selective ROCK2 inhibitor is Compound 303. In some embodiments, the selective ROCK2 inhibitor is Compound 304. In some embodiments, the selective ROCK2 inhibitor is Compound 305. In some embodiments, the selective ROCK2 inhibitor is Compound 306. In some embodiments, the selective ROCK2 inhibitor is Compound 307. In some embodiments, the selective ROCK2 inhibitor is Compound 308. In some embodiments, the selective ROCK2 inhibitor is Compound 309. In some embodiments, the selective ROCK2 inhibitor is Compound 310. In some embodiments, the selective ROCK2 inhibitor is Compound 311. In some embodiments, the selective ROCK2 inhibitor is Compound 312. In some embodiments, the selective ROCK2 inhibitor is Compound 313. In some embodiments, the selective ROCK2 inhibitor is Compound 314. In some embodiments, the selective ROCK2 inhibitor is Compound 315. In some embodiments, the selective ROCK2 inhibitor is Compound 316. In some embodiments, the selective ROCK2 inhibitor is Compound 317.

In some embodiments, the selective ROCK2 small molecule inhibitor is a pharmaceutically acceptable salt, stereoisomer, tautomer, isotopically labeled derivative, solvate, hydrate, polymorph, co-crystal, or prodrug of any one of the foregoing compounds (e.g., Compounds 1-317 of Table 1). In some embodiments, the selective ROCK2 small molecule inhibitor is a pharmaceutically acceptable salt of any one of the foregoing compounds (e.g., Compounds 1-317 of Table 1). In some embodiments, the selective ROCK2 small molecule inhibitor is a stereoisomer of any one of the foregoing compounds (e.g., Compounds 1-317 of Table 1). In some embodiments, the selective ROCK2 small molecule inhibitor is a tautomer of any one of the foregoing compounds (e.g., Compounds 1-317 of Table 1). In some embodiments, the selective ROCK2 small molecule inhibitor is an isotopically labeled derivative (e.g., deuterated analog) of any one of the foregoing compounds (e.g., Compounds 1-317 of Table 1). In some embodiments, the selective ROCK2 small molecule inhibitor is a solvate of any one of the foregoing compounds (e.g., Compounds 1-317 of Table 1). In some embodiments, the selective ROCK2 small molecule inhibitor is a hydrate of any one of the foregoing compounds (e.g., Compounds 1-317 of Table 1). In some embodiments, the selective ROCK2 small molecule inhibitor is a polymorph of any one of the foregoing compounds (e.g., Compounds 1-317 of Table 1). In some embodiments, the selective ROCK2 small molecule inhibitor is a co-crystal of any one of the foregoing compounds (e.g., Compounds 1-317 of Table 1). In some embodiments, the selective ROCK2 small molecule inhibitor is a prodrug of any one of the foregoing compounds (e.g., Compounds 1-317 of Table 1).

As used herein, the term “salt” refers to any and all salts and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Other non-limiting examples of selective ROCK2 inhibitors are provided in Table 2 and in International Publication Number WO 2016/138335 A1, incorporated by reference herein in its entirety. In some embodiments, the selective ROCK2 small molecule inhibitor is a compound of Formula (I) or (II) (i.e., including any subgenera or species thereof) disclosed in International Publication Number WO 2016/138335 A1, incorporated by reference herein in its entirety; or a pharmaceutically acceptable salt, stereoisomer, tautomer, isotopically labeled derivative, solvate, hydrate, polymorph, co-crystal, or prodrug thereof. In some embodiments, the selective ROCK2 small molecule inhibitor is a compound of Formula (I) or (II) (i.e., including any subgenera or species thereof) disclosed in International Publication Number WO 2016/138335 A1, incorporated by reference herein in its entirety; or a pharmaceutically acceptable salt thereof.

In some embodiments, the selective ROCK2 small molecule inhibitor comprises a compound selected from Compounds 318-400 of Table 2. In some embodiments, the selective ROCK2 small molecule inhibitor is selected from Compounds 318-400 of Table 2. In some embodiments, the selective ROCK2 small molecule inhibitor is any one of Compounds 318-400 of Table 2, or a pharmaceutically acceptable salt, stereoisomer, tautomer, isotopically labeled derivative, solvate, hydrate, polymorph, co-crystal, or prodrug thereof. In some embodiments, the selective ROCK2 small molecule inhibitor is any one of Compounds 318-400 of Table 2, or a pharmaceutically acceptable salt thereof. In instances where a particular salt form (e.g., trifluoroacetic acid (TFA) salt) of a compound is shown in Table 2, other pharmaceutically acceptable salts of the compound are also provided as part of the present disclosure.

TABLE 2
Examples of Selective ROCK2 Inhibitors

| # | Structure |
|---|---|
| 318 | 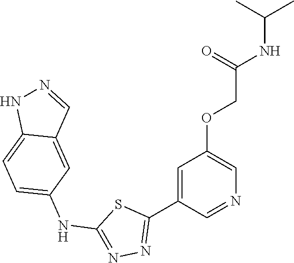 [SMILES: CC(C)NC(=O)COc1cncc(-c2nnc(Nc3ccc4[nH]ncc4c3)s2)c1] |
| 319 | 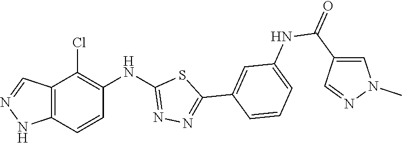 [SMILES: Cn1cc(C(=O)Nc2cccc(-c3nnc(Nc4ccc5[nH]ncc5c4Cl)s3)c2)cn1] |
| 320 | 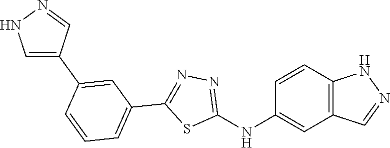 [SMILES: c1cc(-c2cn[nH]c2)cc(-c2nnc(Nc3ccc4[nH]ncc4c3)s2)c1] |
| 321 | 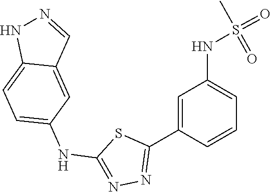 [SMILES: CS(=O)(=O)Nc1cccc(-c2nnc(Nc3ccc4[nH]ncc4c3)s2)c1] |
| 322 | 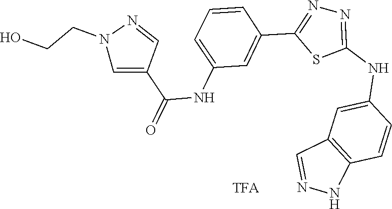 [SMILES: O=C(Nc1cccc(-c2nnc(Nc3ccc4[nH]ncc4c3)s2)c1)c1cnn(CCO)c1] |
| 323 | 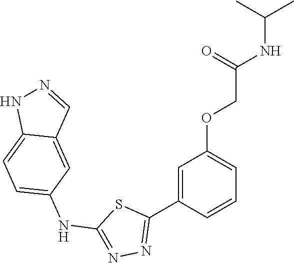 [SMILES: CC(C)NC(=O)COc1cccc(-c2nnc(Nc3ccc4[nH]ncc4c3)s2)c1] |
| 324 | 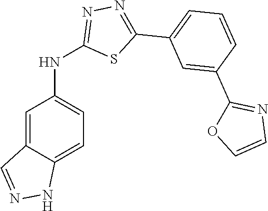 [SMILES: c1cc(-c2ncco2)cc(-c2nnc(Nc3ccc4[nH]ncc4c3)s2)c1] |
| 325 | 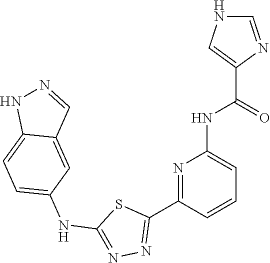 [SMILES: O=C(Nc1cccc(-c2nnc(Nc3ccc4[nH]ncc4c3)s2)n1)c1c[nH]cn1] |
| 326 | 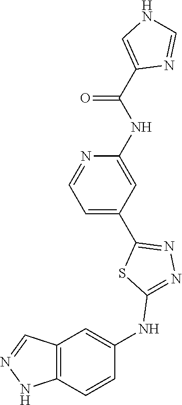 [SMILES: O=C(Nc1cc(-c2nnc(Nc3ccc4[nH]ncc4c3)s2)ccn1)c1c[nH]cn1] |
| 327 | 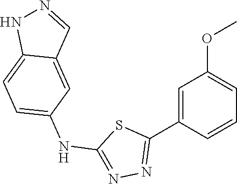 [SMILES: COc1cccc(-c2nnc(Nc3ccc4[nH]ncc4c3)s2)c1] |
| 328 | 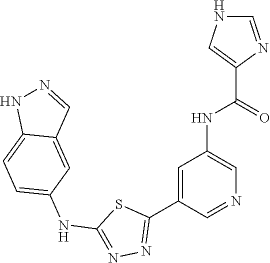 [SMILES: O=C(Nc1cncc(-c2nnc(Nc3ccc4[nH]ncc4c3)s2)c1)c1c[nH]cn1] |
| 329 | 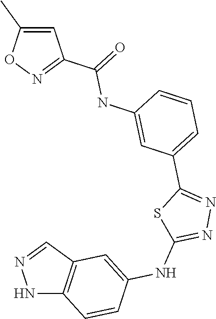 [SMILES: Cc1cc(C(=O)Nc2cccc(-c3nnc(Nc4ccc5[nH]ncc5c4)s3)c2)no1] |
| 330 | 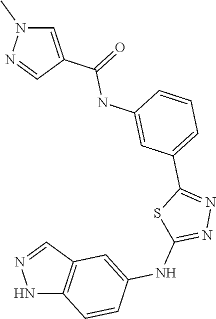 [SMILES: Cn1cc(C(=O)Nc2cccc(-c3nnc(Nc4ccc5[nH]ncc5c4)s3)c2)cn1] |
| 331 | 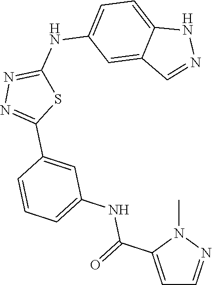 [SMILES: Cn1nccc1C(=O)Nc1cccc(-c2nnc(Nc3ccc4[nH]ncc4c3)s2)c1] |
| 332 | 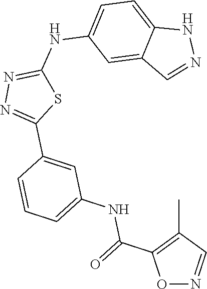 [SMILES: Cc1cnoc1C(=O)Nc1cccc(-c2nnc(Nc3ccc4[nH]ncc4c3)s2)c1] |
| 333 | 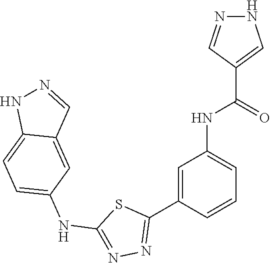 [SMILES: O=C(Nc1cccc(-c2nnc(Nc3ccc4[nH]ncc4c3)s2)c1)c1cn[nH]c1] |
| 334 | 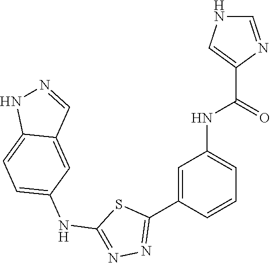 [SMILES: O=C(Nc1cccc(-c2nnc(Nc3ccc4[nH]ncc4c3)s2)c1)c1c[nH]cn1] |
| 335 | 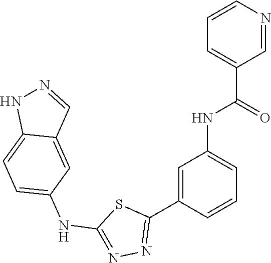 [SMILES: O=C(Nc1cccc(-c2nnc(Nc3ccc4[nH]ncc4c3)s2)c1)c1cccnc1] |
| 336 | 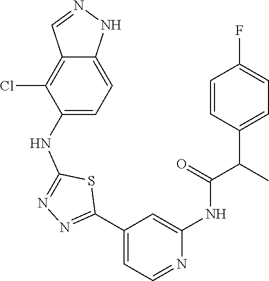 [SMILES: CC(C(=O)Nc1cc(-c2nnc(Nc3ccc4[nH]ncc4c3Cl)s2)ccn1)c1ccc(F)cc1] |
| 337 | 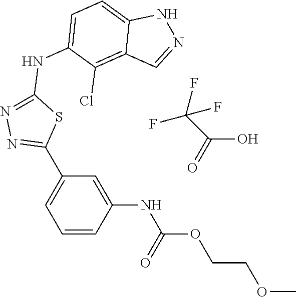 |
| 338 | 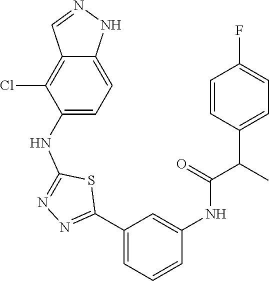 [SMILES: CC(C(=O)Nc1cccc(-c2nnc(Nc3ccc4[nH]ncc4c3Cl)s2)c1)c1ccc(F)cc1] |
| 339 | 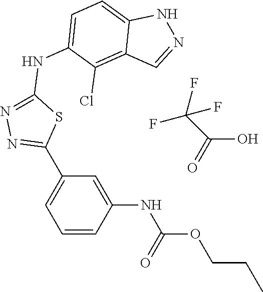 |
| 340 | 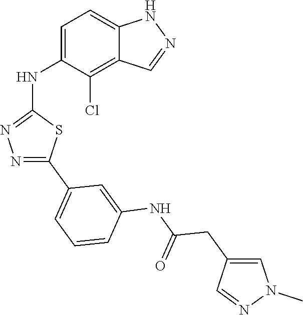 [SMILES: Cn1cc(CC(=O)Nc2cccc(-c3nnc(Nc4ccc5[nH]ncc5c4Cl)s3)c2)cn1] |
| 341 | 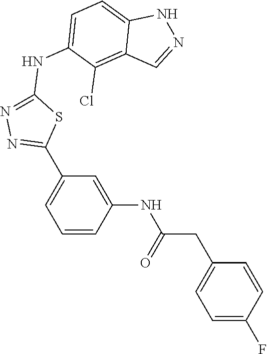 [SMILES: O=C(Cc1ccc(F)cc1)Nc1cccc(-c2nnc(Nc3ccc4[nH]ncc4c3Cl)s2)c1] |
| 342 | 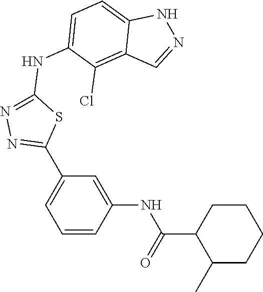 [SMILES: CC1CCCCC1C(=O)Nc1cccc(-c2nnc(Nc3ccc4[nH]ncc4c3Cl)s2)c1] |
| 343 | 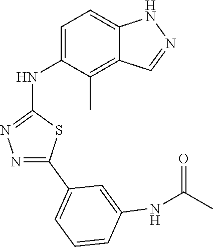 [SMILES: CC(=O)Nc1cccc(-c2nnc(Nc3ccc4[nH]ncc4c3C)s2)c1] |
| 344 | 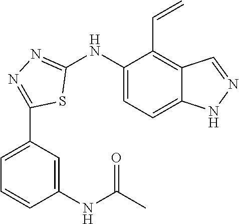 [SMILES: C=Cc1c(Nc2nnc(-c3cccc(NC(C)=O)c3)s2)ccc2[nH]ncc12] |
| 345 | 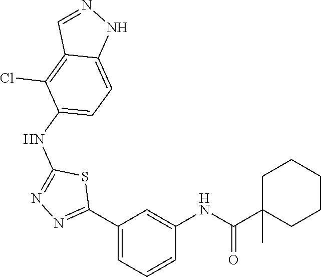 [SMILES: CC1(C(=O)Nc2cccc(-c3nnc(Nc4ccc5[nH]ncc5c4Cl)s3)c2)CCCCC1] |
| 346 | 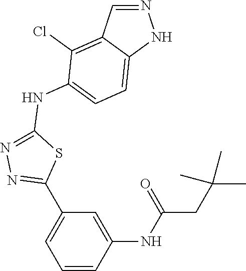 [SMILES: CC(C)(C)CC(=O)Nc1cccc(-c2nnc(Nc3ccc4[nH]ncc4c3Cl)s2)c1] |
| 347 | 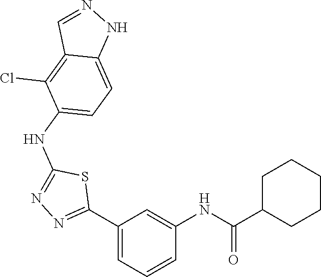 [SMILES: O=C(Nc1cccc(-c2nnc(Nc3ccc4[nH]ncc4c3Cl)s2)c1)C1CCCCC1] |
| 348 | 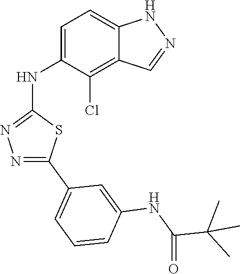 [SMILES: CC(C)(C)C(=O)Nc1cccc(-c2nnc(Nc3ccc4[nH]ncc4c3Cl)s2)c1] |
| 349 | 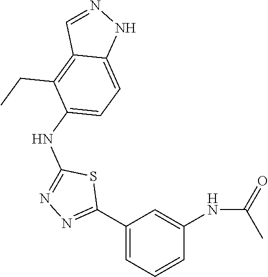 [SMILES: CCc1c(Nc2nnc(-c3cccc(NC(C)=O)c3)s2)ccc2[nH]ncc12] |
| 350 | 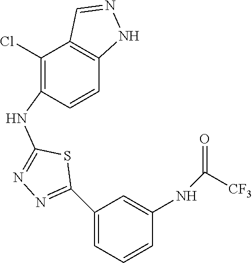 [SMILES: O=C(Nc1cccc(-c2nnc(Nc3ccc4[nH]ncc4c3Cl)s2)c1)C(F)(F)F] |
| 351 | 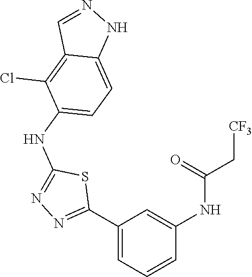 [SMILES: O=C(CC(F)(F)F)Nc1cccc(-c2nnc(Nc3ccc4[nH]ncc4c3Cl)s2)c1] |
| 352 | 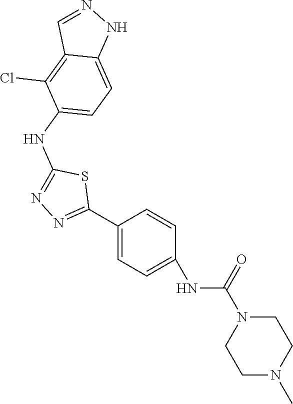 [SMILES: CN1CCN(C(=O)Nc2ccc(-c3nnc(Nc4ccc5[nH]ncc5c4Cl)s3)cc2)CC1] |
| 353 | 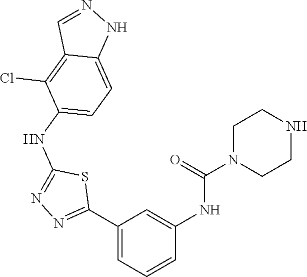 [SMILES: O=C(Nc1cccc(-c2nnc(Nc3ccc4[nH]ncc4c3Cl)s2)c1)N1CCNCC1] |
| 354 | 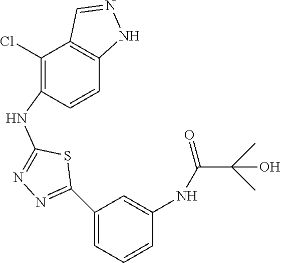 [SMILES: CC(C)(O)C(=O)Nc1cccc(-c2nnc(Nc3ccc4[nH]ncc4c3Cl)s2)c1] |
| 355 | 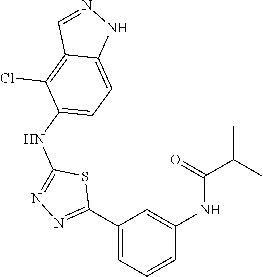 [SMILES: CC(C)C(=O)Nc1cccc(-c2nnc(Nc3ccc4[nH]ncc4c3Cl)s2)c1] |
| 356 | 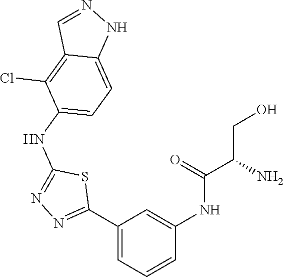 [SMILES: N[C@@H](CO)C(=O)Nc1cccc(-c2nnc(Nc3ccc4[nH]ncc4c3Cl)s2)c1] |
| 357 | 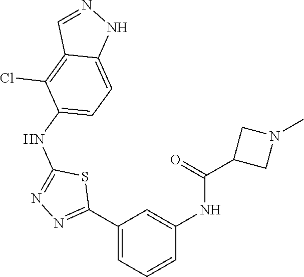 [SMILES: CN1CC(C(=O)Nc2cccc(-c3nnc(Nc4ccc5[nH]ncc5c4Cl)s3)c2)C1] |
| 358 | 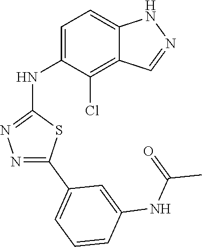 [SMILES: CC(=O)Nc1cccc(-c2nnc(Nc3ccc4[nH]ncc4c3Cl)s2)c1] |
| 359 | 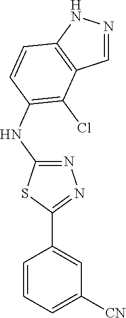 [SMILES: N#Cc1cccc(-c2nnc(Nc3ccc4[nH]ncc4c3Cl)s2)c1] |
| 360 | 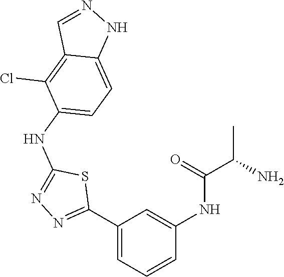 [SMILES: C[C@H](N)C(=O)Nc1cccc(-c2nnc(Nc3ccc4[nH]ncc4c3Cl)s2)c1] |
| 361 | 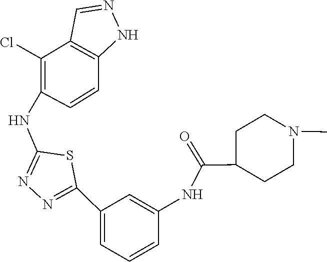 [SMILES: CN1CCC(C(=O)Nc2cccc(-c3nnc(Nc4ccc5[nH]ncc5c4Cl)s3)c2)CC1] |
| 362 | 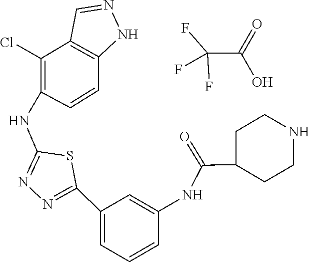 |
| 363 | 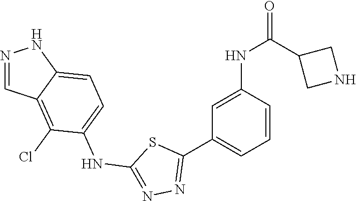 [SMILES: O=C(Nc1cccc(-c2nnc(Nc3ccc4[nH]ncc4c3Cl)s2)c1)C1CNC1] |
| 364 | 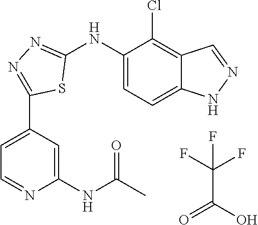 |
| 365 | 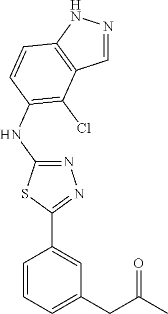 [SMILES: CC(=O)Cc1cccc(-c2nnc(Nc3ccc4[nH]ncc4c3Cl)s2)c1] |
| 366 | 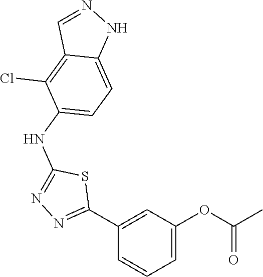 [SMILES: CC(=O)Oc1cccc(-c2nnc(Nc3ccc4[nH]ncc4c3Cl)s2)c1] |
| 367 | 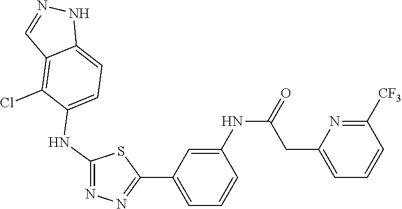 [SMILES: O=C(Cc1cccc(C(F)(F)F)n1)Nc1cccc(-c2nnc(Nc3ccc4[nH]ncc4c3Cl)s2)c1] |
| 368 | 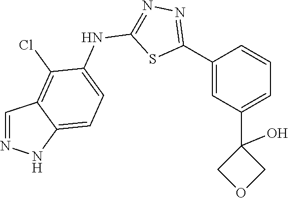 [SMILES: OC1(c2cccc(-c3nnc(Nc4ccc5[nH]ncc5c4Cl)s3)c2)COC1] |
| 369 | 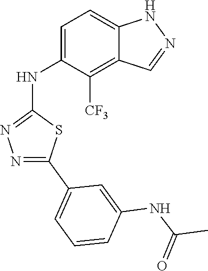 [SMILES: CC(=O)Nc1cccc(-c2nnc(Nc3ccc4[nH]ncc4c3C(F)(F)F)s2)c1] |
| 370 | 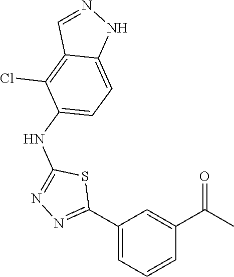 [SMILES: CC(=O)c1cccc(-c2nnc(Nc3ccc4[nH]ncc4c3Cl)s2)c1] |
| 371 | 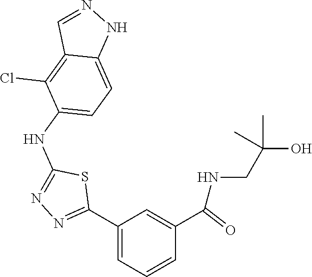 [SMILES: CC(C)(O)CNC(=O)c1cccc(-c2nnc(Nc3ccc4[nH]ncc4c3Cl)s2)c1] |
| 372 | 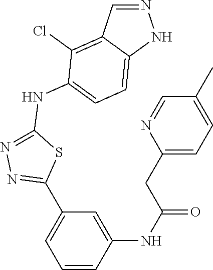 [SMILES: Cc1ccc(CC(=O)Nc2cccc(-c3nnc(Nc4ccc5[nH]ncc5c4Cl)s3)c2)nc1] |
| 373 | 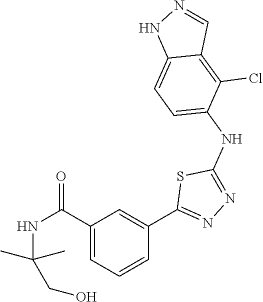 [SMILES: CC(C)(CO)NC(=O)c1cccc(-c2nnc(Nc3ccc4[nH]ncc4c3Cl)s2)c1] |
| 374 | 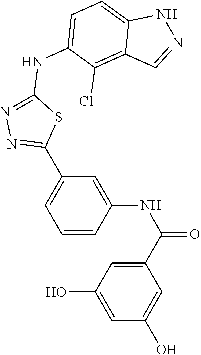 [SMILES: O=C(Nc1cccc(-c2nnc(Nc3ccc4[nH]ncc4c3Cl)s2)c1)c1cc(O)cc(O)c1] |
| 375 | 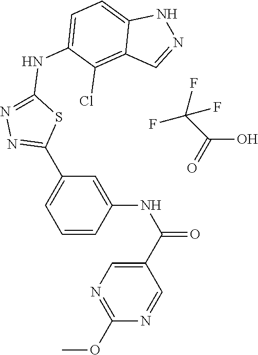 |
| 376 | 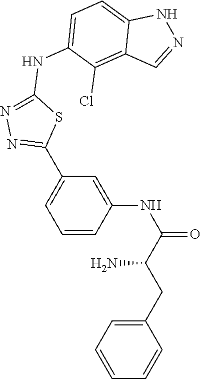 [SMILES: N[C@@H](Cc1ccccc1)C(=O)Nc1cccc(-c2nnc(Nc3ccc4[nH]ncc4c3Cl)s2)c1] |
| 377 | 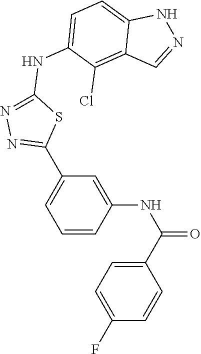 [SMILES: O=C(Nc1cccc(-c2nnc(Nc3ccc4[nH]ncc4c3Cl)s2)c1)c1ccc(F)cc1] |
| 378 | 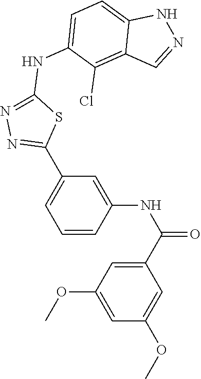 [SMILES: COc1cc(OC)cc(C(=O)Nc2cccc(-c3nnc(Nc4ccc5[nH]ncc5c4Cl)s3)c2)c1] |
| 379 | 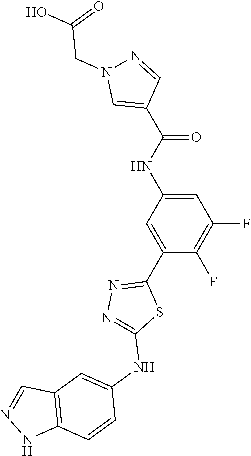 [SMILES: O=C(O)Cn1cc(C(=O)Nc2cc(F)c(F)c(-c3nnc(Nc4ccc5[nH]ncc5c4)s3)c2)cn1] |
| 380 | 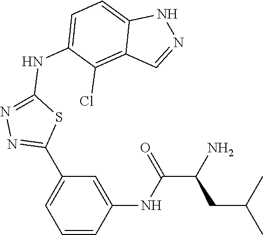 [SMILES: CC(C)C[C@H](N)C(=O)Nc1cccc(-c2nnc(Nc3ccc4[nH]ncc4c3Cl)s2)c1] |
| 381 | 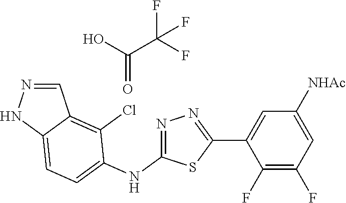 |
| 382 | 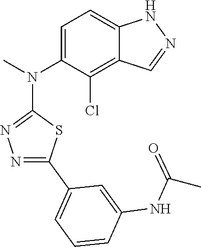 [SMILES: CC(=O)Nc1cccc(-c2nnc(N(C)c3ccc4[nH]ncc4c3Cl)s2)c1] |
| 383 | 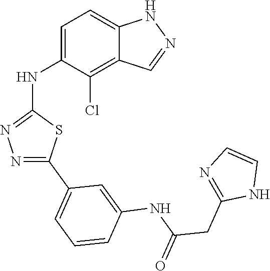 [SMILES: O=C(Cc1ncc[nH]1)Nc1cccc(-c2nnc(Nc3ccc4[nH]ncc4c3Cl)s2)c1] |
| 384 | 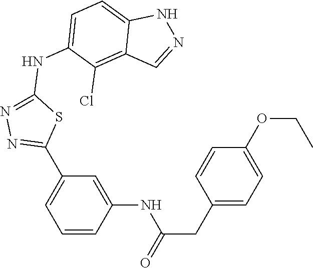 [SMILES: CCOc1ccc(CC(=O)Nc2cccc(-c3nnc(Nc4ccc5[nH]ncc5c4Cl)s3)c2)cc1] |
| 385 | 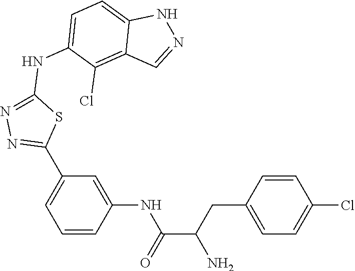 [SMILES: NC(Cc1ccc(Cl)cc1)C(=O)Nc1cccc(-c2nnc(Nc3ccc4[nH]ncc4c3Cl)s2)c1] |
| 386 | 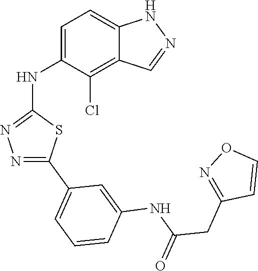 [SMILES: O=C(Cc1ccon1)Nc1cccc(-c2nnc(Nc3ccc4[nH]ncc4c3Cl)s2)c1] |
| 387 | 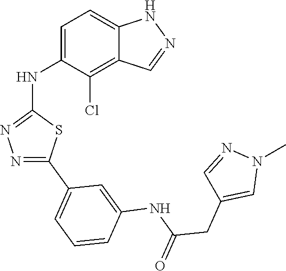 [SMILES: Cn1cc(CC(=O)Nc2cccc(-c3nnc(Nc4ccc5[nH]ncc5c4Cl)s3)c2)cn1] |
| 388 | 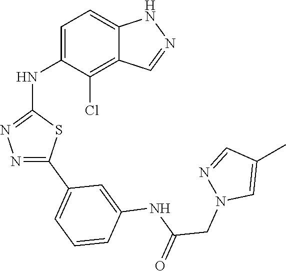 [SMILES: Cc1cnn(CC(=O)Nc2cccc(-c3nnc(Nc4ccc5[nH]ncc5c4Cl)s3)c2)c1] |
| 389 | 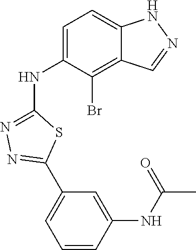 [SMILES: CC(=O)Nc1cccc(-c2nnc(Nc3ccc4[nH]ncc4c3Br)s2)c1] |
| 390 | 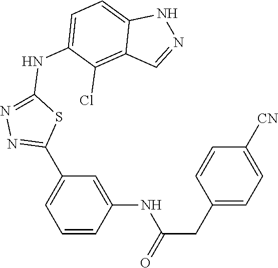 [SMILES: N#Cc1ccc(CC(=O)Nc2cccc(-c3nnc(Nc4ccc5[nH]ncc5c4Cl)s3)c2)cc1] |
| 391 | 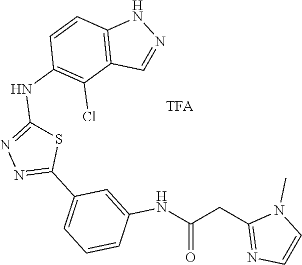 [SMILES: Cn1ccnc1CC(=O)Nc1cccc(-c2nnc(Nc3ccc4[nH]ncc4c3Cl)s2)c1] |
| 392 | 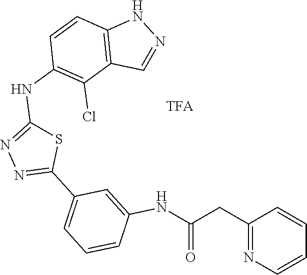 [SMILES: O=C(Cc1ccccn1)Nc1cccc(-c2nnc(Nc3ccc4[nH]ncc4c3Cl)s2)c1] |
| 393 | 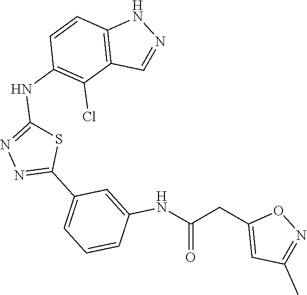 [SMILES: Cc1cc(CC(=O)Nc2cccc(-c3nnc(Nc4ccc5[nH]ncc5c4Cl)s3)c2)on1] |
| 394 | 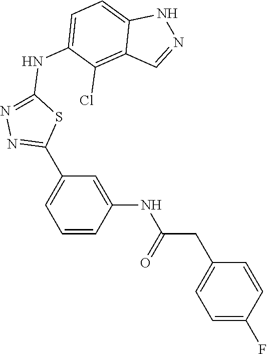 [SMILES: O=C(Cc1ccc(F)cc1)Nc1cccc(-c2nnc(Nc3ccc4[nH]ncc4c3Cl)s2)c1] |
| 395 | 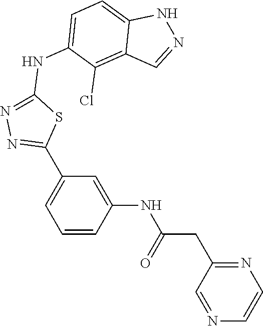 [SMILES: O=C(Cc1cnccn1)Nc1cccc(-c2nnc(Nc3ccc4[nH]ncc4c3Cl)s2)c1] |
| 396 | 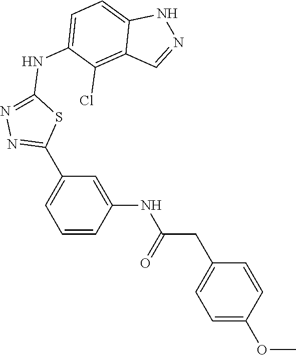 [SMILES: COc1ccc(CC(=O)Nc2cccc(-c3nnc(Nc4ccc5[nH]ncc5c4Cl)s3)c2)cc1] |
| 397 | 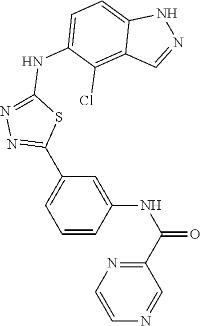 [SMILES: O=C(Nc1cccc(-c2nnc(Nc3ccc4[nH]ncc4c3Cl)s2)c1)c1cnccn1] |
| 398 | 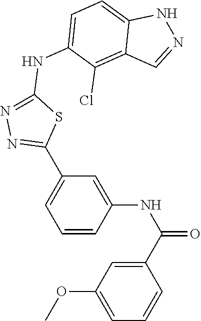 [SMILES: COc1cccc(C(=O)Nc2cccc(-c3nnc(Nc4ccc5[nH]ncc5c4Cl)s3)c2)c1] |
| 399 | 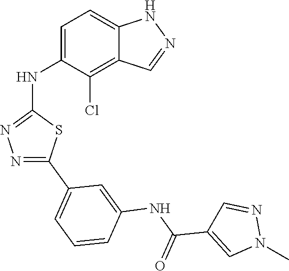 [SMILES: Cn1cc(C(=O)Nc2cccc(-c3nnc(Nc4ccc5[nH]ncc5c4Cl)s3)c2)cn1] |
| 400 | 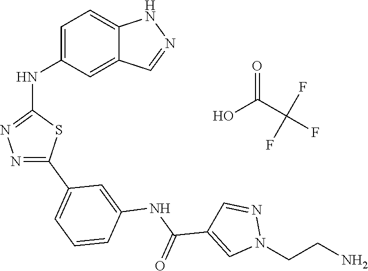 [SMILES: NCCn1cc(C(=O)Nc2cccc(-c3nnc(Nc4ccc5[nH]ncc5c4)s3)c2)cn1 ; O=C(O)C(F)(F)F] |

In some embodiments, the selective ROCK2 inhibitor comprises Compound 318. In some embodiments, the selective ROCK2 inhibitor comprises Compound 319. In some embodiments, the selective ROCK2 inhibitor comprises Compound 320. In some embodiments, the selective ROCK2 inhibitor comprises Compound 321. In some embodiments, the selective ROCK2 inhibitor comprises Compound 322. In some embodiments, the selective ROCK2 inhibitor comprises Compound 323. In some embodiments, the selective ROCK2 inhibitor comprises Compound 324. In some embodiments, the selective ROCK2 inhibitor comprises Compound 325. In some embodiments, the selective ROCK2 inhibitor comprises Compound 326. In some embodiments, the selective ROCK2 inhibitor comprises Compound 327. In some embodiments, the selective ROCK2 inhibitor comprises Compound 328. In some embodiments, the selective ROCK2 inhibitor comprises Compound 329. In some embodiments, the selective ROCK2 inhibitor comprises Compound 330. In some embodiments, the selective ROCK2 inhibitor comprises Compound 331. In some embodiments, the selective ROCK2 inhibitor comprises Compound 332. In some embodiments, the selective ROCK2 inhibitor comprises Compound 333. In some embodiments, the selective ROCK2 inhibitor comprises Compound 334. In some embodiments, the selective ROCK2 inhibitor comprises Compound 335. In some embodiments, the selective ROCK2 inhibitor comprises Compound 336. In some embodiments, the selective ROCK2 inhibitor comprises Compound 337. In some embodiments, the selective ROCK2 inhibitor comprises Compound 338. In some embodiments, the selective ROCK2 inhibitor comprises Compound 339. In some embodiments, the selective ROCK2 inhibitor comprises Compound 340. In some embodiments, the selective ROCK2 inhibitor comprises Compound 341. In some embodiments, the selective ROCK2 inhibitor comprises Compound 342. In some embodiments, the selective ROCK2 inhibitor comprises Compound 343. In some embodiments, the selective ROCK2 inhibitor comprises Compound 344. In some embodiments, the selective ROCK2 inhibitor comprises Compound 345. In some embodiments, the selective ROCK2 inhibitor comprises Compound 346. In some embodiments, the selective ROCK2 inhibitor comprises Compound 347. In some embodiments, the selective ROCK2 inhibitor comprises Compound 348. In some embodiments, the selective ROCK2 inhibitor comprises Compound 349. In some embodiments, the selective ROCK2 inhibitor comprises Compound 350. In some embodiments, the selective ROCK2 inhibitor comprises Compound 351. In some embodiments, the selective ROCK2 inhibitor comprises Compound 352. In some embodiments, the selective ROCK2 inhibitor comprises Compound 353. In some embodiments, the selective ROCK2 inhibitor comprises Compound 354. In some embodiments, the selective ROCK2 inhibitor comprises Compound 355. In some embodiments, the selective ROCK2 inhibitor comprises Compound 356. In some embodiments, the selective ROCK2 inhibitor comprises Compound 357. In some embodiments, the selective ROCK2 inhibitor comprises Compound 358. In some embodiments, the selective ROCK2 inhibitor comprises Compound 359. In some embodiments, the selective ROCK2 inhibitor comprises Compound 360. In some embodiments, the selective ROCK2 inhibitor comprises Compound 361. In some embodiments, the selective ROCK2 inhibitor comprises Compound 362. In some embodiments, the selective ROCK2 inhibitor comprises Compound 363. In some embodiments, the selective ROCK2 inhibitor comprises Compound 364. In some embodiments, the selective ROCK2 inhibitor comprises Compound 365. In some embodiments, the selective ROCK2 inhibitor comprises Compound 366. In some embodiments, the selective ROCK2 inhibitor comprises Compound 367. In some embodiments, the selective ROCK2 inhibitor comprises Compound 368. In some embodiments, the selective ROCK2 inhibitor comprises Compound 369. In some embodiments, the selective ROCK2 inhibitor comprises Compound 370. In some embodiments, the selective ROCK2 inhibitor comprises Compound 371. In some embodiments, the selective ROCK2 inhibitor comprises Compound 372. In some embodiments, the selective ROCK2 inhibitor comprises Compound 373. In some embodiments, the selective ROCK2 inhibitor comprises Compound 374. In some embodiments, the selective ROCK2 inhibitor comprises Compound 375. In some embodiments, the selective ROCK2 inhibitor comprises Compound 376. In some embodiments, the selective ROCK2 inhibitor comprises Compound 377. In some embodiments, the selective ROCK2 inhibitor comprises Compound 378. In some embodiments, the selective ROCK2 inhibitor comprises Compound 379. In some embodiments, the selective ROCK2 inhibitor comprises Compound 380. In some embodiments, the selective ROCK2 inhibitor comprises Compound 381. In some embodiments, the selective ROCK2 inhibitor comprises Compound 382. In some embodiments, the selective ROCK2 inhibitor comprises Compound 383. In some embodiments, the selective ROCK2 inhibitor comprises Compound 384. In some embodiments, the selective ROCK2 inhibitor comprises Compound 385. In some embodiments, the selective ROCK2 inhibitor comprises Compound 386. In some embodiments, the selective ROCK2 inhibitor comprises Compound 387. In some embodiments, the selective ROCK2 inhibitor comprises Compound 388. In some embodiments, the selective ROCK2 inhibitor comprises Compound 389. In some embodiments, the selective ROCK2 inhibitor comprises Compound 390. In some embodiments, the selective ROCK2 inhibitor comprises Compound 391. In some embodiments, the selective ROCK2 inhibitor comprises Compound 392. In some embodiments, the selective ROCK2 inhibitor comprises Compound 393. In some embodiments, the selective ROCK2 inhibitor comprises Compound 394. In some embodiments, the selective ROCK2 inhibitor comprises Compound 395. In some embodiments, the selective ROCK2 inhibitor comprises Compound 396. In some embodiments, the selective ROCK2 inhibitor comprises Compound 397. In some embodiments, the selective ROCK2 inhibitor comprises Compound 398. In some embodiments, the selective ROCK2 inhibitor comprises Compound 399. In some embodiments, the selective ROCK2 inhibitor comprises Compound 400.

In some embodiments, the selective ROCK2 inhibitor is Compound 318. In some embodiments, the selective ROCK2 inhibitor is Compound 319. In some embodiments, the selective ROCK2 inhibitor is Compound 320. In some embodiments, the selective ROCK2 inhibitor is Compound 321. In some embodiments, the selective ROCK2 inhibitor is Compound 322. In some embodiments, the selective ROCK2 inhibitor is Compound 323. In some embodiments, the selective ROCK2 inhibitor is Compound 324. In some embodiments, the selective ROCK2 inhibitor is Compound 325. In some embodiments, the selective ROCK2 inhibitor is Compound 326. In some embodiments, the selective ROCK2 inhibitor is Compound 327. In some embodiments, the selective ROCK2 inhibitor is Compound 328. In some embodiments, the selective ROCK2 inhibitor is Compound 329. In some embodiments, the selective ROCK2 inhibitor is Compound 330. In some embodiments, the selective ROCK2 inhibitor is Compound 331. In some embodiments, the selective ROCK2 inhibitor is Compound 332. In some embodiments, the selective ROCK2 inhibitor is Compound 333. In some embodiments, the selective ROCK2 inhibitor is Compound 334. In some embodiments, the selective ROCK2 inhibitor is Compound 335. In some embodiments, the selective ROCK2 inhibitor is Compound 336. In some embodiments, the selective ROCK2 inhibitor is Compound 337. In some embodiments, the selective ROCK2 inhibitor is Compound 338. In some embodiments, the selective ROCK2 inhibitor is Compound 339. In some embodiments, the selective ROCK2 inhibitor is Compound 340. In some embodiments, the selective ROCK2 inhibitor is Compound 341. In some embodiments, the selective ROCK2 inhibitor is Compound 342. In some embodiments, the selective ROCK2 inhibitor is Compound 343. In some embodiments, the selective ROCK2 inhibitor is Compound 344. In some embodiments, the selective ROCK2 inhibitor is Compound 345. In some embodiments, the selective ROCK2 inhibitor is Compound 346. In some embodiments, the selective ROCK2 inhibitor is Compound 347. In some embodiments, the selective ROCK2 inhibitor is Compound 348. In some embodiments, the selective ROCK2 inhibitor is Compound 349. In some embodiments, the selective ROCK2 inhibitor is Compound 350. In some embodiments, the selective ROCK2 inhibitor is Compound 351. In some embodiments, the selective ROCK2 inhibitor is Compound 352. In some embodiments, the selective ROCK2 inhibitor is Compound 353. In some embodiments, the selective ROCK2 inhibitor is Compound 354. In some embodiments, the selective ROCK2 inhibitor is Compound 355. In some embodiments, the selective ROCK2 inhibitor is Compound 356. In some embodiments, the selective ROCK2 inhibitor is Compound 357. In some embodiments, the selective ROCK2 inhibitor is Compound 358. In some embodiments, the selective ROCK2 inhibitor is Compound 359. In some embodiments, the selective ROCK2 inhibitor is Compound 360. In some embodiments, the selective ROCK2 inhibitor is Compound 361. In some embodiments, the selective ROCK2 inhibitor is Compound 362. In some embodiments, the selective ROCK2 inhibitor is Compound 363. In some embodiments, the selective ROCK2 inhibitor is Compound 364. In some embodiments, the selective ROCK2 inhibitor is Compound 365. In some embodiments, the selective ROCK2 inhibitor is Compound 366. In some embodiments, the selective ROCK2 inhibitor is Compound 367. In some embodiments, the selective ROCK2 inhibitor is Compound 368. In some embodiments, the selective ROCK2 inhibitor is Compound 369. In some embodiments, the selective ROCK2 inhibitor is Compound 370. In some embodiments, the selective ROCK2 inhibitor is Compound 371. In some embodiments, the selective ROCK2 inhibitor is Compound 372. In some embodiments, the selective ROCK2 inhibitor is Compound 373. In some embodiments, the selective ROCK2 inhibitor is Compound 374. In some embodiments, the selective ROCK2 inhibitor is Compound 375. In some embodiments, the selective ROCK2 inhibitor is Compound 376. In some embodiments, the selective ROCK2 inhibitor is Compound 377. In some embodiments, the selective ROCK2 inhibitor is Compound 378. In some embodiments, the selective ROCK2 inhibitor is Compound 379. In some embodiments, the selective ROCK2 inhibitor is Compound 380. In some embodiments, the selective ROCK2 inhibitor is Compound 381. In some embodiments, the selective ROCK2 inhibitor is Compound 382. In some embodiments, the selective ROCK2 inhibitor is Compound 383. In some embodiments, the selective ROCK2 inhibitor is Compound 384. In some embodiments, the selective ROCK2 inhibitor is Compound 385. In some embodiments, the selective ROCK2 inhibitor is Compound 386. In some embodiments, the selective ROCK2 inhibitor is Compound 387. In some embodiments, the selective ROCK2 inhibitor is Compound 388. In some embodiments, the selective ROCK2 inhibitor is Compound 389. In some embodiments, the selective ROCK2 inhibitor is Compound 390. In some embodiments, the selective ROCK2 inhibitor is Compound 391. In some embodiments, the selective ROCK2 inhibitor is Compound 392. In some embodiments, the selective ROCK2 inhibitor is Compound 393. In some embodiments, the selective ROCK2 inhibitor is Compound 394. In some embodiments, the selective ROCK2 inhibitor is Compound 395. In some embodiments, the selective ROCK2 inhibitor is Compound 396. In some embodiments, the selective ROCK2 inhibitor is Compound 397. In some embodiments, the selective ROCK2 inhibitor is Compound 398. In some embodiments, the selective ROCK2 inhibitor is Compound 399. In some embodiments, the selective ROCK2 inhibitor is Compound 400.

Other non-limiting examples of selective ROCK2 inhibitors are provided in Table 3 and in International Publication Number WO 2018/039539 A1, incorporated by reference herein in its entirety. In some embodiments, the selective ROCK2 small molecule inhibitor is a compound of Formula (I) (i.e., including any subgenera or species thereof) disclosed in International Publication Number WO 2018/039539 A1, incorporated by reference herein in its entirety; or a pharmaceutically acceptable salt, stereoisomer, tautomer, isotopically labeled derivative, solvate, hydrate, polymorph, co-crystal, or prodrug thereof. In some embodiments, the selective ROCK2 small molecule inhibitor is a compound of Formula (I) (i.e., including any subgenera or species thereof) disclosed in International Publication Number WO 2018/039539 A1, incorporated by reference herein in its entirety; or a pharmaceutically acceptable salt thereof.

In some embodiments, the selective ROCK2 small molecule inhibitor comprises a compound selected from Compounds 401-483 of Table 3. In some embodiments, the selective ROCK2 small molecule inhibitor is selected from Compounds 401-483 of Table 3. In some embodiments, the selective ROCK2 small molecule inhibitor is any one of Compounds 401-483 of Table 3, or a pharmaceutically acceptable salt, stereoisomer, tautomer, isotopically labeled derivative, solvate, hydrate, polymorph, co-crystal, or prodrug thereof. In some embodiments, the selective ROCK2 small molecule inhibitor is any one of Compounds 401-483 of Table 3, or a pharmaceutically acceptable salt thereof. In instances where a particular salt form (e.g., trifluoroacetic acid (TFA) salt) of a compound is shown in Table 3, other pharmaceutically acceptable salts of the compound are also provided as part of the present disclosure.

TABLE 3
Examples of Selective ROCK2 Inhibitors

| # | Structure |
|---|---|
| 401 | 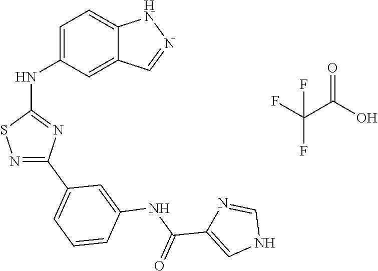 [SMILES: O=C(Nc1cccc(-c2nsc(Nc3ccc4[nH]ncc4c3)n2)c1)c1c[nH]cn1 ; O=C(O)C(F)(F)F] |
| 402 | 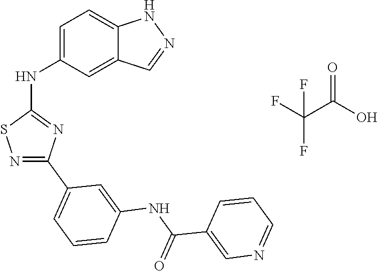 [SMILES: O=C(Nc1cccc(-c2nsc(Nc3ccc4[nH]ncc4c3)n2)c1)c1cccnc1 ; O=C(O)C(F)(F)F] |
| 403 | 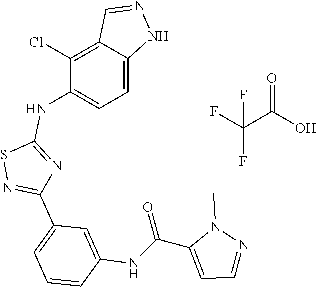 |
| 404 | 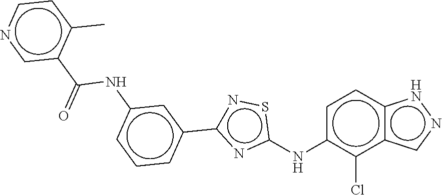 |
| 405 | 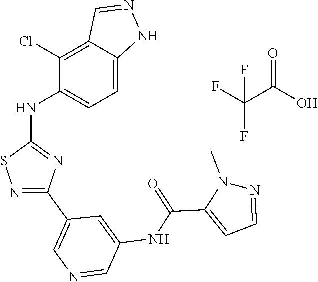 |
| 406 | 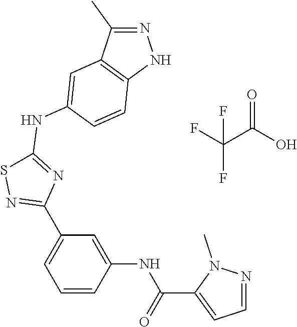 [SMILES: Cc1n[nH]c2ccc(Nc3nc(-c4cccc(NC(=O)c5ccnn5C)c4)ns3)cc12 ; O=C(O)C(F)(F)F] |
| 407 | 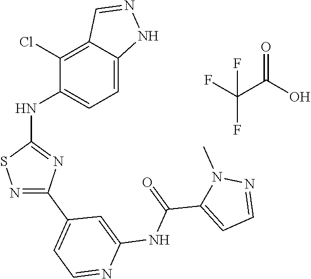 |
| 408 | 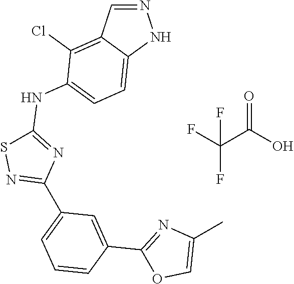 |
| 409 | 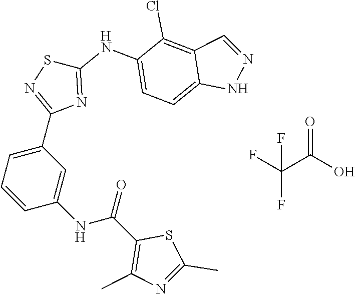 |
| 410 | 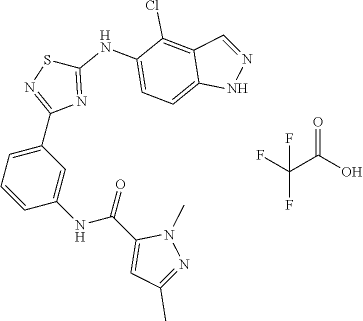 |
| 411 | 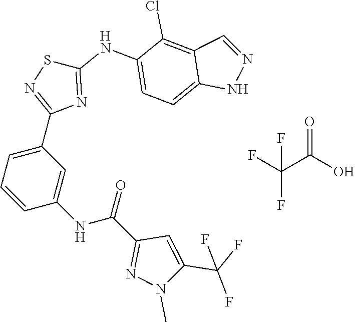 |
| 412 | 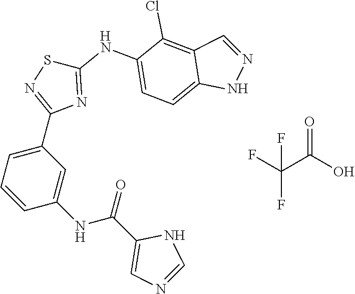 |
| 413 | 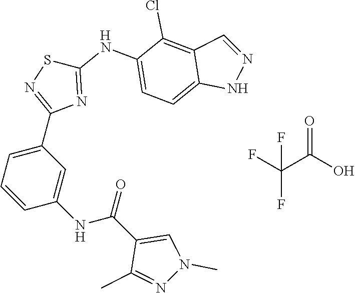 |
| 414 | 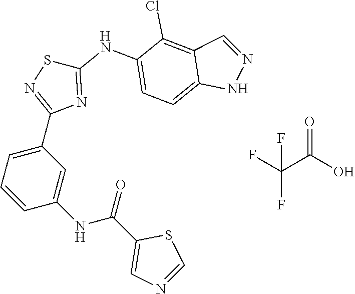 |
| 415 | 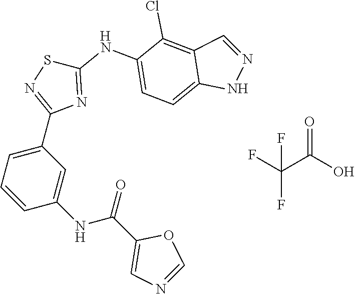 |
| 416 | 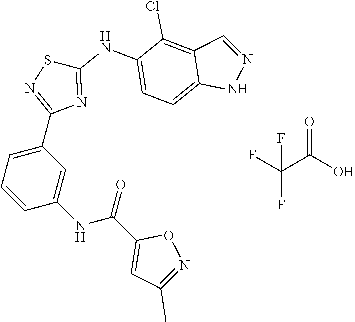 |
| 417 | 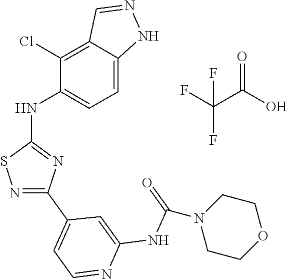 [SMILES: O=C(Nc1cc(-c2nsc(Nc3ccc4[nH]ncc4c3Cl)n2)ccn1)N1CCOCC1 ; O=C(O)C(F)(F)F] |
| 418 | 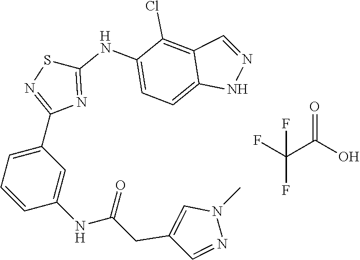 |
| 419 | 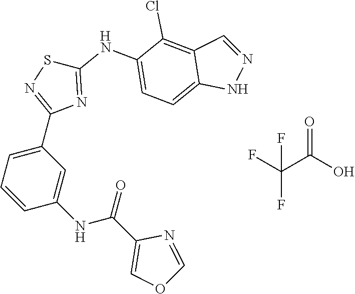 |
| 420 | 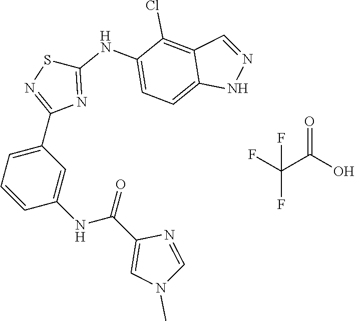 |
| 421 | 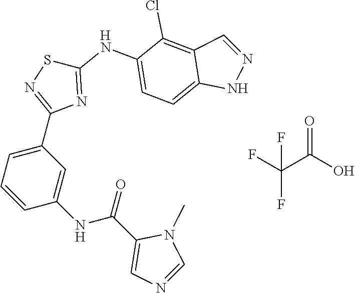 |
| 422 | 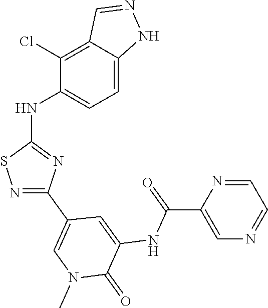 |
| 423 | 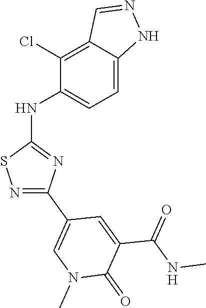 |
| 424 | 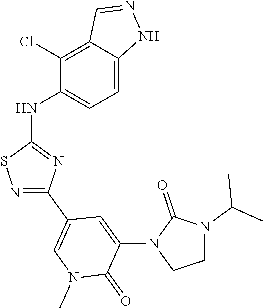 [SMILES: CC(C)N1CCN(c2cc(-c3nsc(Nc4ccc5[nH]ncc5c4Cl)n3)cn(C)c2=O)C1=O] |
| 425 | 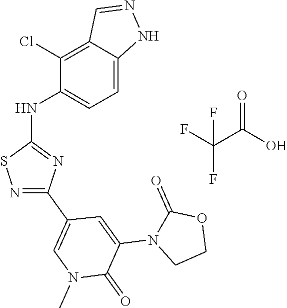 |
| 426 | 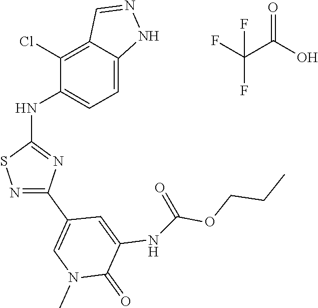 |
| 427 | 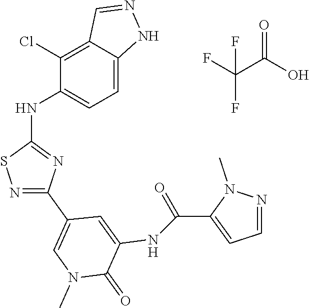 |
| 428 | 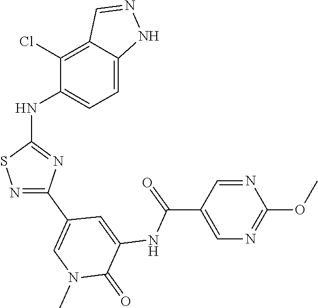 |
| 429 | 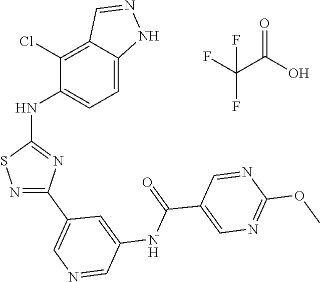 |
| 430 | 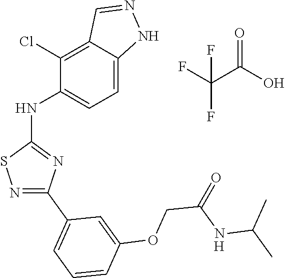 |
| 431 | 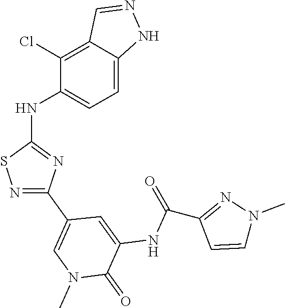 |
| 432 | 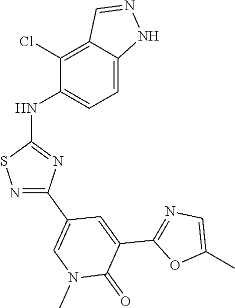 [SMILES: Cc1cnc(-c2cc(-c3nsc(Nc4ccc5[nH]ncc5c4Cl)n3)cn(C)c2=O)o1] |
| 433 | 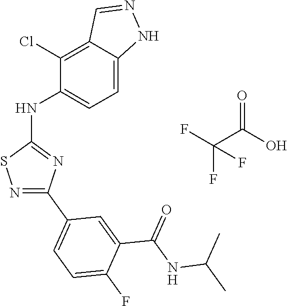 |
| 434 | 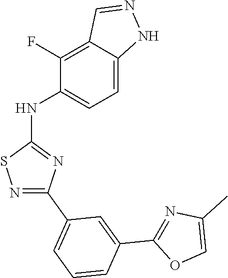 [SMILES: Cc1coc(-c2cccc(-c3nsc(Nc4ccc5[nH]ncc5c4F)n3)c2)n1] |
| 435 | 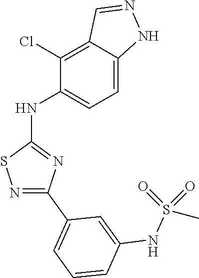 |
| 436 | 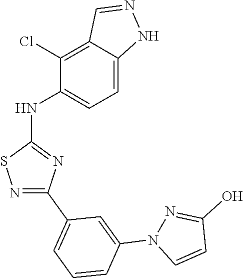 [SMILES: Oc1ccn(-c2cccc(-c3nsc(Nc4ccc5[nH]ncc5c4Cl)n3)c2)n1] |
| 437 | 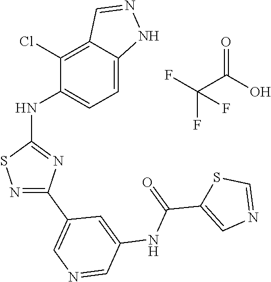 |
| 438 | 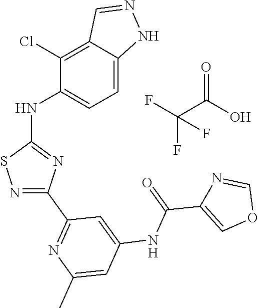 |
| 439 | 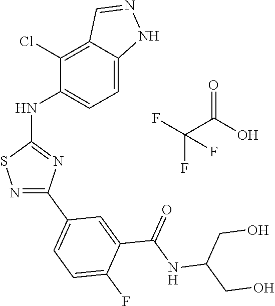 |
| 440 | 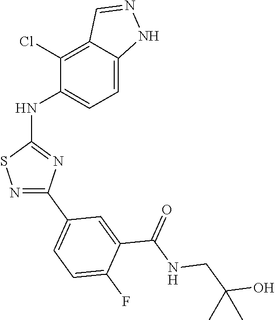 |
| 441 | 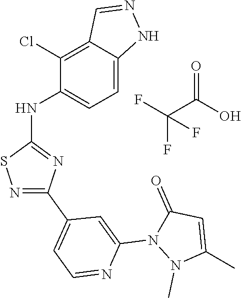 |
| 442 | 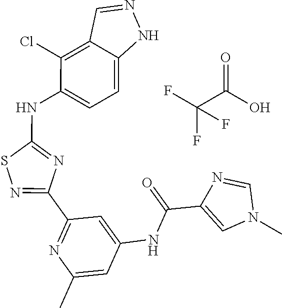 |
| 443 | 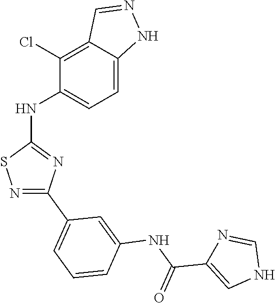 [SMILES: O=C(Nc1cccc(-c2nsc(Nc3ccc4[nH]ncc4c3Cl)n2)c1)c1c[nH]cn1] |
| 444 | 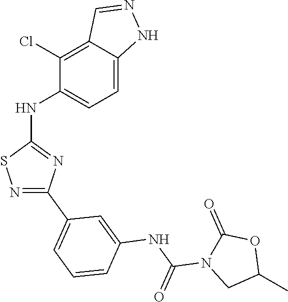 [SMILES: CC1CN(C(=O)Nc2cccc(-c3nsc(Nc4ccc5[nH]ncc5c4Cl)n3)c2)C(=O)O1] |
| 445 | 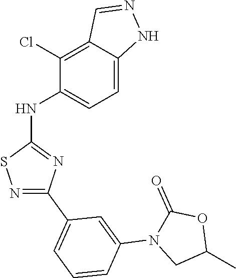 [SMILES: CC1CN(c2cccc(-c3nsc(Nc4ccc5[nH]ncc5c4Cl)n3)c2)C(=O)O1] |
| 446 | 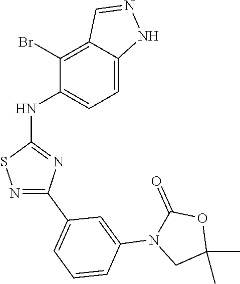 [SMILES: CC1(C)CN(c2cccc(-c3nsc(Nc4ccc5[nH]ncc5c4Br)n3)c2)C(=O)O1] |
| 447 | 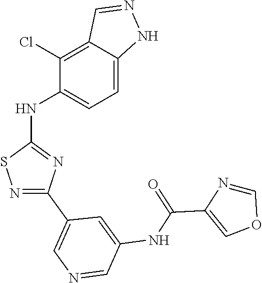 [SMILES: O=C(Nc1cncc(-c2nsc(Nc3ccc4[nH]ncc4c3Cl)n2)c1)c1cocn1] |
| 448 | 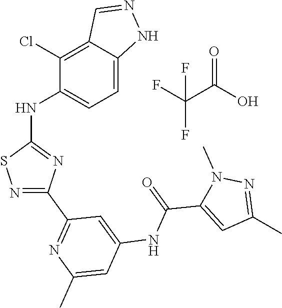 |
| 449 | 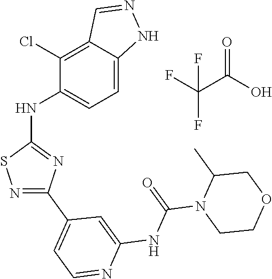 |
| 450 | 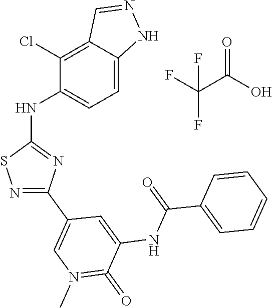 |
| 451 | 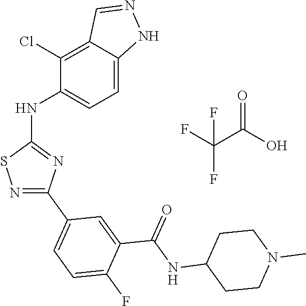 |
| 452 | 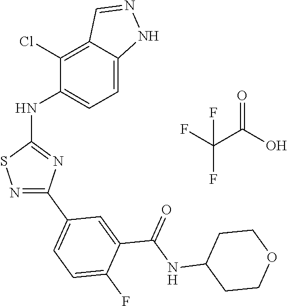 |
| 453 | 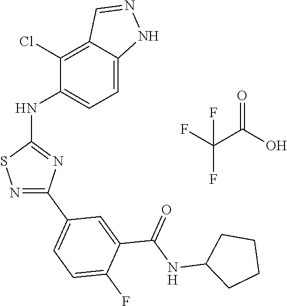 |
| 454 | 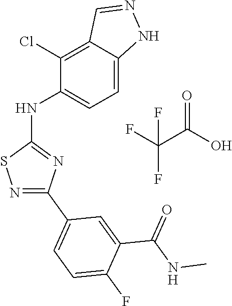 |
| 455 | 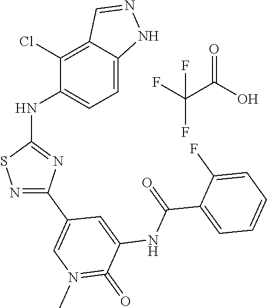 |
| 456 | 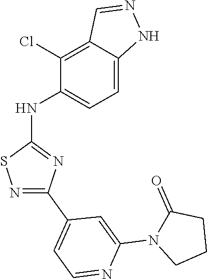 [SMILES: O=C1CCCN1c1cc(-c2nsc(Nc3ccc4[nH]ncc4c3Cl)n2)ccn1] |
| 457 | 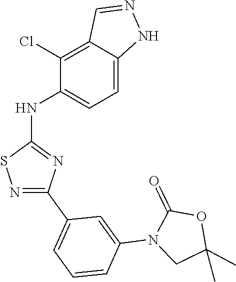 [SMILES: CC1(C)CN(c2cccc(-c3nsc(Nc4ccc5[nH]ncc5c4Cl)n3)c2)C(=O)O1] |
| 458 | 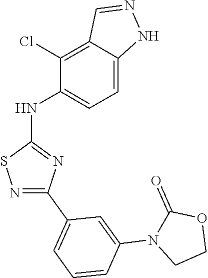 [SMILES: O=C1OCCN1c1cccc(-c2nsc(Nc3ccc4[nH]ncc4c3Cl)n2)c1] |
| 459 | 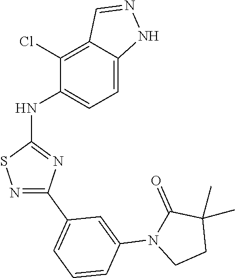 [SMILES: CC1(C)CCN(c2cccc(-c3nsc(Nc4ccc5[nH]ncc5c4Cl)n3)c2)C1=O] |
| 460 | 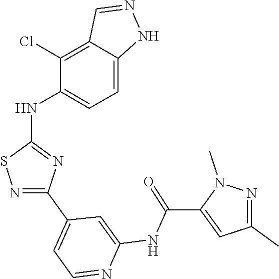 |
| 461 | 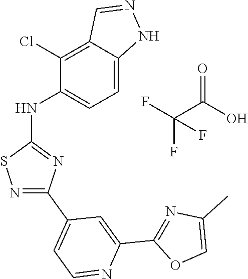 |
| 462 | 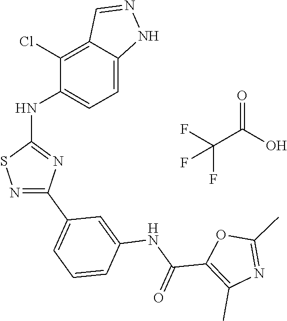 |
| 463 | 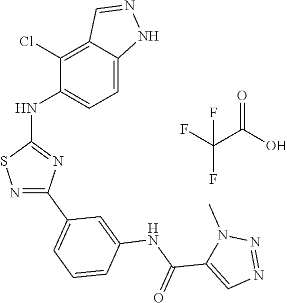 |
| 464 | 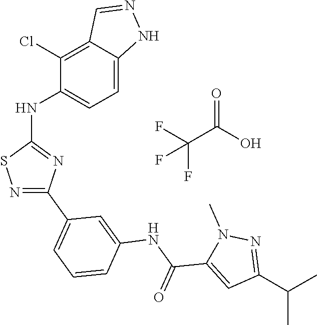 |
| 465 | 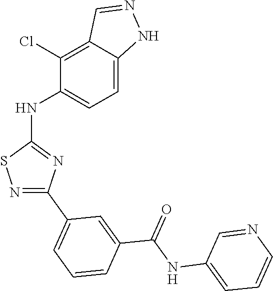 |
| 466 | 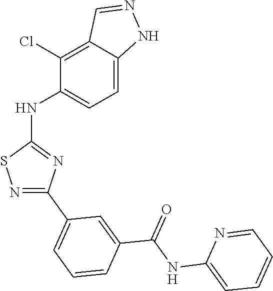 |
| 467 | 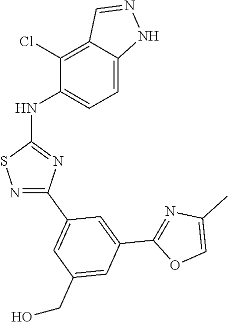 [SMILES: Cc1coc(-c2cc(CO)cc(-c3nsc(Nc4ccc5[nH]ncc5c4Cl)n3)c2)n1] |
| 468 | 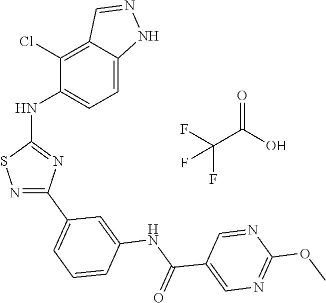 |
| 469 | 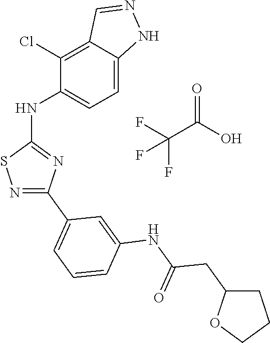 |
| 470 | 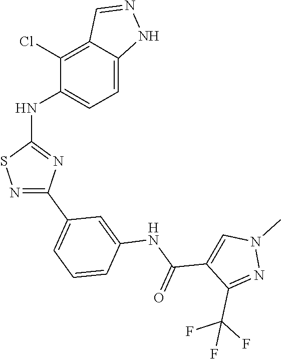 |
| 471 | 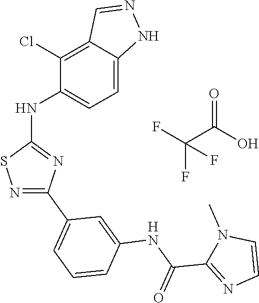 |
| 472 | 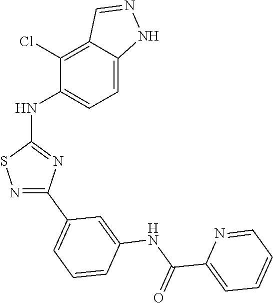 |
| 473 | 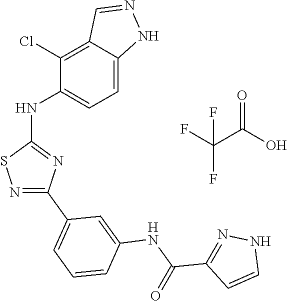 |
| 474 | 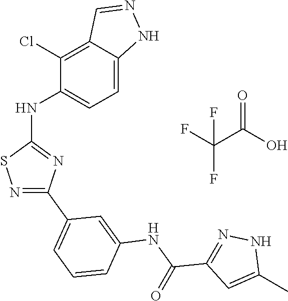 |
| 475 | 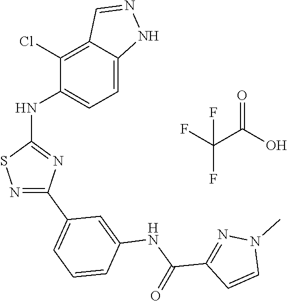 |
| 476 | 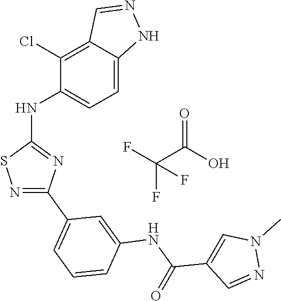 |
| 477 | 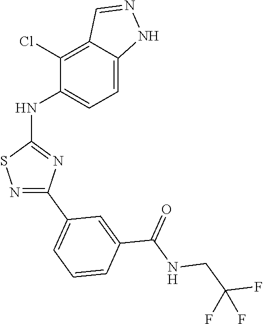 |
| 478 | 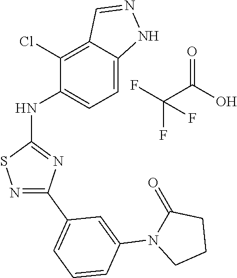 |
| 479 | 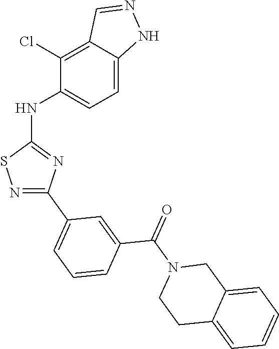 [SMILES: O=C(c1cccc(-c2nsc(Nc3ccc4[nH]ncc4c3Cl)n2)c1)N1CCc2ccccc2C1] |
| 480 | 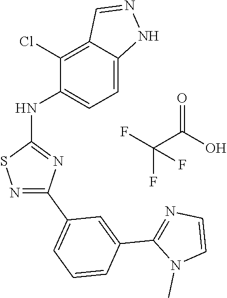 |
| 481 | 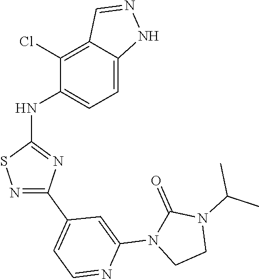 [SMILES: CC(C)N1CCN(c2cc(-c3nsc(Nc4ccc5[nH]ncc5c4Cl)n3)ccn2)C1=O] |
| 482 | 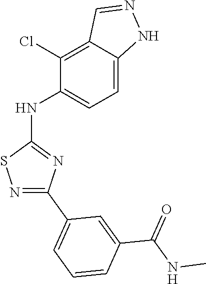 |
| 483 | 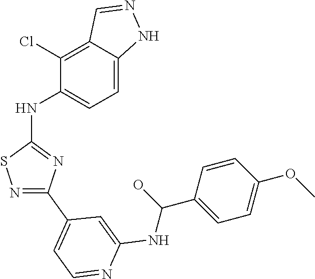 [SMILES: COc1ccc(C(O)Nc2cc(-c3nsc(Nc4ccc5[nH]ncc5c4Cl)n3)ccn2)cc1] |

# = Compound

In some embodiments, the selective ROCK2 inhibitor comprises Compound 401. In some embodiments, the selective ROCK2 inhibitor comprises Compound 402. In some embodiments, the selective ROCK2 inhibitor comprises Compound 403. In some embodiments, the selective ROCK2 inhibitor comprises Compound 404. In some embodiments, the selective ROCK2 inhibitor comprises Compound 405. In some embodiments, the selective ROCK2 inhibitor comprises Compound 406. In some embodiments, the selective ROCK2 inhibitor comprises Compound 407. In some embodiments, the selective ROCK2 inhibitor comprises Compound 408. In some embodiments, the selective ROCK2 inhibitor comprises Compound 409. In some embodiments, the selective ROCK2 inhibitor comprises Compound 410. In some embodiments, the selective ROCK2 inhibitor comprises Compound 411. In some embodiments, the selective ROCK2 inhibitor comprises Compound 412. In some embodiments, the selective ROCK2 inhibitor comprises Compound 413. In some embodiments, the selective ROCK2 inhibitor comprises Compound 414. In some embodiments, the selective ROCK2 inhibitor comprises Compound 415. In some embodiments, the selective ROCK2 inhibitor comprises Compound 416. In some embodiments, the selective ROCK2 inhibitor comprises Compound 417. In some embodiments, the selective ROCK2 inhibitor comprises Compound 418. In some embodiments, the selective ROCK2 inhibitor comprises Compound 419. In some embodiments, the selective ROCK2 inhibitor comprises Compound 420. In some embodiments, the selective ROCK2 inhibitor comprises Compound 421. In some embodiments, the selective ROCK2 inhibitor comprises Compound 422. In some embodiments, the selective ROCK2 inhibitor comprises Compound 423. In some embodiments, the selective ROCK2 inhibitor comprises Compound 424. In some embodiments, the selective ROCK2 inhibitor comprises Compound 425. In some embodiments, the selective ROCK2 inhibitor comprises Compound 426. In some embodiments, the selective ROCK2 inhibitor comprises Compound 427. In some embodiments, the selective ROCK2 inhibitor comprises Compound 428. In some embodiments, the selective ROCK2 inhibitor comprises Compound 429. In some embodiments, the selective ROCK2 inhibitor comprises Compound 430. In some embodiments, the selective ROCK2 inhibitor comprises Compound 431. In some embodiments, the selective ROCK2 inhibitor comprises Compound 432. In some embodiments, the selective ROCK2 inhibitor comprises Compound 433. In some embodiments, the selective ROCK2 inhibitor comprises Compound 434. In some embodiments, the selective ROCK2 inhibitor comprises Compound 435. In some embodiments, the selective ROCK2 inhibitor comprises Compound 436. In some embodiments, the selective ROCK2 inhibitor comprises Compound 437. In some embodiments, the selective ROCK2 inhibitor comprises Compound 438. In some embodiments, the selective ROCK2 inhibitor comprises Compound 439. In some embodiments, the selective ROCK2 inhibitor comprises Compound 440. In some embodiments, the selective ROCK2 inhibitor comprises Compound 441. In some embodiments, the selective ROCK2 inhibitor comprises Compound 442. In some embodiments, the selective ROCK2 inhibitor comprises Compound 443. In some embodiments, the selective ROCK2 inhibitor comprises Compound 444. In some embodiments, the selective ROCK2 inhibitor comprises Compound 445. In some embodiments, the selective ROCK2 inhibitor comprises Compound 446. In some embodiments, the selective ROCK2 inhibitor comprises Compound 447. In some embodiments, the selective ROCK2 inhibitor comprises Compound 448. In some embodiments, the selective ROCK2 inhibitor comprises Compound 449. In some embodiments, the selective ROCK2 inhibitor comprises Compound 450. In some embodiments, the selective ROCK2 inhibitor comprises Compound 451. In some embodiments, the selective ROCK2 inhibitor comprises Compound 452. In some embodiments, the selective ROCK2 inhibitor comprises Compound 453. In some embodiments, the selective ROCK2 inhibitor comprises Compound 454. In some embodiments, the selective ROCK2 inhibitor comprises Compound 455. In some embodiments, the selective ROCK2 inhibitor comprises Compound 456. In some embodiments, the selective ROCK2 inhibitor comprises Compound 457. In some embodiments, the selective ROCK2 inhibitor comprises Compound 458. In some embodiments, the selective ROCK2 inhibitor comprises Compound 459. In some embodiments, the selective ROCK2 inhibitor comprises Compound 460. In some embodiments, the selective ROCK2 inhibitor comprises Compound 461. In some embodiments, the selective ROCK2 inhibitor comprises Compound 462. In some embodiments, the selective ROCK2 inhibitor comprises Compound 463. In some embodiments, the selective ROCK2 inhibitor comprises Compound 464. In some embodiments, the selective ROCK2 inhibitor comprises Compound 465. In some embodiments, the selective ROCK2 inhibitor comprises Compound 466. In some embodiments, the selective ROCK2 inhibitor comprises Compound 467. In some embodiments, the selective ROCK2 inhibitor comprises Compound 468. In some embodiments, the selective ROCK2 inhibitor comprises Compound 469. In some embodiments, the selective ROCK2 inhibitor comprises Compound 470. In some embodiments, the selective ROCK2 inhibitor comprises Compound 471. In some embodiments, the selective ROCK2 inhibitor comprises Compound 472. In some embodiments, the selective ROCK2 inhibitor comprises Compound 473. In some embodiments, the selective ROCK2 inhibitor comprises Compound 474. In some embodiments, the selective ROCK2 inhibitor comprises Compound 475. In some embodiments, the selective ROCK2 inhibitor comprises Compound 476. In some embodiments, the selective ROCK2 inhibitor comprises Compound 477. In some embodiments, the selective ROCK2 inhibitor comprises Compound 478. In some embodiments, the selective ROCK2 inhibitor comprises Compound 479. In some embodiments, the selective ROCK2 inhibitor comprises Compound 480. In some embodiments, the selective ROCK2 inhibitor comprises Compound 481. In some embodiments, the selective ROCK2 inhibitor comprises Compound 482. In some embodiments, the selective ROCK2 inhibitor comprises Compound 483.

In some embodiments, the selective ROCK2 inhibitor is Compound 401. In some embodiments, the selective ROCK2 inhibitor is Compound 402. In some embodiments, the selective ROCK2 inhibitor is Compound 403. In some embodiments, the selective ROCK2 inhibitor is Compound 404. In some embodiments, the selective ROCK2 inhibitor is Compound 405. In some embodiments, the selective ROCK2 inhibitor is Compound 406. In some embodiments, the selective ROCK2 inhibitor is Compound 407. In some embodiments, the selective ROCK2 inhibitor is Compound 408. In some embodiments, the selective ROCK2 inhibitor is Compound 409. In some embodiments, the selective ROCK2 inhibitor is Compound 410. In some embodiments, the selective ROCK2 inhibitor is Compound 411. In some embodiments, the selective ROCK2 inhibitor is Compound 412. In some embodiments, the selective ROCK2 inhibitor is Compound 413. In some embodiments, the selective ROCK2 inhibitor is Compound 414. In some embodiments, the selective ROCK2 inhibitor is Compound 415. In some embodiments, the selective ROCK2 inhibitor is Compound 416. In some embodiments, the selective ROCK2 inhibitor is Compound 417. In some embodiments, the selective ROCK2 inhibitor is Compound 418. In some embodiments, the selective ROCK2 inhibitor is Compound 419. In some embodiments, the selective ROCK2 inhibitor is Compound 420. In some embodiments, the selective ROCK2 inhibitor is Compound 421. In some embodiments, the selective ROCK2 inhibitor is Compound 422. In some embodiments, the selective ROCK2 inhibitor is Compound 423. In some embodiments, the selective ROCK2 inhibitor is Compound 424. In some embodiments, the selective ROCK2 inhibitor is Compound 425. In some embodiments, the selective ROCK2 inhibitor is Compound 426. In some embodiments, the selective ROCK2 inhibitor is Compound 427. In some embodiments, the selective ROCK2 inhibitor is Compound 428. In some embodiments, the selective ROCK2 inhibitor is Compound 429. In some embodiments, the selective ROCK2 inhibitor is Compound 430. In some embodiments, the selective ROCK2 inhibitor is Compound 431. In some embodiments, the selective ROCK2 inhibitor is Compound 432. In some embodiments, the selective ROCK2 inhibitor is Compound 433. In some embodiments, the selective ROCK2 inhibitor is Compound 434. In some embodiments, the selective ROCK2 inhibitor is Compound 435. In some embodiments, the selective ROCK2 inhibitor is Compound 436. In some embodiments, the selective ROCK2 inhibitor is Compound 437. In some embodiments, the selective ROCK2 inhibitor is Compound 438. In some embodiments, the selective ROCK2 inhibitor is Compound 439. In some embodiments, the selective ROCK2 inhibitor is Compound 440. In some embodiments, the selective ROCK2 inhibitor is Compound 441. In some embodiments, the selective ROCK2 inhibitor is Compound 442. In some embodiments, the selective ROCK2 inhibitor is Compound 443. In some embodiments, the selective ROCK2 inhibitor is Compound 444. In some embodiments, the selective ROCK2 inhibitor is Compound 445. In some embodiments, the selective ROCK2 inhibitor is Compound 446. In some embodiments, the selective ROCK2 inhibitor is Compound 447. In some embodiments, the selective ROCK2 inhibitor is Compound 448. In some embodiments, the selective ROCK2 inhibitor is Compound 449. In some embodiments, the selective ROCK2 inhibitor is Compound 450. In some embodiments, the selective ROCK2 inhibitor is Compound 451. In some embodiments, the selective ROCK2 inhibitor is Compound 452. In some embodiments, the selective ROCK2 inhibitor is Compound 453. In some embodiments, the selective ROCK2 inhibitor is Compound 454. In some embodiments, the selective ROCK2 inhibitor is Compound 455. In some embodiments, the selective ROCK2 inhibitor is Compound 456. In some embodiments, the selective ROCK2 inhibitor is Compound 457. In some embodiments, the selective ROCK2 inhibitor is Compound 458. In some embodiments, the selective ROCK2 inhibitor is Compound 459. In some embodiments, the selective ROCK2 inhibitor is Compound 460. In some embodiments, the selective ROCK2 inhibitor is Compound 461. In some embodiments, the selective ROCK2 inhibitor is Compound 462. In some embodiments, the selective ROCK2 inhibitor is Compound 463. In some embodiments, the selective ROCK2 inhibitor is Compound 464. In some embodiments, the selective ROCK2 inhibitor is Compound 465. In some embodiments, the selective ROCK2 inhibitor is Compound 466. In some embodiments, the selective ROCK2 inhibitor is Compound 467. In some embodiments, the selective ROCK2 inhibitor is Compound 468. In some embodiments, the selective ROCK2 inhibitor is Compound 469. In some embodiments, the selective ROCK2 inhibitor is Compound 470. In some embodiments, the selective ROCK2 inhibitor is Compound 471. In some embodiments, the selective ROCK2 inhibitor is Compound 472. In some embodiments, the selective ROCK2 inhibitor is Compound 473. In some embodiments, the selective ROCK2 inhibitor is Compound 474. In some embodiments, the selective ROCK2 inhibitor is Compound 475. In some embodiments, the selective ROCK2 inhibitor is Compound 476. In some embodiments, the selective ROCK2 inhibitor is Compound 477. In some embodiments, the selective ROCK2 inhibitor is Compound 478. In some embodiments, the selective ROCK2 inhibitor is Compound 479. In some embodiments, the selective ROCK2 inhibitor is Compound 480. In some embodiments, the selective ROCK2 inhibitor is Compound 481. In some embodiments, the selective ROCK2 inhibitor is Compound 482. In some embodiments, the selective ROCK2 inhibitor is Compound 483.

In some embodiments, the selective ROCK2 small molecule inhibitor is selected from Compounds 484 and 485 of Table 4. In some embodiments, the selective ROCK2 small molecule inhibitor is selected from Compounds 484 and 485 of Table 4, and pharmaceutically acceptable salts, stereoisomers, tautomers, co-crystals, polymorphs, solvates, hydrates, prodrugs, and isotopically labeled derivatives thereof. In some embodiments, the selective ROCK2 small molecule inhibitor is selected from Compounds 484 and 485 of Table 4, and pharmaceutically acceptable salts thereof.

TABLE 4
Examples of Selective ROCK2 Inhibitors

| Compound | Structure |
|---|---|
| 484 | 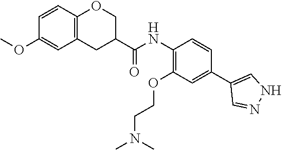 [SMILES: COc1ccc2c(c1)CC(C(=O)Nc1ccc(-c3cn[nH]c3)cc1OCCN(C)C)CO2] |
| 485 | 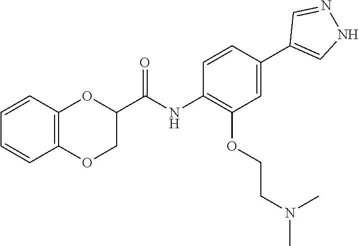 [SMILES: CN(C)CCOc1cc(-c2cn[nH]c2)ccc1NC(=O)C1COc2ccccc2O1] |

In some embodiments, the selective ROCK2 inhibitor comprises Compound 484. In some embodiments, the selective ROCK2 inhibitor comprises Compound 485.

In some embodiments, the selective ROCK2 inhibitor is Compound 484. In some embodiments, the selective ROCK2 inhibitor is Compound 485.

Other non-limiting examples of selective ROCK2 inhibitors are provided in Table 5 and in U.S. Patent Publication No. US20190276440A1, incorporated by reference herein in its entirety. In some embodiments, the selective ROCK2 small molecule inhibitor is a compound of Formula (I) (i.e., including any subgenera or species thereof) disclosed in U.S. Patent Publication No. US20190276440A1, incorporated by reference herein in its entirety; or a pharmaceutically acceptable salt, stereoisomer, tautomer, isotopically labeled derivative, solvate, hydrate, polymorph, co-crystal, or prodrug thereof. In some embodiments, the selective ROCK2 small molecule inhibitor is a compound of Formula (I) (i.e., including any subgenera or species thereof) disclosed in U.S. Patent Publication No. US20190276440A1, incorporated by reference herein in its entirety; or a pharmaceutically acceptable salt thereof.

In some embodiments, the selective ROCK2 small molecule inhibitor comprises a compound selected from Compounds 486-559 of Table 5. In some embodiments, the selective ROCK2 small molecule inhibitor is selected from Compounds 486-559 of Table 5. In some embodiments, the selective ROCK2 small molecule inhibitor is any one of Compounds 486-559 of Table 5, or a pharmaceutically acceptable salt, stereoisomer, tautomer, isotopically labeled derivative, solvate, hydrate, polymorph, co-crystal, or prodrug thereof. In some embodiments, the selective ROCK2 small molecule inhibitor is any one of Compounds 486-559 of Table 5, or a pharmaceutically acceptable salt thereof. In instances where a particular salt form (e.g., trifluoroacetic acid (TFA) salt) of a compound is shown in Table 5, other pharmaceutically acceptable salts of the compound are also provided as part of the present disclosure.

TABLE 5
Examples of Selective ROCK2 Inhibitors

| # | Structure |
|---|---|
| 486 | 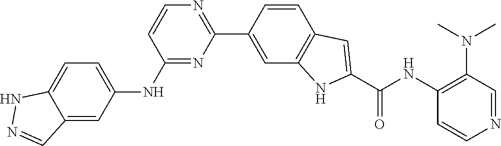 [SMILES: CN(C)c1cnccc1NC(=O)c1cc2ccc(-c3nccc(Nc4ccc5[nH]ncc5c4)n3)cc2[nH]1] |
| 487 | 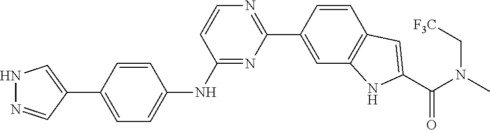 [SMILES: CN(CC(F)(F)F)C(=O)c1cc2ccc(-c3nccc(Nc4ccc(-c5cn[nH]c5)cc4)n3)cc2[nH]1] |
| 488 | 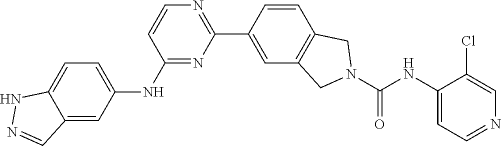 [SMILES: O=C(Nc1ccncc1Cl)N1Cc2ccc(-c3nccc(Nc4ccc5[nH]ncc5c4)n3)cc2C1] |
| 489 | 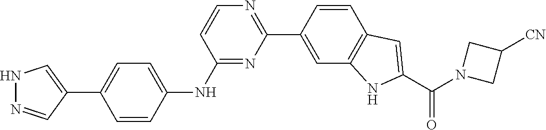 [SMILES: N#CC1CN(C(=O)c2cc3ccc(-c4nccc(Nc5ccc(-c6cn[nH]c6)cc5)n4)cc3[nH]2)C1] |
| 490 | 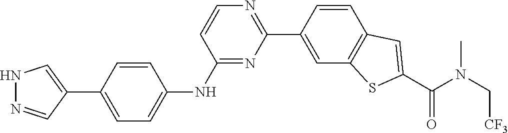 [SMILES: CN(CC(F)(F)F)C(=O)c1cc2ccc(-c3nccc(Nc4ccc(-c5cn[nH]c5)cc4)n3)cc2s1] |
| 491 | 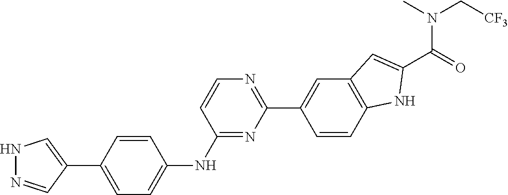 [SMILES: CN(CC(F)(F)F)C(=O)c1cc2cc(-c3nccc(Nc4ccc(-c5cn[nH]c5)cc4)n3)ccc2[nH]1] |
| 492 | 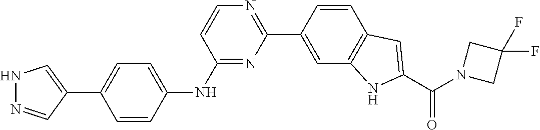 [SMILES: O=C(c1cc2ccc(-c3nccc(Nc4ccc(-c5cn[nH]c5)cc4)n3)cc2[nH]1)N1CC(F)(F)C1] |
| 493 | 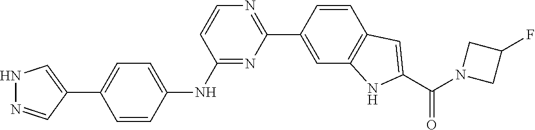 [SMILES: O=C(c1cc2ccc(-c3nccc(Nc4ccc(-c5cn[nH]c5)cc4)n3)cc2[nH]1)N1CC(F)C1] |
| 494 | 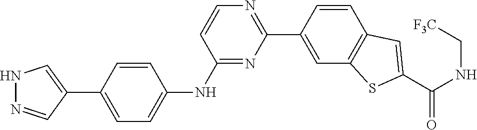 [SMILES: O=C(NCC(F)(F)F)c1cc2ccc(-c3nccc(Nc4ccc(-c5cn[nH]c5)cc4)n3)cc2s1] |
| 495 | 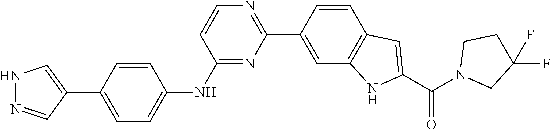 [SMILES: O=C(c1cc2ccc(-c3nccc(Nc4ccc(-c5cn[nH]c5)cc4)n3)cc2[nH]1)N1CCC(F)(F)C1] |
| 496 | 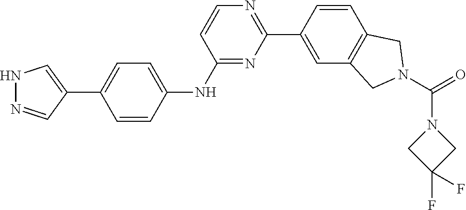 [SMILES: O=C(N1Cc2ccc(-c3nccc(Nc4ccc(-c5cn[nH]c5)cc4)n3)cc2C1)N1CC(F)(F)C1] |
| 497 | 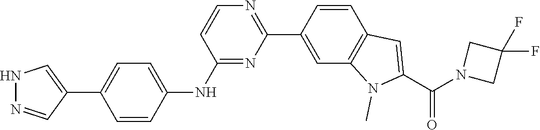 [SMILES: Cn1c(C(=O)N2CC(F)(F)C2)cc2ccc(-c3nccc(Nc4ccc(-c5cn[nH]c5)cc4)n3)cc21] |
| 498 | 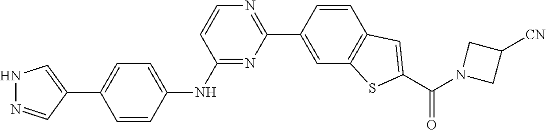 [SMILES: N#CC1CN(C(=O)c2cc3ccc(-c4nccc(Nc5ccc(-c6cn[nH]c6)cc5)n4)cc3s2)C1] |
| 499 | 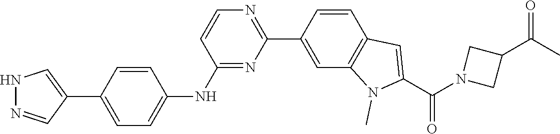 [SMILES: CC(=O)C1CN(C(=O)c2cc3ccc(-c4nccc(Nc5ccc(-c6cn[nH]c6)cc5)n4)cc3n2C)C1] |
| 500 | 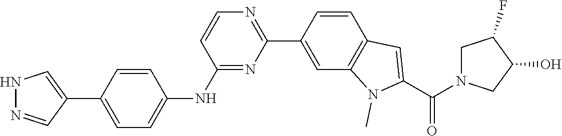 [SMILES: Cn1c(C(=O)N2C[C@@H](O)[C@@H](F)C2)cc2ccc(-c3nccc(Nc4ccc(-c5cn[nH]c5)cc4)n3)cc21] |
| 501 | 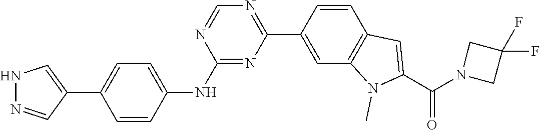 [SMILES: Cn1c(C(=O)N2CC(F)(F)C2)cc2ccc(-c3ncnc(Nc4ccc(-c5cn[nH]c5)cc4)n3)cc21] |
| 502 | 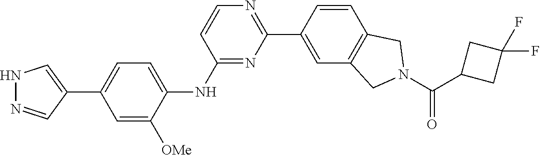 [SMILES: COc1cc(-c2cn[nH]c2)ccc1Nc1ccnc(-c2ccc3c(c2)CN(C(=O)C2CC(F)(F)C2)C3)n1] |
| 503 | 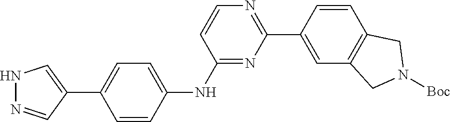 [SMILES: CC(C)(C)OC(=O)N1Cc2ccc(-c3nccc(Nc4ccc(-c5cn[nH]c5)cc4)n3)cc2C1] |
| 504 | 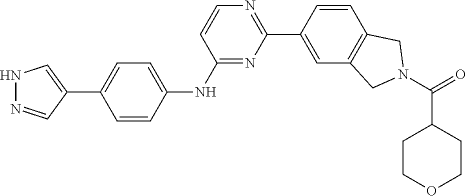 [SMILES: O=C(C1CCOCC1)N1Cc2ccc(-c3nccc(Nc4ccc(-c5cn[nH]c5)cc4)n3)cc2C1] |
| 505 | 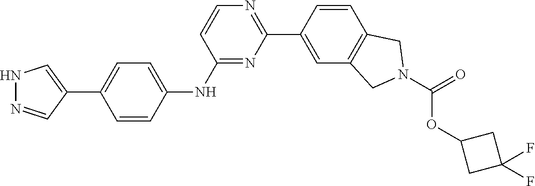 [SMILES: O=C(OC1CC(F)(F)C1)N1Cc2ccc(-c3nccc(Nc4ccc(-c5cn[nH]c5)cc4)n3)cc2C1] |
| 506 | 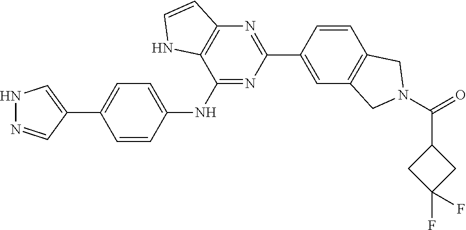 [SMILES: O=C(C1CC(F)(F)C1)N1Cc2ccc(-c3nc(Nc4ccc(-c5cn[nH]c5)cc4)c4[nH]ccc4n3)cc2C1] |
| 507 | 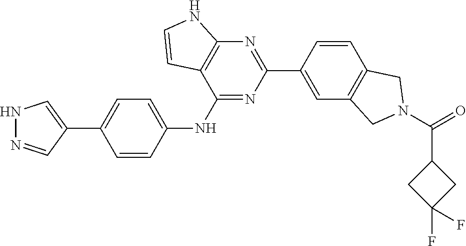 [SMILES: O=C(C1CC(F)(F)C1)N1Cc2ccc(-c3nc(Nc4ccc(-c5cn[nH]c5)cc4)c4cc[nH]c4n3)cc2C1] |
| 508 | 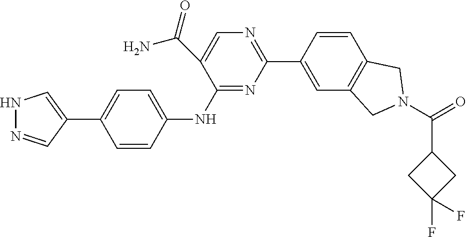 [SMILES: NC(=O)c1cnc(-c2ccc3c(c2)CN(C(=O)C2CC(F)(F)C2)C3)nc1Nc1ccc(-c2cn[nH]c2)cc1] |
| 509 | 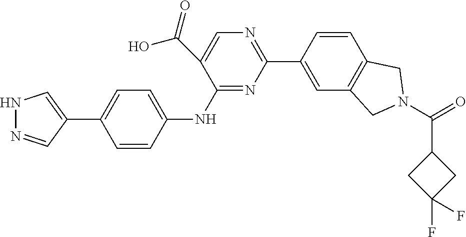 [SMILES: O=C(O)c1cnc(-c2ccc3c(c2)CN(C(=O)C2CC(F)(F)C2)C3)nc1Nc1ccc(-c2cn[nH]c2)cc1] |
| 510 | 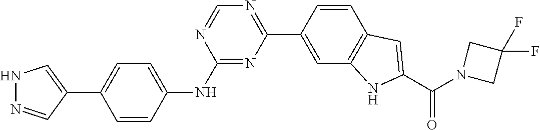 [SMILES: O=C(c1cc2ccc(-c3ncnc(Nc4ccc(-c5cn[nH]c5)cc4)n3)cc2[nH]1)N1CC(F)(F)C1] |
| 511 | 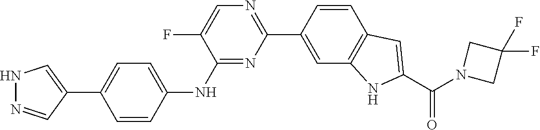 [SMILES: O=C(c1cc2ccc(-c3ncc(F)c(Nc4ccc(-c5cn[nH]c5)cc4)n3)cc2[nH]1)N1CC(F)(F)C1] |
| 512 | 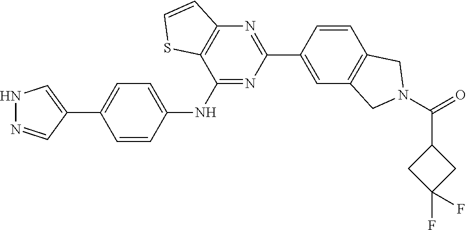 [SMILES: O=C(C1CC(F)(F)C1)N1Cc2ccc(-c3nc(Nc4ccc(-c5cn[nH]c5)cc4)c4sccc4n3)cc2C1] |
| 513 | 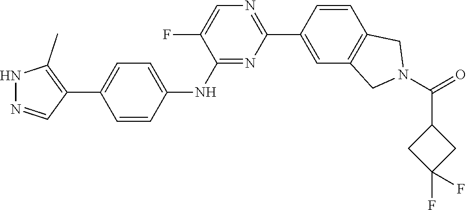 [SMILES: Cc1[nH]ncc1-c1ccc(Nc2nc(-c3ccc4c(c3)CN(C(=O)C3CC(F)(F)C3)C4)ncc2F)cc1] |
| 514 | 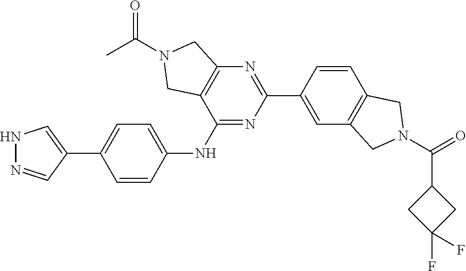 [SMILES: CC(=O)N1Cc2nc(-c3ccc4c(c3)CN(C(=O)C3CC(F)(F)C3)C4)nc(Nc3ccc(-c4cn[nH]c4)cc3)c2C1] |
| 515 | 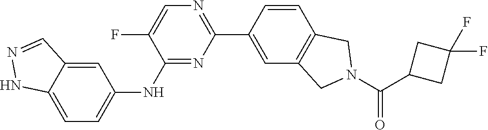 [SMILES: O=C(C1CC(F)(F)C1)N1Cc2ccc(-c3ncc(F)c(Nc4ccc5[nH]ncc5c4)n3)cc2C1] |
| 516 | 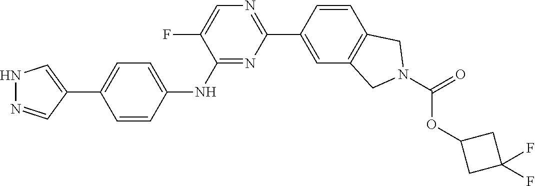 [SMILES: O=C(OC1CC(F)(F)C1)N1Cc2ccc(-c3ncc(F)c(Nc4ccc(-c5cn[nH]c5)cc4)n3)cc2C1] |
| 517 | 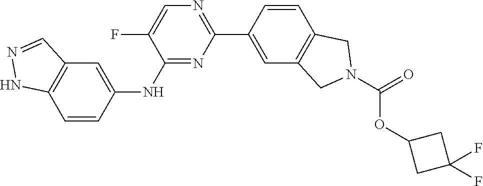 [SMILES: O=C(OC1CC(F)(F)C1)N1Cc2ccc(-c3ncc(F)c(Nc4ccc5[nH]ncc5c4)n3)cc2C1] |
| 518 | 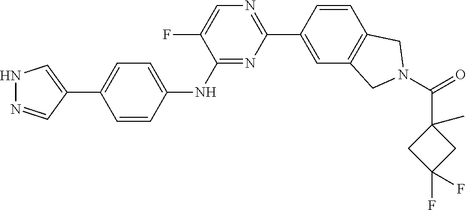 [SMILES: CC1(C(=O)N2Cc3ccc(-c4ncc(F)c(Nc5ccc(-c6cn[nH]c6)cc5)n4)cc3C2)CC(F)(F)C1] |
| 519 | 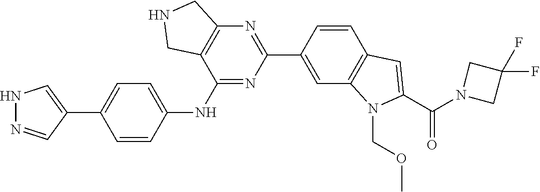 [SMILES: COCn1c(C(=O)N2CC(F)(F)C2)cc2ccc(-c3nc4c(c(Nc5ccc(-c6cn[nH]c6)cc5)n3)CNC4)cc21] |
| 520 | 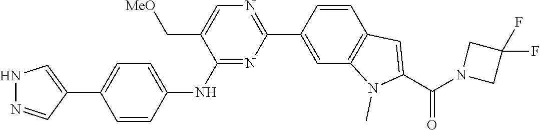 [SMILES: COCc1cnc(-c2ccc3cc(C(=O)N4CC(F)(F)C4)n(C)c3c2)nc1Nc1ccc(-c2cn[nH]c2)cc1] |
| 521 | 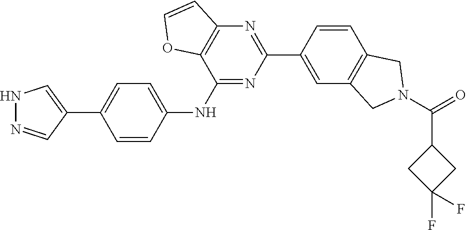 [SMILES: O=C(C1CC(F)(F)C1)N1Cc2ccc(-c3nc(Nc4ccc(-c5cn[nH]c5)cc4)c4occc4n3)cc2C1] |
| 522 | 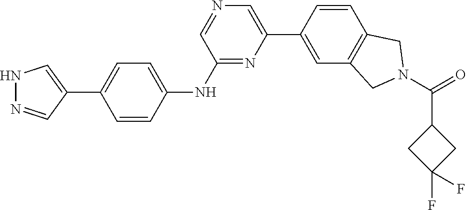 [SMILES: O=C(C1CC(F)(F)C1)N1Cc2ccc(-c3cncc(Nc4ccc(-c5cn[nH]c5)cc4)n3)cc2C1] |
| 523 | 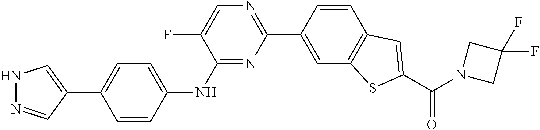 [SMILES: O=C(c1cc2ccc(-c3ncc(F)c(Nc4ccc(-c5cn[nH]c5)cc4)n3)cc2s1)N1CC(F)(F)C1] |
| 524 | 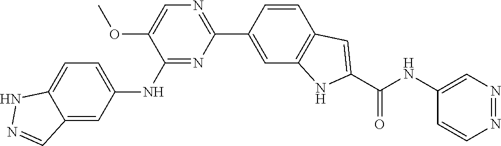 [SMILES: COc1cnc(-c2ccc3cc(C(=O)Nc4ccnnc4)[nH]c3c2)nc1Nc1ccc2[nH]ncc2c1] |
| 525 | 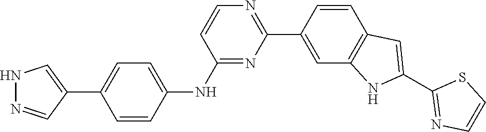 [SMILES: c1cc(Nc2ccc(-c3cn[nH]c3)cc2)nc(-c2ccc3cc(-c4nccs4)[nH]c3c2)n1] |
| 526 | 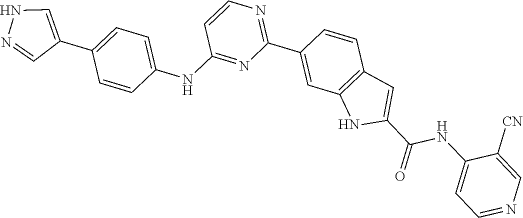 [SMILES: N#Cc1cnccc1NC(=O)c1cc2ccc(-c3nccc(Nc4ccc(-c5cn[nH]c5)cc4)n3)cc2[nH]1] |
| 527 | 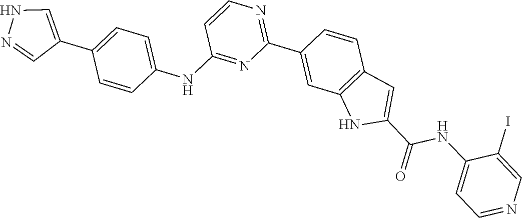 [SMILES: O=C(Nc1ccncc1I)c1cc2ccc(-c3nccc(Nc4ccc(-c5cn[nH]c5)cc4)n3)cc2[nH]1] |
| 528 | 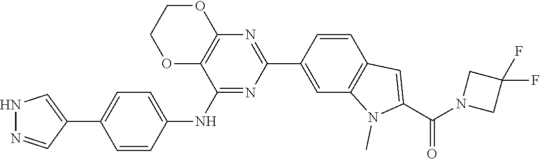 [SMILES: Cn1c(C(=O)N2CC(F)(F)C2)cc2ccc(-c3nc(Nc4ccc(-c5cn[nH]c5)cc4)c4c(n3)OCCO4)cc21] |
| 529 | 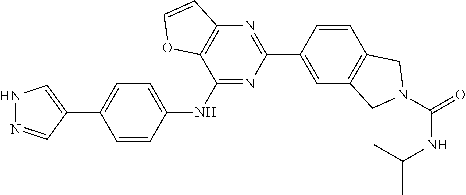 [SMILES: CC(C)NC(=O)N1Cc2ccc(-c3nc(Nc4ccc(-c5cn[nH]c5)cc4)c4occc4n3)cc2C1] |
| 530 | 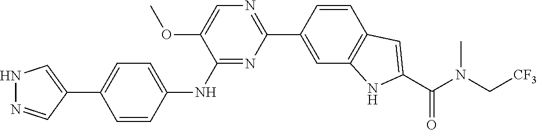 [SMILES: COc1cnc(-c2ccc3cc(C(=O)N(C)CC(F)(F)F)[nH]c3c2)nc1Nc1ccc(-c2cn[nH]c2)cc1] |
| 531 | 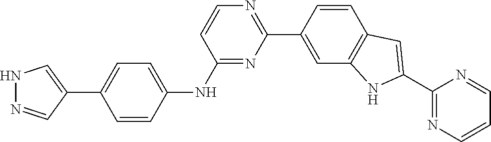 [SMILES: c1cnc(-c2cc3ccc(-c4nccc(Nc5ccc(-c6cn[nH]c6)cc5)n4)cc3[nH]2)nc1] |
| 532 | 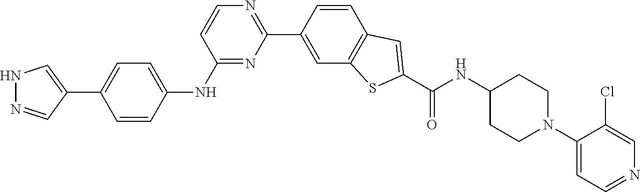 [SMILES: O=C(NC1CCN(c2ccncc2Cl)CC1)c1cc2ccc(-c3nccc(Nc4ccc(-c5cn[nH]c5)cc4)n3)cc2s1] |
| 533 | 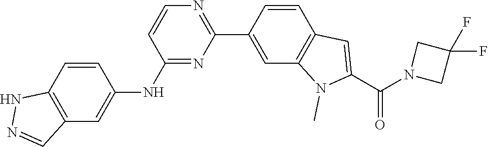 [SMILES: Cn1c(C(=O)N2CC(F)(F)C2)cc2ccc(-c3nccc(Nc4ccc5[nH]ncc5c4)n3)cc21] |
| 534 | 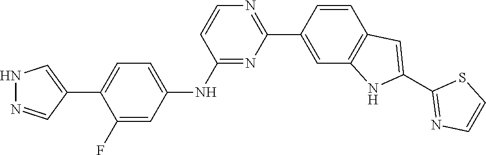 [SMILES: Fc1cc(Nc2ccnc(-c3ccc4cc(-c5nccs5)[nH]c4c3)n2)ccc1-c1cn[nH]c1] |
| 535 | 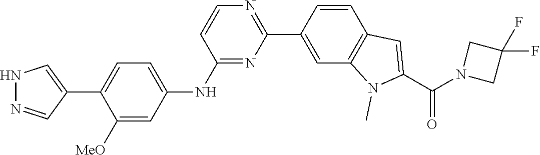 [SMILES: COc1cc(Nc2ccnc(-c3ccc4cc(C(=O)N5CC(F)(F)C5)n(C)c4c3)n2)ccc1-c1cn[nH]c1] |
| 536 | 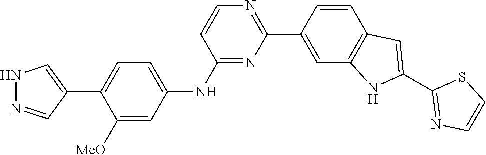 [SMILES: COc1cc(Nc2ccnc(-c3ccc4cc(-c5nccs5)[nH]c4c3)n2)ccc1-c1cn[nH]c1] |
| 537 | 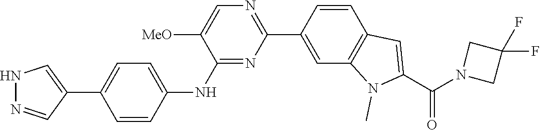 [SMILES: COc1cnc(-c2ccc3cc(C(=O)N4CC(F)(F)C4)n(C)c3c2)nc1Nc1ccc(-c2cn[nH]c2)cc1] |
| 538 | 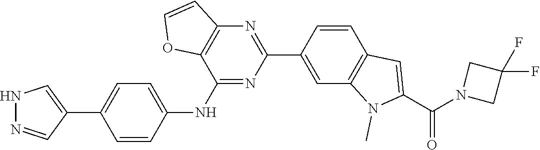 [SMILES: Cn1c(C(=O)N2CC(F)(F)C2)cc2ccc(-c3nc(Nc4ccc(-c5cn[nH]c5)cc4)c4occc4n3)cc21] |
| 539 | 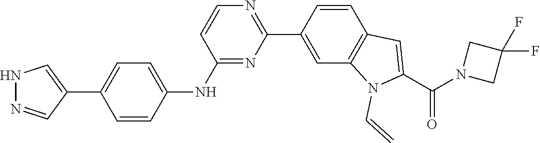 [SMILES: C=Cn1c(C(=O)N2CC(F)(F)C2)cc2ccc(-c3nccc(Nc4ccc(-c5cn[nH]c5)cc4)n3)cc21] |
| 540 | 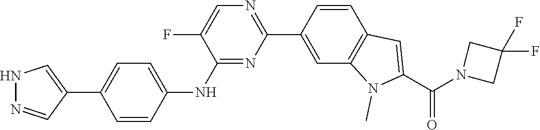 [SMILES: Cn1c(C(=O)N2CC(F)(F)C2)cc2ccc(-c3ncc(F)c(Nc4ccc(-c5cn[nH]c5)cc4)n3)cc21] |
| 541 | 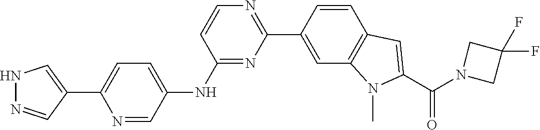 [SMILES: Cn1c(C(=O)N2CC(F)(F)C2)cc2ccc(-c3nccc(Nc4ccc(-c5cn[nH]c5)nc4)n3)cc21] |
| 542 | 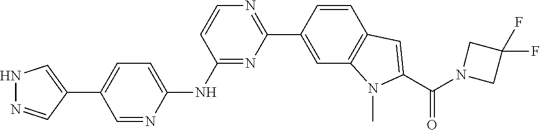 [SMILES: Cn1c(C(=O)N2CC(F)(F)C2)cc2ccc(-c3nccc(Nc4ccc(-c5cn[nH]c5)cn4)n3)cc21] |
| 543 | 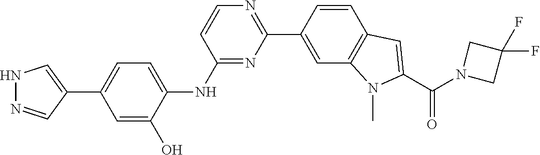 [SMILES: Cn1c(C(=O)N2CC(F)(F)C2)cc2ccc(-c3nccc(Nc4ccc(-c5cn[nH]c5)cc4O)n3)cc21] |
| 544 | 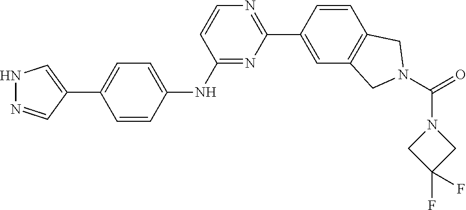 [SMILES: O=C(N1Cc2ccc(-c3nccc(Nc4ccc(-c5cn[nH]c5)cc4)n3)cc2C1)N1CC(F)(F)C1] |
| 545 | 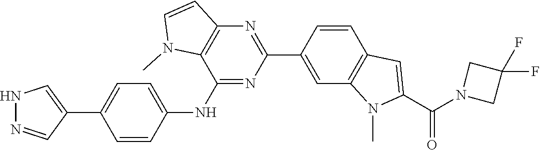 [SMILES: Cn1ccc2nc(-c3ccc4cc(C(=O)N5CC(F)(F)C5)n(C)c4c3)nc(Nc3ccc(-c4cn[nH]c4)cc3)c21] |
| 546 | 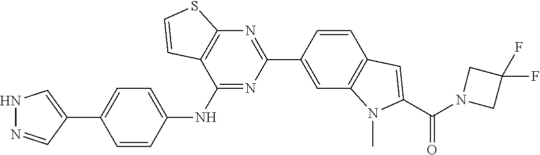 [SMILES: Cn1c(C(=O)N2CC(F)(F)C2)cc2ccc(-c3nc(Nc4ccc(-c5cn[nH]c5)cc4)c4ccsc4n3)cc21] |
| 547 | 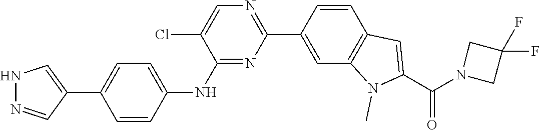 [SMILES: Cn1c(C(=O)N2CC(F)(F)C2)cc2ccc(-c3ncc(Cl)c(Nc4ccc(-c5cn[nH]c5)cc4)n3)cc21] |
| 548 | 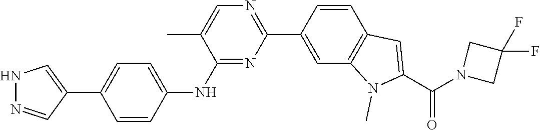 [SMILES: Cc1cnc(-c2ccc3cc(C(=O)N4CC(F)(F)C4)n(C)c3c2)nc1Nc1ccc(-c2cn[nH]c2)cc1] |
| 549 | 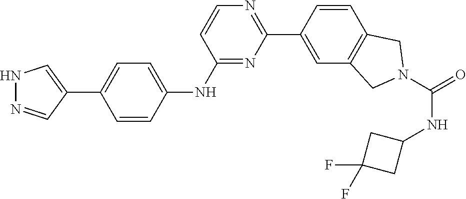 [SMILES: O=C(NC1CC(F)(F)C1)N1Cc2ccc(-c3nccc(Nc4ccc(-c5cn[nH]c5)cc4)n3)cc2C1] |
| 550 | 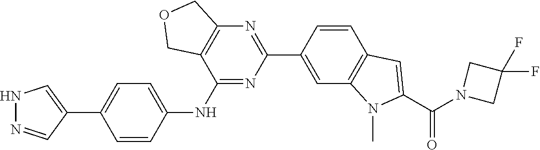 [SMILES: Cn1c(C(=O)N2CC(F)(F)C2)cc2ccc(-c3nc4c(c(Nc5ccc(-c6cn[nH]c6)cc5)n3)COC4)cc21] |
| 551 | 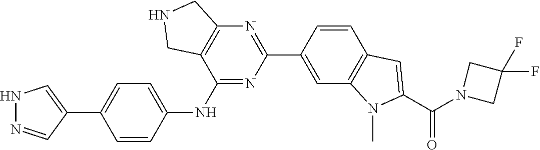 [SMILES: Cn1c(C(=O)N2CC(F)(F)C2)cc2ccc(-c3nc4c(c(Nc5ccc(-c6cn[nH]c6)cc5)n3)CNC4)cc21] |
| 552 | 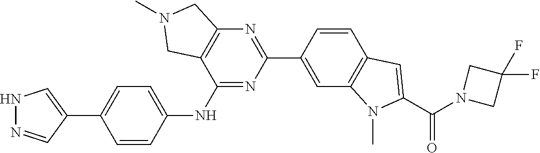 [SMILES: CN1Cc2nc(-c3ccc4cc(C(=O)N5CC(F)(F)C5)n(C)c4c3)nc(Nc3ccc(-c4cn[nH]c4)cc3)c2C1] |
| 553 | 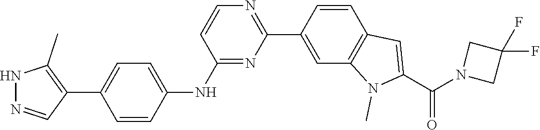 [SMILES: Cc1[nH]ncc1-c1ccc(Nc2ccnc(-c3ccc4cc(C(=O)N5CC(F)(F)C5)n(C)c4c3)n2)cc1] |
| 554 | 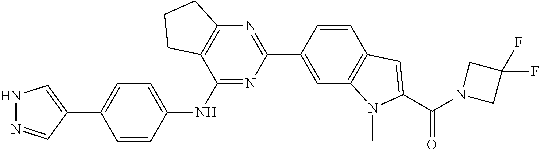 [SMILES: Cn1c(C(=O)N2CC(F)(F)C2)cc2ccc(-c3nc4c(c(Nc5ccc(-c6cn[nH]c6)cc5)n3)CCC4)cc21] |
| 555 | 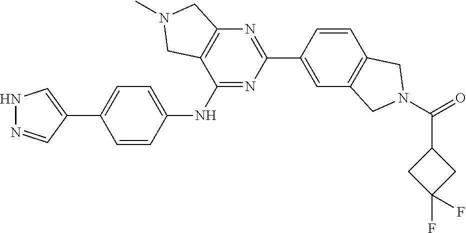 [SMILES: CN1Cc2nc(-c3ccc4c(c3)CN(C(=O)C3CC(F)(F)C3)C4)nc(Nc3ccc(-c4cn[nH]c4)cc3)c2C1] |
| 556 | 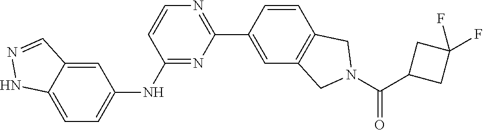 [SMILES: O=C(C1CC(F)(F)C1)N1Cc2ccc(-c3nccc(Nc4ccc5[nH]ncc5c4)n3)cc2C1] |
| 557 | 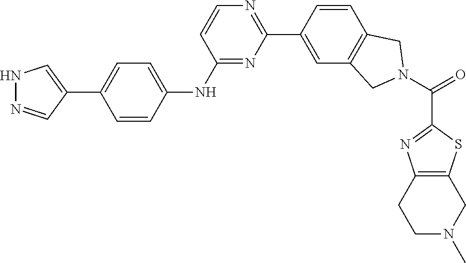 [SMILES: CN1CCc2nc(C(=O)N3Cc4ccc(-c5nccc(Nc6ccc(-c7cn[nH]c7)cc6)n5)cc4C3)sc2C1] |
| 558 | 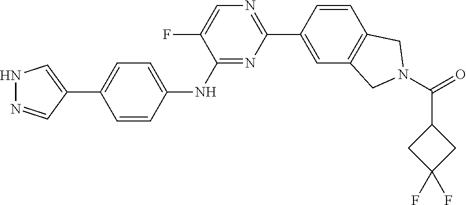 [SMILES: O=C(C1CC(F)(F)C1)N1Cc2ccc(-c3ncc(F)c(Nc4ccc(-c5cn[nH]c5)cc4)n3)cc2C1] |
| 559 | 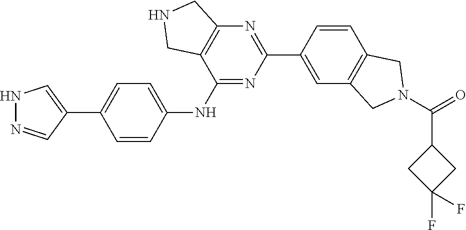 [SMILES: O=C(C1CC(F)(F)C1)N1Cc2ccc(-c3nc4c(c(Nc5ccc(-c6cn[nH]c6)cc5)n3)CNC4)cc2C1] |

# = Compound

In some embodiments, the selective ROCK2 inhibitor comprises Compound 486. In some embodiments, the selective ROCK2 inhibitor comprises Compound 487. In some embodiments, the selective ROCK2 inhibitor comprises Compound 488. In some embodiments, the selective ROCK2 inhibitor comprises Compound 489. In some embodiments, the selective ROCK2 inhibitor comprises Compound 490. In some embodiments, the selective ROCK2 inhibitor comprises Compound 491. In some embodiments, the selective ROCK2 inhibitor comprises Compound 492. In some embodiments, the selective ROCK2 inhibitor comprises Compound 493. In some embodiments, the selective ROCK2 inhibitor comprises Compound 494. In some embodiments, the selective ROCK2 inhibitor comprises Compound 495. In some embodiments, the selective ROCK2 inhibitor comprises Compound 496. In some embodiments, the selective ROCK2 inhibitor comprises Compound 497. In some embodiments, the selective ROCK2 inhibitor comprises Compound 498. In some embodiments, the selective ROCK2 inhibitor comprises Compound 499. In some embodiments, the selective ROCK2 inhibitor comprises Compound 500. In some embodiments, the selective ROCK2 inhibitor comprises Compound 501. In some embodiments, the selective ROCK2 inhibitor comprises Compound 502. In some embodiments, the selective ROCK2 inhibitor comprises Compound 503. In some embodiments, the selective ROCK2 inhibitor comprises Compound 504. In some embodiments, the selective ROCK2 inhibitor comprises Compound 505. In some embodiments, the selective ROCK2 inhibitor comprises Compound 506. In some embodiments, the selective ROCK2 inhibitor comprises Compound 507. In some embodiments, the selective ROCK2 inhibitor comprises Compound 508. In some embodiments, the selective ROCK2 inhibitor comprises Compound 509. In some embodiments, the selective ROCK2 inhibitor comprises Compound 510. In some embodiments, the selective ROCK2 inhibitor comprises Compound 511. In some embodiments, the selective ROCK2 inhibitor comprises Compound 512. In some embodiments, the selective ROCK2 inhibitor comprises Compound 513. In some embodiments, the selective ROCK2 inhibitor comprises Compound 514. In some embodiments, the selective ROCK2 inhibitor comprises Compound 515. In some embodiments, the selective ROCK2 inhibitor comprises Compound 516. In some embodiments, the selective ROCK2 inhibitor comprises Compound 517. In some embodiments, the selective ROCK2 inhibitor comprises Compound 518. In some embodiments, the selective ROCK2 inhibitor comprises Compound 519. In some embodiments, the selective ROCK2 inhibitor comprises Compound 520. In some embodiments, the selective ROCK2 inhibitor comprises Compound 521. In some embodiments, the selective ROCK2 inhibitor comprises Compound 522. In some embodiments, the selective ROCK2 inhibitor comprises Compound 523. In some embodiments, the selective ROCK2 inhibitor comprises Compound 524. In some embodiments, the selective ROCK2 inhibitor comprises Compound 525. In some embodiments, the selective ROCK2 inhibitor comprises Compound 526. In some embodiments, the selective ROCK2 inhibitor comprises Compound 527. In some embodiments, the selective ROCK2 inhibitor comprises Compound 528. In some embodiments, the selective ROCK2 inhibitor comprises Compound 529. In some embodiments, the selective ROCK2 inhibitor comprises Compound 530. In some embodiments, the selective ROCK2 inhibitor comprises Compound 531. In some embodiments, the selective ROCK2 inhibitor comprises Compound 532. In some embodiments, the selective ROCK2 inhibitor comprises Compound 533. In some embodiments, the selective ROCK2 inhibitor comprises Compound 534. In some embodiments, the selective ROCK2 inhibitor comprises Compound 535. In some embodiments, the selective ROCK2 inhibitor comprises Compound 536. In some embodiments, the selective ROCK2 inhibitor comprises Compound 537. In some embodiments, the selective ROCK2 inhibitor comprises Compound 538. In some embodiments, the selective ROCK2 inhibitor comprises Compound 539. In some embodiments, the selective ROCK2 inhibitor comprises Compound 540. In some embodiments, the selective ROCK2 inhibitor comprises Compound 541. In some embodiments, the selective ROCK2 inhibitor comprises Compound 542. In some embodiments, the selective ROCK2 inhibitor comprises Compound 543. In some embodiments, the selective ROCK2 inhibitor comprises Compound 544. In some embodiments, the selective ROCK2 inhibitor comprises Compound 545. In some embodiments, the selective ROCK2 inhibitor comprises Compound 546. In some embodiments, the selective ROCK2 inhibitor comprises Compound 547. In some embodiments, the selective ROCK2 inhibitor comprises Compound 548. In some embodiments, the selective ROCK2 inhibitor comprises Compound 549. In some embodiments, the selective ROCK2 inhibitor comprises Compound 550. In some embodiments, the selective ROCK2 inhibitor comprises Compound 551. In some embodiments, the selective ROCK2 inhibitor comprises Compound 552. In some embodiments, the selective ROCK2 inhibitor comprises Compound 553. In some embodiments, the selective ROCK2 inhibitor comprises Compound 554. In some embodiments, the selective ROCK2 inhibitor comprises Compound 555. In some embodiments, the selective ROCK2 inhibitor comprises Compound 556. In some embodiments, the selective ROCK2 inhibitor comprises Compound 557. In some embodiments, the selective ROCK2 inhibitor comprises Compound 558. In some embodiments, the selective ROCK2 inhibitor comprises Compound 559.

A selective ROCK2 inhibitor may be administered (delivered) to a subject by any route appropriate for the dosage form of the inhibitor. For example, a selective ROCK2 inhibitor may be formulated as an intravenous solution and delivered to a subject via intravenous administration. As another example, a ROCK2 inhibitor may be formulated as a tablet (or other pill form) or as a liquid suspension and delivered to a subject via oral administration. Other dosage forms and delivery routes, such as but not limited to, intranasal and intramuscular are contemplated herein.

Formulation, generally, refers to the process of combing an agent, such as a selective ROCK2 inhibitor, with carriers, excipients, and/or other inert agents to produce a therapeutic composition. See, e.g., Remington: The Science and Practice of Pharmacy, 19* Edition (1995) and/or in Handbook of Pharmaceutical Granulation Technology, Chapter 7, “Drugs and the Pharmaceutical Sciences”, vol. 81, 1997.

### Biomimetic Model

Also provided herein are biomimetic models of lymphatic vessels and methods of use. In some embodiments, the models comprise a three-dimensional collagen hydrogel comprising a polydimethylsiloxane housing and two parallel cylindrical channels (see, e.g., Nguyen D T et al. PNAS 2013; 110(17): 6712-6717). In some embodiments, human dermal lymphatic endothelial cells are seeded in one of the cylindrical channels. In some embodiments, the model further comprises lymphangiogenic vascular endothelial growth factor C or other growth factor. In some embodiments, the biomimetic model is used to assess lymphatics or lymphatic drainage, for example, drainage of interstitial fluid. Thus, provided herein are methods of using the biomimetic model of lymphatic vessels to measure drainage, such as lymphatic drainage.

In some embodiments, the biomimetic models comprise at least one (e.g., 1, 2, 3, 4, or more) channel, for example, on a gel (e.g., collagen gel, fibrin, materiel, synthetic hydrogel, or hydrogel made from other ECM components). In some embodiments, the at least one channel is housed in a material selected from polydimethylsiloxane (PDMS), polypropylene, polystyrene, polyurethanes, polycaprolactone, polymethylmethacrylate, and polyacetate.

### EXAMPLES

Animal models of lymphedema are difficult to use to identify the pathophysiologic mechanisms underlying this multifactorial disease, because it is difficult to isolate the relative contributions of biological and biophysical factors. In contrast, two-dimensional (2D) cell culture models are highly controllable but do not recapitulate the 3D organization of lymphatics in vivo.

### Example 1. Development of an In Vitro Lymphatic Drainage Model

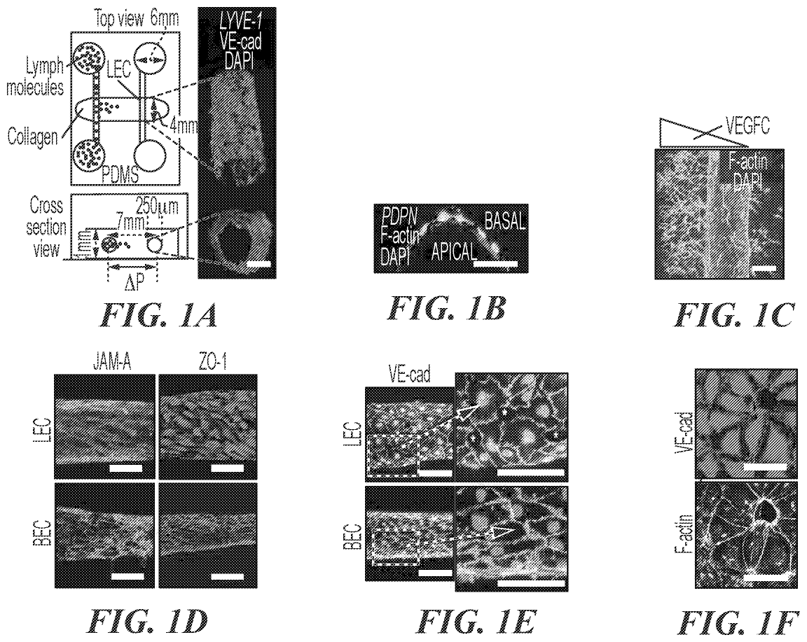

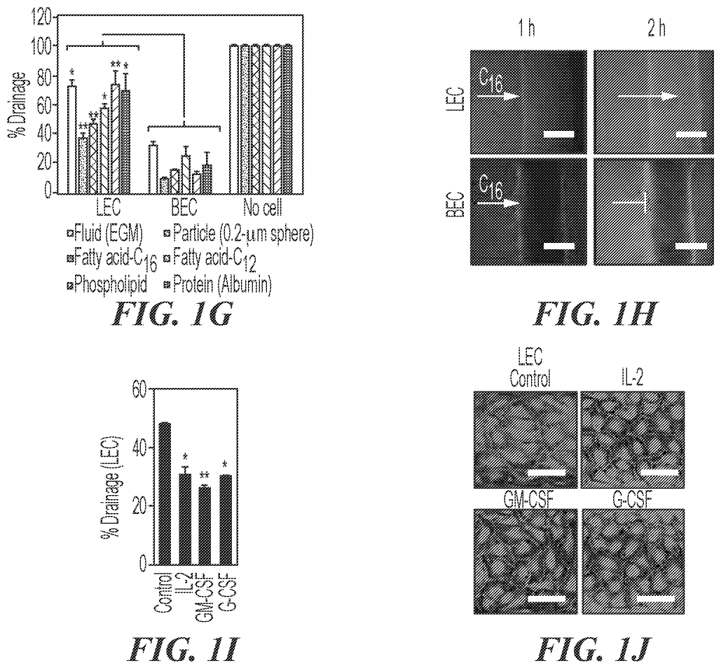

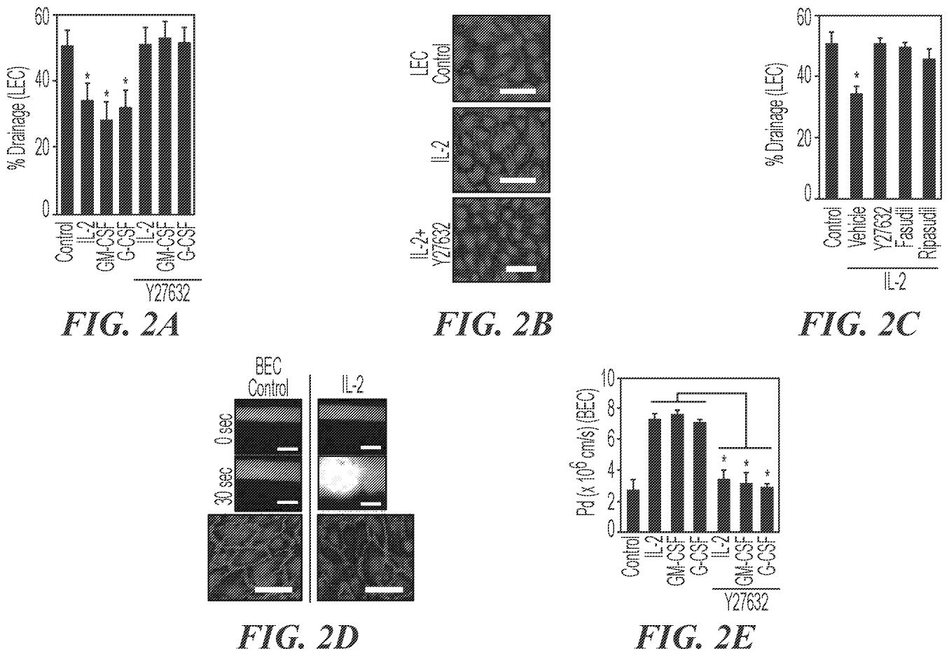

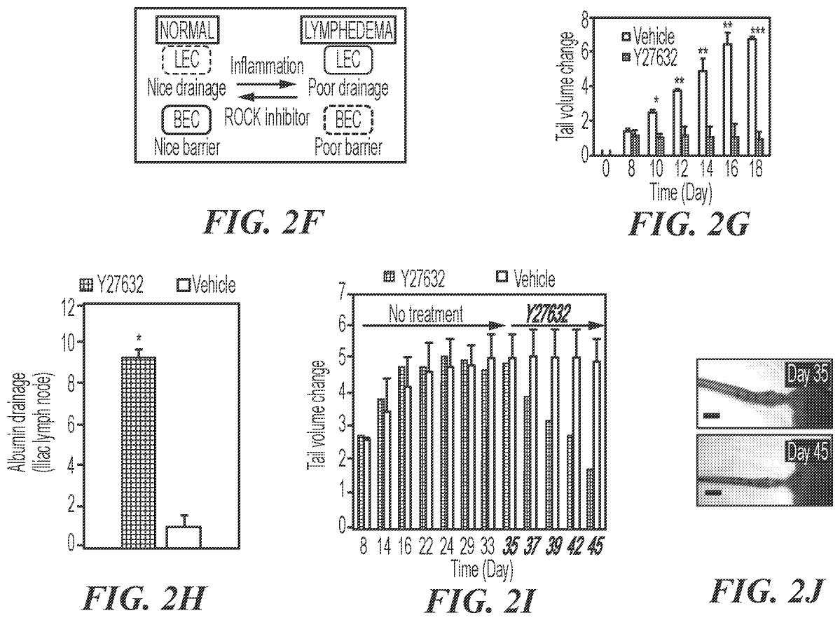

To overcome the issues outlined above, a microfluidic 3D culture model featuring an engineered lymphatic vessel that can drain interstitial fluid was developed. The device includes a polydimethylsiloxane (PDMS) housing and two parallel cylindrical channels within a 3D collagen hydrogel (FIG. 1A). Human dermal lymphatic endothelial cells (LECs) seeded in one channel generated an engineered lymphatic vessel (FIG. 1A) that expressed lymphatic endothelial markers with appropriate apical-basal orientation (FIG. 1A and FIG. 1B). Furthermore, stimulation with the lymphangiogenic vascular endothelial growth factor C (VEGFC) triggered lymphatic sprouting (FIG. 1C). To characterize the integrity of the lymphatic endothelium within the device, their junctional organization was examined by immunofluorescence. Staining for junctional adhesion molecule-A (JAM-A) and zonula occludens-1 (ZO-1), revealed weak tight junctions (TJs) in the LEC-generated lymphatic vessels, in contrast to strongly established TJs in the human dermal blood microvascular endothelial cell (BEC)-generated blood vessels as controls (FIG. 1D), similar to what has been describe of native lymphatics. Similarly, VE-cadherin staining to visualize adherens junctions (AJs) showed irregular undulating AJs in the lymphatic vessels versus smooth contiguous AJs in the blood vessels (FIG. 1E), as well as portal-like structure in only the lymphatics (FIG. 1E and FIG. 1F), reminiscent of the ‘button-like junctions’ in the initial lymphatic vessels observed in vivo (4, 5). Together, these data suggested the structural characteristics of a rudimentary lymphatic vessel.

To functionally assess this rudimentary lymphatic vessel required a measure of lymphatic drainage. To accomplish this, the second, cell-free channel was pressurized with a surrogate lymph fluid composed of fluorescently labeled lymph molecules, including fatty acids, phospholipids, albumin, and insoluble particles (data not shown). The interstitial fluid pressure triggered convective fluid transport into the engineered lymphatic vessel. Lymph fluid drained by the lymphatic vessel accumulated in the connected reservoirs and was collected for analysis. Human dermal blood microvascular endothelial cells-generated blood vessels or cell-free channels were used as controls. For all tested lymph molecules, engineered lymphatic vessels showed superior draining ability, compared to engineered blood vessels (FIG. 1G). Time-lapse microscopy of drainage further confirmed that fluid entered the luminal space of lymphatic vessels faster than that of blood vessels (FIG. 1H and data not shown). Surprisingly, whereas blood vessels appeared to be compressed by the increased interstitial fluid pressure, lymphatic vessel lumens widened under the same conditions (data not shown), consistent with in vivo observations that lymphatics dilate with enhanced interstitial fluid pressure (6)(7, 8). Together, these findings suggested that this lymphatics-on-chip recapitulated the structural and functional characteristics of native lymphatic vessels.

### Example 2: Identification and Use of Inhibitors of Inflammatory Cytokines In Vivo in Lymphedema

Inflammation is a primary driver of lymphedema (2, 9, 10), so the next set of experiments tested whether inflammatory cytokines could impair drainage in this system. Indeed, IL-2, GM-CSF, and G-CSF each markedly decreased lymphatic drainage (FIG. 1I), tightened AJs (FIG. 1J) and TJs (data not shown). To begin to characterize what signaling pathways might be involved in tightening the lymphatic junctions, inhibitors that might impact IL-2-induced junction assembly were identified (data not shown): PP2, a Src-family of protein tyrosine kinase inhibitor; Wortmannin, a PI3K inhibitor; Y27632, a ROCK inhibitor; GGTI298, a Rap1 inhibitor; NSC23766, a Rac1 inhibitor; Blebbistatin, a Myosin II inhibitor; and Cpd22, an integrin-linked kinase inhibitor. Only the ROCK inhibitor, Y27632, completely normalized the impaired drainage in IL-2 treated lymphatic vessels (data not shown). Y27632 also normalized GM-CSF and G-CSF treated conditions (FIG. 2A and FIG. 2B; and data not shown), and clinically used ROCK inhibitors Fasudil and Ripasudil (11), similarly improved lymphatic drainage (FIG. 2C), together suggesting a role for ROCK in inflammatory modulation of lymphatic junctions and drainage.

Interestingly, inflammatory cytokines are known to have the opposite effects on capillary blood vessels, increasing vessel permeability by loosening cell-cell junctions (12), which can also contribute to lymphedema (2). Indeed, in the engineered BEC-line vessels, IL-2, GM-CSF, and G-CSF all disrupted vascular barrier function (FIG. 2D and FIG. 2E). Here, treatment with Y27632 rescued barrier function (FIG. 2E and data not shown), suggesting that ROCK inhibition might ameliorate lymphedema, by simultaneously reducing fluid leakage from blood vessels and increasing lymphatic drainage (FIG. 2F).

To investigate the potential efficacy of ROCK inhibition in lymphedema, Y27632 was tested in an established mouse model of lymphedema (13, 14). Lymphedema was induced by surgical ligation of peripheral lymphatics in mouse tails (FIG. 2G). Treatment with Y27632 completely prevented lymphedema normally induced by the procedure (FIG. 2G), and further resulted in tenfold higher drainage to proximal lymph nodes as compared to untreated control mice (FIG. 2H). While these results suggested the potential to prevent lymphedema, whether ROCK inhibition could reverse existing lymphedema was also investigated. Tail-injured mice were left without any treatment for one month. Although a minority of animals recovered naturally, several animals established sustained tail swelling, exhibiting chronic lymphedema.

Treatment of these mice with Y27632 gradually improved and completely resolved the lymphedema by 2 weeks following treatment (FIG. 2I, FIG. 2J; and data not shown).

### Example 3: Characterization of ROCK2 and ROCK2 Inhibitor in LECs and BECs

While these results suggested a promising avenue for treating lymphedema, it remained unclear how ROCK activity could assemble lymphatic endothelial cell (LEC) junctions while having the opposite effect on blood endothelial cell (BEC) junctions. Without being bound by theory, it was postulated that these differences might arise from different functions of the two isoforms of ROCK, ROCK1 and ROCK2 (15). Under basal conditions, ROCK1 was detected at comparable levels in LECs and BECs, whereas ROCK2 was nearly undetectable in BECs (FIG. 3A). Interestingly, IL-2 in LECs substantially downregulated ROCK1, leaving ROCK2 as the dominant isoform (FIG. 3B). To test whether changes in ROCK1/2 levels might alter LEC junctions and drainage, the effects of siRNA knock-down of ROCK1/2 were examined (FIG. 3C). Indeed, knocking down ROCK1 impaired drainage in LECs, phenocopying the effects of IL-2 exposure, while loss of ROCK2 phenocopied Y27632, maintaining drainage regardless of IL-2 (FIG. 3D). These striking differences between ROCK1 and ROCK2 were also reflected by the phenotype of adherens junctions (FIG. 3E). Loss of ROCK1 tightened junctions while loss of ROCK2 loosened junctions. Given the prominent expression of ROCK1 in BECs, the effects of knocking down ROCK1 were examined also in these cells. As before, control BECs showed high barrier function (low permeability) in basal conditions that is lost with exposure to IL-2 (FIG. 3F). Consistent with the effects in LECs, knocking down ROCK1 allowed cells to maintain high barrier function (FIG. 3F) and intact junctions (FIG. 3G). Together, these data suggest that ROCK2 is required for cytokine-induced junction tightening in LECs, and inhibition of ROCK2 can normalize these junctions to support lymphatic drainage.

Next, by isolating endogenous ROCK2 via immunoprecipitation, an interaction with junctional adhesion molecule-A (JAM-A) and zonula occludens-1 (ZO-1) that emerges only in cytokine-exposed LECs was identified (FIG. 3H). ROCK1 was not in the junction complex, but knockdown of ROCK1 induced complex assembly phenocopying the effects of IL-2 (FIG. 3H). Immunofluorescence staining confirmed that IL-2 or ROCK1 knockdown induced ROCK2 localization with tight junctions (FIG. 3I). To test whether JAM-A was functionally important to the ROCK pathway, JAM-A was knocked down—LECs did not tighten junctions with IL-2 (FIG. 3J). These ROCK2/JAM-A/ZO-1 complexes were not observed in BECs (FIG. 3K). Finally, a ROCK2-specific small molecular inhibitor, KD025, in clinical trials phase II for treating chronic graft vs. host disease, idiopathic pulmonary fibrosis, and psoriasis, was treated in LECs under normal and IL-2 condition (FIG. 3L). KD025 (20 μM) reversed tightening of the LEC junctions (data not shown) and impaired drainage (FIG. 3L) against IL-2 challenge. Taken together, ROCK2 tightens lymphatic junctions when inflammatory signals are present by associating with JAM-A and ZO-1.

### Example 4: Characterization of Selective ROCK2 Inhibitor In Vivo

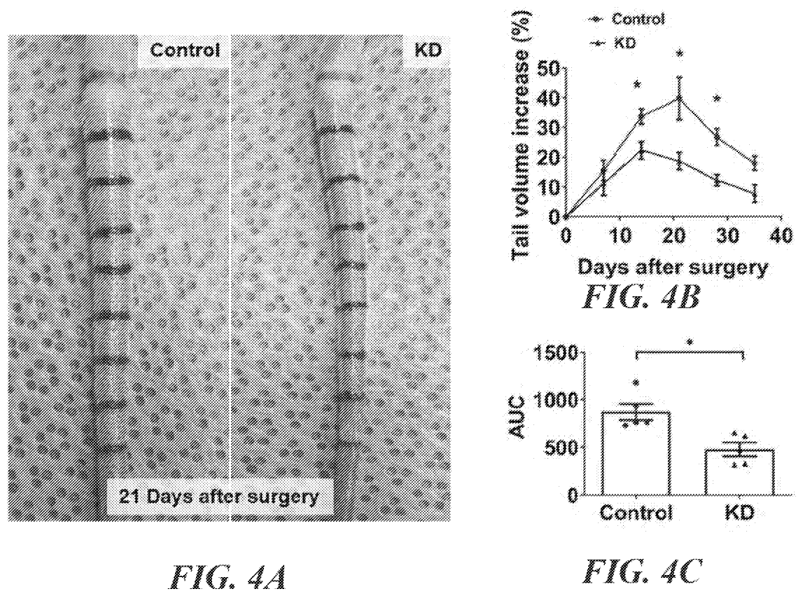

Administration of KD025 in mice reduced visible tail vein edema swelling following cautery injury (FIG. 4A). Tail volume changes were quantified (FIG. 4B), and drainage to the iliac lymph nodes was assesses 1 hour after intradermal injection of Alexa647-conjugated albumin into tails of mice treated with KD025 or vehicle (FIG. 4C).

Here, using a 3D organotypic model of lymphatic vessels that emulates lymphatic junctional structure and drainage function, the data shows that inflammatory cytokines trigger lymphatic endothelial cells to tighten their cell-cell junctions via a ROCK2/JAM-A/ZO-1 protein complex not found in blood endothelium. Given the limited clinical utility of ROCK inhibitors due to ROCK1-mediated relaxation of vascular smooth muscle leading to hypotension (11), the findings here suggest that ROCK2-specific therapeutics, such as KD025, could be used to treat lymphedema with less toxicity. This study highlights the central role of cell-cell junctions in regulating the passage of fluid from blood vessels through interstitial spaces and back into the lymphatic circulation and identifying the special mechanisms that cells use to dynamically regulate these adhesions will be central to normalizing perfusion. Furthermore, the work underscores how recapitulating appropriate architectures can enable the emergence of multicellular functions previously only observed in vivo, and the advancement of such biomimetic cultures is proving to play a key role in bridging traditional in vitro and in vivo models of physiology and disease.

### REFERENCES

1. T. Tammela, K. Alitalo, Lymphangiogenesis: Molecular mechanisms and future promise. Cell 140, 460-476 (2010).
2. S. H. Ridner, Pathophysiology of lymphedema. Semin Oncol Nurs 29, 4-11 (2013).
3. S. G. Rockson, Update on the biology and treatment of lymphedema. Curr Treat Options Cardiovasc Med 14, 184-192 (2012).
4. P. Baluk et al., Functionally specialized junctions between endothelial cells of lymphatic vessels. J Exp Med 204, 2349-2362 (2007).
5. H. Pflicke, M. Sixt, Preformed portals facilitate dendritic cell entry into afferent lymphatic vessels. J Exp Med 206, 2925-2935 (2009).
6. L. V. Leak, Electron microscopic observations on lymphatic capillaries and the structural components of the connective tissue-lymph interface. Microvasc Res 2, 361-391 (1970).
7. M. Skobe, M. Detmar, Structure, function, and molecular control of the skin lymphatic system. J Investig Dermatol Symp Proc 5, 14-19 (2000).
8. J. W. Breslin, Mechanical forces and lymphatic transport. Microvasc Res 96, 46-54 (2014).
9. S. G. Rockson, The lymphatics and the inflammatory response: lessons learned from human lymphedema. Lymphat Res Biol 11, 117-120 (2013).
10. C. L. Ly, R. P. Kataru, B. J. Mehrara, Inflammatory Manifestations of Lymphedema. Int J Mol Sci 18, (2017).
11. Y. Feng, P. V. LoGrasso, O. Defert, R. Li, Rho Kinase (ROCK) Inhibitors and Their Therapeutic Potential. J Med Chem 59, 2269-2300 (2016).
12. Y. Berthiaume et al., Pulmonary edema during IL-2 therapy: combined effect of increased permeability and hydrostatic pressure. Am J Respir Crit Care Med 152, 329-335 (1995).
13. M. Schneider, A. Ny, C. Ruiz de Almodovar, P. Carmeliet, A new mouse model to study acquired lymphedema. PLoS Med 3, e264 (2006).
14. W. Tian et al., Leukotriene B4 antagonism ameliorates experimental lymphedema. Sci Transl Med 9, (2017).
15. O. Nakagawa et al., ROCK-I and ROCK-II, two isoforms of Rho-associated coiled-coil forming protein serine/threonine kinase in mice. FEBS Lett 392, 189-193 (1996).
16. J. Zhang et al., Role of connexin 43 in vascular hyperpermeability and relationship to Rock1-MLC20 pathway in septic rats. Am J Physiol Lung Cell Mol Physiol 309, L1323-1332 (2015).
17. H. Peng et al., Simvastatin alleviates hyperpermeability of glomerular endothelial cells in early-stage diabetic nephropathy by inhibition of RhoA/ROCK1. PLoS One 8, e80009 (2013).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A method comprising administering a selective Rho-associated kinase 2 (ROCK2) inhibitor to a subject having or at risk of a condition associated with impaired lymphatic drainage.

2. The method of claim 1, wherein the selective ROCK2 inhibitor is administered in an amount effective for alleviating a symptom associated with impaired lymphatic drainage.

3. The method of claim 2, wherein the symptom is swelling in a body part of the subject.

4. A method comprising administering a selective ROCK2 inhibitor to a subject in an amount effective to rescue vascular barrier function.

5. The method of claim 4, wherein the amount of the selective ROCK2 inhibitor is effective in reducing fluid leakage from blood vessels in the subject, relative to a control.

6. The method of claim 4 or 5, wherein the amount of the selective ROCK2 inhibitor is effective in increasing lymphatic drainage in the subject, relative to a control.

7. A method comprising administering a selective ROCK2 inhibitor to a subject in an amount effective for improving lymphatic drainage at a lymphatic junction in the subject, relative to a control.

8. A method comprising administering a selective ROCK2 inhibitor to a subject in an amount effective in an amount effective for preventing formation of tight lymphatic junctions in the subject.

9. The method of any one of the preceding claims, wherein the subject has a condition associated with impaired lymphatic drainage.

10. The method of claim 9, wherein the condition is edema.

11. The method of claim 9, wherein the condition is lymphedema.

12. The method of claim 11, wherein the lymphedema is primary lymphedema.

13. The method of claim 11, wherein the lymphedema is secondary lymphedema.

14. The method of any one of the preceding claims, wherein the selective ROCK2 inhibitor is administered orally.

15. The method of claim 14, wherein the selective ROCK2 inhibitor is formulated as a tablet.

16. The method of any one of the preceding claims, wherein the selective ROCK2 inhibitor is administered as a dose of 200-400 mg.

17. A method comprising contacting dermal lymphatic endothelial cells with a selective Rho-associated kinase 2 (ROCK2) inhibitor in an amount effective for preventing formation of tight lymphatic junctions.

18. A method comprising contacting a tight lymphatic junction with a selective Rho-associated kinase 2 (ROCK2) inhibitor in an amount effective for improving lymphatic drainage at the lymphatic junction, relative to a control.

19. The method of any one of the preceding claims, wherein the selective ROCK2 inhibitor binds to ROCK2 and inhibits ROCK2 serine/threonine kinase activity.

20. The method of any one of the preceding claims, wherein the selective ROCK2 inhibitor is selected from polypeptide inhibitors, polynucleotide inhibitors, and small molecule inhibitors.

21. The method of claim 20, wherein the selective ROCK2 inhibitor is a small molecule inhibitor.

22. The method of claim 21, wherein the small molecule inhibitor binds ROCK2 with an IC₅₀ value of at 50 nM-150 nM.

23. The method of claim 21 or 22, wherein the small molecule inhibitor binds ROCK2 but does not bind protein kinase A (PKA), protein kinase G (PKG), protein kinase C (PKC), or myotonic dystrophy kinase-related CDC42-binding kinase (MRCK).

24. The method of any one of claims 21-23, wherein the small molecule inhibitor binds ROCK1 with an IC₅₀ value of 20,000 nM-25,000 nM.

25. The method of any one of claims 21-24, wherein the small molecule inhibitor is 2-[3-[4-(1H-indazol-5-ylamino)-2-quinazolinyl]phenoxy]-N-(1-methylethyl)-acetamide (KD025).

26. The method of any one of claims 21-24, wherein the small molecule inhibitor is selected from Compounds 1-559, or a pharmaceutically acceptable salt, stereoisomer, tautomer, isotopically labeled derivative, solvate, hydrate, polymorph, co-crystal, or prodrug thereof.

27. The method of claim 26, wherein the small molecule inhibitor is selected from Compounds 1-559, or a pharmaceutically acceptable salt thereof.
